(19) United States
(12) Patent Application Publication
(10) Pub. No.: US 2026/0308621 A1
(43) Pub. Date: Oct. 8, 2026

(54) COMPOSITIONS COMPRISING SMYD1 AND METHODS OF USE THEREOF

(71) Applicant: UNIVERSITY OF UTAH RESEARCH FOUNDATION, Salt Lake City, UT (US)

(72) Inventors: Sarah FRANKLIN, Salt Lake City, UT (US); Marta W. SZULIK, Salt Lake City, UT (US)

(21) Appl. No.: 19/481,373

(22) PCT Filed: May 3, 2024
(86) PCT No.: PCT/US2024/027886

§ 371 Date: Nov. 4, 2025

Related U.S. Application Data
(60) Provisional application No. 63/500,206, filed on May 4, 2023.

(51) Int. Cl. C12N 15/86; A61K 35/76; A61K 38/45; A61K 48/00; A61P 9/10

(52) U.S. Cl. CPC C12N 15/86; A61K 35/76; A61K 38/45; A61K 48/0058; A61P 9/10; C12N 2750/14143; C12N 2830/008; C12N 2830/48; C12N 2830/50; C12Y 201/01

(57) ABSTRACT

Disclosed are methods of reducing infarct size in a subject comprising increasing SMYD1 in the subject. Disclosed are methods of reducing myocyte death in a subject comprising increasing SMYD1 in the subject. Disclosed are method of enhancing mitochondrial respiration in a cell comprising increasing SMYD1 in the cell. Disclosed are methods of increasing ATP production in a cell comprising increasing SMYD1 in the cell. Disclosed are methods of protecting a heart from ischemic injury comprising increasing SMYD1 in the heart. Disclosed are vectors comprising a nucleic acid construct, wherein the nucleic acid construct comprises a nucleic acid sequence encoding SMYD1.

### CROSS-REFERENCE TO RELATED APPLICATIONS

This application claims the benefit of U.S. Provisional Patent Application No. 63/500,206, filed May 4, 2023, each of which is incorporated by reference herein in its entirety.

### STATEMENT REGARDING FEDERALLY SPONSORED RESEARCH

This invention was made with government support under R01 HL161045 and R01 HL130424 awarded by the National Institutes of Health. The government has certain rights in the invention.

### BACKGROUND

Ischemic heart disease is the primary cause of chronic heart failure (HF), one of the most devastating conditions with mortality exceeding 50% in patients diagnosed with advanced HF. Although significant advancements have been made clinically to reduce myocardial injury from ischemia and subsequent reperfusion (including stents and bypass surgery), currently 25% of patients will die or develop heart failure within 1 year. Therefore, understanding the molecular pathways that contribute to or protect from ischemic injury may be useful for developing novel therapies to either prevent, further slow the progression, or reverse the functional deficiencies of the failing heart.

There have been many studies addressing molecular mechanisms underlying ischemic injury that focus on myocardial metabolism and energetics, however very little is known about the epigenetic regulators, which induce transcriptional changes in the myocyte genome and drive these metabolic changes. While advancements have been made in the field of epigenetics in general, the proteins responsible for regulating chromatin and their subsequent effects on cellular and organ remodeling during ischemic heart disease are largely unknown. Studies of acute myocardial infarction in animal models have shown that targeting key pathways in the cardiomyocyte can diminish ischemic injury. Specifically, early reports utilizing histone deacetylase inhibitors revealed that heart disease progression could be attenuated upon treatment in animal models, however the ubiquitous expression of these proteins and the non-specific nature of the inhibitors has made them unfeasible as a therapeutic tool in the heart, thus far. In contrast, the lysine methyltransferase SMYD1 is a myocyte-specific epigenetic regulator, which has been shown to regulate gene expression in the cardiomyocyte. Originally, SMYD1 was reported to play a role in embryonic cardiac development, however, more recently SMYD1 has been shown to be differentially expressed in human heart failure patients and in mouse models of heart disease. Previous work focused on the characterization of Smyd1-knockout mice and demonstrated that cardiomyocyte-specific loss of Smyd1 in the adult murine heart leads to myocyte growth and heart failure, which is preceded by dysregulation of cardiac metabolism and a reduction in mitochondrial respiration capacity. In addition, cultured cells showed that SMYD1's ability to regulate mitochondrial respiration was due, in part, to its transcriptional control of Ppargc1α (a regulator of mitochondrial number and energetics). However, the physiological ramifications of SMYD1 gain-of-function in an animal model as well as its role in response to ischemic injury has never been elucidated.

### BRIEF SUMMARY

Disclosed are methods of reducing infarct size in a subject comprising increasing SMYD1 in the subject.

Disclosed are methods of reducing myocyte death in a subject comprising increasing SMYD1 in the subject.

Disclosed are method of enhancing mitochondrial respiration in a cell comprising increasing SMYD1 in the cell

Disclosed are methods of increasing ATP production in a cell comprising increasing SMYD1 in the cell.

Disclosed are methods of protecting a heart from ischemic injury comprising increasing SMYD1 in the heart.

Disclosed are vectors comprising a nucleic acid construct, wherein the nucleic acid construct comprises a nucleic acid sequence encoding SMYD1.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several aspects of the disclosed method and compositions and together with the description, serve to explain the principles of the disclosed method and compositions.

FIGS. 1A-1G show SMYD1 is downregulated in failing heart. FIG. 1A) Transcript variants of SMYD1 in mouse and human hearts. Two splice variants of Smyd1 in mouse heart differ by 13 amino acids insert in Smyd1a (orange bar), which is conserved in the human transcript. Location of primers specific to either Smyd1 isoform is indicated by underlined F (red and blue, below mouse Smyd1a and Smyd1b sequences, respectively) and R (purple). FIG. 1B-D) Evaluation of SMYD1 expression in human hearts that underwent LVAD treatment by (B) qRT-PCR and (FIG. 1C. D) western blot (WB). Non-resp. indicates non-responders and Resp. indicates responders. Asterisk * indicates p<0.05, n=5/Control, n=10/Failure (qRT-PCR), and n=5/group (WB). FIG. 1E-G) qRT-PCR (FIG. 1E) and western blot evaluation (FIG. 1F) with quantification (FIG. 1G) of SMYD1 levels in mouse hearts 5 weeks after permanent occlusion (PO) of the LAD or Sham surgery. Asterisk * indicates p<0.05, n=6-7.

FIGS. 2A-2I show generation of inducible, cardiomyocyte-specific SMYD1a overexpression in mice. FIG. 2A shows constructs for human SMYD1, mouse SMYD1a and mouse SMYD1b. Transgenic mice were generated by crossing animals expressing a reverse tetracycline-controlled transactivator (rtTA) under the aMHC promoter with mice carrying the Smyd1a-FLAG gene under the modified aMHC promoter containing a TRE element. These mice express Smyd1a-FLAG only upon doxycycline (FIG. 2D) administration (80 mg/kg/day). FIG. 2B) Genotyping of mice carrying the Smyd1a-FLAG and rtTA alleles. Doxycycline (DOX) induced expression of mouse SMYD1 isoforms was confirmed by RT-qPCR in FIG. 2C) cardiac and FIG. 2D) skeletal muscle showing overexpression specific to mouse heart. Asterisk * indicates p<0.05, n=3-4. Western blotting (FIG. 2E) and quantification (FIG. 2F) of SMYD1 in mouse cardiac tissue 3 weeks after doxycycline treatment. Asterisk * indicates p<0.05, n=3. Long term doxycycline treatment maintains SMYD1 expression at ~2-fold increase in transgenic (TG) mice as compared to wild type (WT) mice, as shown by FIG. 2G) representative western blotting (quantified in FIG. 2H), and FIG. 2I) LC/MS-MS, n=3-4.

FIGS. 3A-3S shows SMYD1a overexpression protects heart from ischemic injury. FIG. 3A) Mice were fed doxycycline chow for 2 weeks, subjected to permanent occlusion (PO) of the LAD, monitored weekly by echocardiography and hearts were harvested at 5 weeks after PO surgery. Biological replicates for phenotyping studies are presented in the table. FIG. 3B) Heart weight:body weight and FIG. 3C) heart weight:tibia length ratios show no significant changes between experimental groups. FIG. 3D) Cardiac function was measured by echocardiography and represented by FIG. 3D) echocardiographs and measured FIG. 3E) at the view of middle point of short or long axis from M-mode to determine FIG. 3F) ejection fraction and G) fractional shortening. Echo data show preserved cardiac function in transgenic (TG) mice when compared to littermate controls (WT) that underwent PO surgery. Asterisk * indicates p<0.05. H) Five serial sections of the hearts were acquired at 5 weeks from the base (FIG. 3I) to apex (V) and these representative images show that SMYD1a mitigates ischemic injury in TG mice compared to WT control which lack the rtTA transgene and are also maintained on DOX chow. Bar=100 mm. FIG. 3I) The ischemic injury from this surgical model was quantified by categorizing all IV sections into severe, medium, and mild infarct categories and representative images further confirm a reduction in infarct size in TG mice. Bar=100 mm. FIG. 3J) Infarct size was quantified in Masson's trichrome-stained sections by measuring the midline length of injury as a percentage of total midline left ventricle circumference. Asterisk * indicates p<0.05, n=5-9. FIG. 3K) The presence and extend of infarct lesions was scored using H&E-stained slides; scores were summed for each animal, n=3-4. FIG. 3L) Apoptosis in cardiac tissue harvested 48 h after PO of the LAD was detected by TUNEL assay and TUNEL positive cells (green, pointed with arrows) were quantified (FIG. 3M) in all experimental groups indicating significant decrease in apoptosis in TG PO mice. Asterisk * indicates p<0.05, n=9-15. FIG. 3N) Fetal genes: Nppa, Myh6, Myh7 and Atp2a2, and FIG. 3O) fibrotic genes: Vim and Colla1 were measured by qRT-PCR and values expressed as relative mRNA intensities relative to WT control mice. Asterisk * indicates p<0.05, n=11-19. FIG. 3P, Q) Permanent occlusion had no effect on the SMYD1 expression levels in TG mice. N=3-4. FIG. 3R,S) Neither overexpression of SMYD1a nor permanent occlusion effected global levels of histone H3K4 trimethylation as shown by FIG. 3R) western blotting and FIG. 3S) quantification. Asterisk * indicates p<0.05, n=3-4.

FIGS. 4A-4M show SMYD1a overexpression enhances mitochondrial respiration and lowers ROS production. FIG. 4A) Model of the mitochondrial electron transport chain (ETC) and ATP synthesis. IMS—intermembrane space, IMM—inner mitochondrial membrane, MM—mitochondrial matrix. Western blotting showing expression levels of SMYD1a in H9c2 cardiomyoblasts that were transduced with Ad-SMYD1a-FLAG. C-F) Cell Mito Stress Test was conducted using a Seahorse Bioscience XFe96 analyzer by sequentially injecting 1 mM oligomycin, 5 mM FCCP, and 1 mM rotenone+antymycin A inhibitors. H9c2 cardiomyoblasts were transduced with Ad-SMYD1a and oxygen consumption rates (OCR) were recorded and quantified in the presence of FIG. 4C, D) pyruvate or FIG. 4E,F) palmitate as substrates. Quantitative analysis (FIG. 4D, F) of mitochondrial OCR from H9c2 cells overexpressing SMYD1a indicates significant increases in respiration in response to various inhibitors for both substrates. Asterisk * indicates p<0.05, t indicates p=0.08, n=5. FIG. 4G-J) Representative respiration traces in the Complex I/II-linked OXPHOS state (FIG. 4G) and fatty acid oxidation (FAO)-linked respiration (FIG. 4I) detected by Oroboros O2k oxygraph and quantitative analysis (FIG. 4H, J) of mitochondrial OCR from TG and WT control groups showing increased oxygen consumption in TG mice as compared to WT controls. Malate-pyruvate (MP), adenosine diphosphate (ADP), glutamate (G), succinate (S), carbonyl cyanide m-chlorophenyl hydrazone (FCCP), malate, L-carnitine, palmitoyl-L-camitine (FAT) were used as substrates. FIG. 4K) Evaluation of ATP production rates in isolated mitochondria from TG and WT mice subjected to PO show that molar amounts of ATP produced per mole of atomic oxygen consumed (known as ATP:O ratio) is increased in TG mice 24 h after PO. FIG. 4L, M) ROS levels are lower in H9c2 cardiomyoblasts that overexpress SMYD1a, and in hypoxic conditions, as measured by relative MitoSOX fluorescence and quantified by normalization to control. Asterisk **** indicates p<0.0001, n=353-622.

FIGS. 5A-5I show SMYD1a overexpression prevents a decline in metabolic enzymes downstream of PGC-1a. FIG. 5A) Expression of PGC-1a was quantified via FIG. 5A) qPCR and FIG. 5B,C) western blotting in cardiac tissue from TG and WT mice 48 h after permanent occlusion of the LAD or Sham surgeries. These data show that PGC-1α expression is significantly decreased in response to ischemic stress (PO); however, overexpression of SMYD1a is capable of maintaining PGC-1α at basal levels. Asterisk * indicates p<0.05, n=3-6. FIG. 5D) Publicly available ChIP-Seq data for histone H3K4me3 at the Ppargc1α promoter was used to design primers to use in ChIP-qPCR for SMYD1a-FLAG, H3K4me3 and H3K9me3 in cardiac tissue from TG and WT mice showing E) SMYD1a binding at the Ppargc1α promoter which FIG. 5F) increases trimethylation of histone H3K4 under basal conditions. FIG. 5G) H3K9me3 ChIP was used as a negative control. Asterisk * indicates p<0.05, n=4-6. FIG. 5H) Overexpression of SMYD1a rescues expression of genes involved in OXPHOS and fatty acid oxidation which are regulated by ERRα or PPARα transcription factors, respectively, and downregulated in response to ischemic injury. Asterisk * indicates p<0.05, † indicates p<0.08, n=4-6. FIG. 5I). Under basal conditions. SMYD1a, through its histone methyltransferase activity, regulates expression of PGC-1α and its downstream targets which results in maintained metabolic homeostasis and cardiac function.

FIGS. 6A-6O show SMYD1a overexpression regulates mitochondrial respiration by increased formation of supercomplexes and cristae. FIG. 6A) Diagram showing factors influencing mitochondrial respiration. FIG. 6B) Western blotting evaluation of the electron transport chain (ETC) complex subunits using an antibody cocktail showing no significant changes in abundance of ETC complexes in TG mice or after PO, n=3. FIG. 6C) Immunoblotting for additional ETC subunits showed FIG. 6D) modest changes in a few subunits (NDUFS2, NDUFV1), but no change in most of those examined, n=3. E-F) Mitochondrial biogenesis is absent in transgenic SMYD1a mice as confirmed by three markers: FIG. 6E) mitochondrial DNA quantified by ND1 and 16SrRNA via qPCR, n=6, and FIG. 6F) citrate synthase activity in TG and WT mice which showed no change after 2 weeks of DOX induced SMYD1a overexpression, n=3-4. FIG. 6G-I) SMYD1a overexpression increases supercomplex formation as determined by blue-native PAGE gel stained with FIG. 6G). Coomassie, evaluated for FIG. 6H) Complex I activity and FIG. 6I) quantified in WT and TG mice 48 hours after permanent occlusion of the LAD or Sham surgery. Asterisk * indicates p<0.05, n=3-4. FIG. 6J) Electron micrographs of cardiac tissue from WT, SMYD1a transgenic mice and Smyd1 KO mice show that while loss of SMYD1 leads to loss of cristae structure, SMYD1a overexpression leads to FIG. 6K) larger mitochondria with FIG. 6L) more dense, narrower cristae. Asterisk * indicates p-value p<0.05, n=3-7. FIG. 6M,N) Western blotting and quantification analysis showing increased expression of OPA1 and MFN2 and no change in mitofillin expression in TG mice, as compared to WT controls. Asterisk * indicates p<0.05, n=3. FIG. 6O) SMYD1a overexpression leads to formation of ETC supercomplexes and more dense and narrower cristae that results in increased mitochondrial respiration and ATP production through which it ultimately reduces ischemic injury and pathological remodeling.

FIGS. 7A-7N shows SMYD1a regulates OPA1 expression in the cardiomyocyte. FIG. 7A) OPA1 oligomers bind the inner mitochondrial membrane (IMM) at cristae junctions and regulate cristae structure. Overexpression of OPA1 has been shown to alter cristae morphology leading to reduced cristae width, increased cristae number and enhanced respiration by stabilizing supercomplex formation (right). Conversely, loss of OPA1 leads to reduced respiration and ATP, loss of cristae structure and cell death (left). (FIG. 7B-D) Western blotting analysis showing changes in OPA1 expression which is FIG. 7B,C) increased in SMYD1a TG mice and FIG. 7B,D) decreased in Smyd1 knockout (KO) mice at the (FIG. 7D) protein and (FIG. 7E) transcript level. Asterisk * indicates p<0.05, n=2-3. (FIG. 7F-I) Cultured H9c2 cardiomyoblasts were transfected with FIG. 7F) either scr-siRNA (control) or Smyd1-siRNA for 72 h or FIG. 7G) transduced with either empty virus (Control) or SMYD1a-FLAG adenovirus (Ad-SMYD1a) for 72 h. Both FIG. 7H) wester blotting and FIG. 7I) qPCR analysis show that siRNA-mediated Smyd1 knockdown in H9c2 cells led to significant downregulation of OPA1 (FIG. 7F, H, I) whereas overexpression of SMYD1a increased OPA1 expression (G,H,I). Asterisk * indicates p<0.05, n=3. FIG. 7J) Previous ChIP-Seq studies provided enrichment data for histone H3K4me3 at the Opal promoter which was used to design primers for chromatin immunoprecipitation (ChIP) and qPCR. ChIP-qPCR for adenovirus-mediated SMYD1a overexpression (using FLAG antibody) show FIG. 7K) enrichment in the promoter region of Opal; however, FIG. 7L) no corresponding enrichment of histone H3 lysine K4 trimethylation was detected in these regions. M) Histone H3K9me3 and FIG. 7N) random intergenic and control TBP promoter were used as negative controls to show that SMYD1 is not enriched by ChIP-qPCR at either of these regions. Asterisk * indicates p<0.05, n=4-6.) Overexpression of SMYD1a partially rescues the downregulation of cellular respiration caused by silencing of Opal, as shown by the Cell Stress Test in H9c2 cardiomyoblasts. Asterisk * indicates p<0.05, n=4-6.

FIG. 8 shows a schematic of SMYD1a's role in the heart. Top panel: Under basal conditions SMYD1a maintains metabolic homeostasis by regulating the expression of Ppargc1α through methylation of histone H3K4 at the promoter and its downstream targets including electron transport chain (ETC) subunits. Bottom panel: Overexpression of SMYD1a regulates OPA1 expression which mediates cristae remodeling and supercomplex formation of respiratory chain complexes. This leads to enhanced mitochondrial respiration and ATP production to protect from ischemic injury and pathological remodeling in the heart.

FIGS. 9A-9C show SMYD1a overexpression attenuates apoptotic cell death. Apoptosis was detected by TUNEL assay in (FIG. 9A) cardiac tissue harvested 48 h after PO (or Sham) surgery and in (FIG. 9B) H9c2 cardiomyoblasts that were overexpressing SMYD1a (by adenovirus) and subjected to hypoxic conditions. The results indicated significant decrease in apoptosis in TG PO mice when compared to WT PO group or in H9c2 cells overexpressing SMYD1a when compared to controls. DAPI staining shown in blue, cardiac troponin staining shown in red, TUNEL staining shown in GREEN(and FIG. 9C) and arrows point to TUNEL positive cells. Neg.Ctrl. indicates negative control and Pos.Ctrl. indicates positive control. Asterisk * indicates p<0.05, n=9-15.

FIG. 10A-10B show SMYD1a overexpression has no effect on angiogenesis in TG mice. FIG. 10A, B) Markers of angiogenesis FIG. TOA) Vegfa and FIG. 10B) Fgf-2 were measured by qRT-PCR and values expressed as relative mRNA intensities relative to WT control mice. n=4-6.

FIGS. 11A-11B show SMYD1a overexpression has no effect on expression of SMYD family members. FIG. 11A) SMYD family members: Smyd2, Smyd3, Smyd4 and Smyd5 were measured by qRT-PCR and values expressed as relative mRNA intensities relative to WT control mice. Asterisk * indicates p<0.05 to sham control, n=4-6. FIG. 11B) As indicated by western blotting, overexpression of SMYD1a or permanent occlusion (PO) had no effect on levels of SMYD2, SMYD4 and p53, however there is a significant increase in HSP90 in TG Sham group as compared to WT controls. Asterisk * indicates p<0.05, n=3-4.

FIGS. 12A-12G show basal expression of SMYD1a in cardiac tissue and H9c2 cardiomyoblasts. Expression at both the transcript (FIG. 12A) and protein (FIG. 12B, C) levels shows that SMYD1's basal expression is much higher in cardiac tissue as compared to H9c2 cardiomyoblasts. FIG. 12D, E) Adenovirus mediated increasing overexpression of SMYD1a in H9c2 cells detected by western blotting and quantified. FIG. 12F) Cell Mito Stress test was conducted using a Seahorse Bioscience XFe96 analyzed by sequentially injecting 1 mM oligomycin, 5 mM FCCP, and 1 mM rotenone+antymycin A inhibitors. H9c2 cardiomyoblasts were transduced with Ad-SMYD1a and oxygen consumption rates (OCR) were recorded and quantified in the presence of pyruvate as substrate. FIG. 12G) Quantitative analysis of mitochondrial OCR from H9c2 cells overexpressing SMYD1a indicates significant increases in respiration starting at MOI=50. Asterisk * indicates p<0.05, n=5-12.

FIGS. 13A-13C show SMYD1a binds to the promoter of Ppargc1α and regulates its expression through histone H3K4me3 in isolated cells. FIG. 13A) Adenoviral transduction of NRVMs led to a robust SMYD1a expression, as confirmed by western blotting. FIG. 13B) Publicly available ChIP-Seq data for histone H3K4me3 at the Ppargc1α promoter were used to design primers to use in ChIP-qPCR for SMYD1a-FLAG and histone H3K4me3 in isolated NRVMs showing FIG. 13C) binding at the Ppargc1α promoter, which increases trimethylation of histone H3K4me3 under basal conditions. Asterisk * indicates p<0.05. n=2.

FIGS. 14A-14B show SMYD1a overexpression increases supercomplex formation in H9c2 cardiomyoblasts but knockdown of Smyd1 has no effect. Blue-native PAGE gel stained with Coomassie, evaluated for Complex I activity and quantified in H9c2 cardiomyoblasts 48 h after FIG. 14A) adenovirus mediated overexpression of SMYD1a or FIG. 14B) si-RNA driven knockdo

FIGS. 15A-15B show cardiac specific deletion of Smyd1. Mice fed tamoxifen diet for 3 weeks show nearly a complete loss of SMYD1 protein in the heart, as demonstrated by western blotting. WT—wild type, KO—Smyd1 knockout. Asterisk * indicates p<0.05, n=3.wn of Smyd1. Asterisk * indicates p<0.05, n=3.

FIGS. 16A-16D show SMYD1a overexpression induces mitochondrial calcium uptake but remains unchanged in hypoxic conditions. FIG. 16A) Representative images showing control and SMYD1a overexpressing H9c2 cardiomyoblasts stained with the mitochondrial Ca2+ sensor, XRhod1. FIG. 16B) Changes is mitochondrial Ca2+ levels following XRhod1 staining in control and SMYD1a overexpressing H9c2 cardiomyoblasts. Asterisk * indicates p<0.05, **** indicates p<0.0001, n=408-485. FIG. 16C) A representative calcium retention trace of control and SMYD1a overexpressing H9c2 cardiomyoblasts. Black arrows indicate 5 mM Ca2+ injections. Mitochondrial viability was measured as ΔΨ/TMRM fluorescence. The graph is representative of five experiments each with 5E-5 cells. FIG. 16D) Comparison of calcium retention before the triggering of an MPT event in control and SMYD1a overexpressing cells. Asterisk ** indicates p<0.01, n=3.

FIGS. 17A-17D show schematic diagram representing possible mechanisms by which SMYD1a could regulate gene expression. FIG. 17A) Canonically, SMYD1a activates transcription by trimethylation of histone H3 on lysine K4. FIG. 17B) However, based od data from other methyltransferases, it is possible that SMYD1a targets another histone PTM through which it regulates gene expression. FIG. 17C) In addition, SMYD1a could also regulate transcription by methylating other chromatin binding protein or FIG. 17D) through methyltransferase-independent mechanism, as a component of a chromatin binding complex. All of these four mechanisms have previously been shown to be utilized by methyltransferases to regulate gene expression.

FIGS. 18A-18B show knockdown of Opal. si-RNA-mediated knockdown of Opal in H9c2 cardiomyoblasts showed significantly reduced levels of mRNA expression evaluated by (FIG. 18A) qRT-PCR and OPA1 protein as determined by (FIG. 18B) western blot analysis. Asterisk * indicates p<0.05, n=3.

FIG. 19 shows example designs/constructs for the Smyd1 gene therapy using AAV9-based delivery platforms.

FIG. 20 shows example designs/constructs for the Smyd1 gene therapy using viral-based delivery platforms. Examples of capsids include: AAV1-9, AAVrh10, AAVrg74, and derivatives thereof, used alone or in combination.

### DETAILED DESCRIPTION

The disclosed method and compositions may be understood more readily by reference to the following detailed description of particular aspects and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular aspects only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid sequences, nucleic acid constructs, or vectors is disclosed and discussed and a number of modifications that can be made to a number of molecules including the amino acids are discussed, each and every combination and permutation of the nucleic acid sequences, nucleic acid constructs, or vectors and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E. A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of aspects of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular aspects only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a vector” includes a plurality of such vectors, reference to “the vector” is a reference to one or more vectors and equivalents thereof known to those skilled in the art, and so forth.

The word “or” as used herein means any one member of a particular list and also includes any combination of members of that list.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

By “treat” is meant to administer a vector, composition, or nucleic acid of the invention to a subject, such as a human or other mammal (for example, an animal model), that has an increased susceptibility for developing a disease, disorder or infection in order to prevent or delay onset of the disease disorder or infection, prevent or delay a worsening of the effects of the disease, disorder or infection, or to partially or fully reverse the effects of the disease, disorder or infection. In some aspects, treat can mean to ameliorate a symptom of a disease, disorder or infection.

By “prevent” is meant to minimize the chance that a subject who has an increased susceptibility for developing a disease, disorder or infection will actually develop the disease, disorder or infection.

As used herein, the term “subject” or “patient” can be used interchangeably and refer to any organism to which a virus or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles: zoo and wild animals). Typically, “subjects” are animals, including mammals such as humans and primates, and the like.

As used herein, the terms “administering” and “administration” refer to any method of providing a disclosed composition of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed vector so as to treat a subject.

In some aspects, “gene therapy” can be understood to mean a treatment of a subject's body or isolated elements of a subject's body, for example isolated tissues/cells, by nucleic acids encoding a peptide or protein. It typically may comprise at least one of the steps of a) administration of a nucleic acid directly to the subject—by whatever administration route—or in vitro to isolated cells/tissues of the subject, which results in transfection/transduction of the subject's cells either in vivo, ex vivo, or in vitro; b) transcription and/or translation of the introduced nucleic acid molecule; and optionally c) re-administration of isolated, transfected cells to the subject, if the nucleic acid has not been administered directly to the subject. The term “gene therapy” as used herein typically comprises treatment as well as prevention or prophylaxis of a disease.

SMYD1 refers to any species of SMYD1. Table 4 provides the correlation between human SMYD1 across species. As used throughout, SMYD1 can refer to any SMYD1 protein or gene of any species or a variant thereof. In some aspects, variant SMYD1 retains the functional characteristics of wild type SMYD1. Thus, everywhere SMYD1 is referred to throughout, any SMYD1 species can be intended. In some aspects. SMYD1 can refer to any isoform of SMYD1, for example isoform 1 or isoform 2 of human SMYD1.

TABLE 4
Characteristics of SMYD1 isoforms across different species.

| | RefSeq | | RefSeq | | | | |
|---|---|---|---|---|---|---|---|
| | Official | RefSeq | Transcript | Common | | | |
| | Symbol and | Transcript | Accession | Protein | | | Tissue/ |
| Species | Gene ID | Variant | Number | Name | Synonyms | Role | Cell Type |
| Homo | SMYD1 | Isoform 1 | NM_198274.4 | Smyd1 | Smyd1 | Cardiac growth and | Fetal |
| sapiens | ID: 150572 | Isoform 2 | NM_001330364.2 | | | development (38, 39) | Heart |
| (Human) | | | | | | Contractile force(40) | |
| | | | | | | Regulates hypertrophic | Adult |
| | | | | | | cardiomyopathy and | Heart |
| | | | | | | LVNC (38, 41, 42) | |
| | | | | | | Regulates inflammation | |
| | | | | | | (43, 44) | |
| | | | | | | Inhibited angiogenesis(18) | |
| | | | | | | Blood mutations (45) | |
| | | | | | | Velocardiofacial Syndrome (46) | |
| | | | | | | Muscle fiber development | Skeletal |
| | | | | | | and mitochondrial | Muscle |
| | | | | | | development (38) | |
| Mus | Smyd1 ID: | Transcript | NM_001160127.1 | SMYD1A | Smyd1a, | Ventricular formation | Fetal |
| musculus | 12180 | Variant 1 | | | m-Bop, | (2, 19, 47, 48) | Heart |
| (Mouse) | | | | | m-Bop1, | Regulates hypertrophic | Adult |
| | | | | | skm-BOP, | growth, fibrosis, and | Heart |
| | | | | | BOP | heart failure (49, 50) | |
| | | | | | | Regulates mitochondrial | |
| | | | | | | metabolism(51) | |
| | | | | | | Myofibrillogenesis (52, 53) | Skeletal |
| | | | | | | Myogenesis (48, 50, 54, 55) | Muscle |
| | | Transcript | NM_009762.2 | SMYD1B | Smyd1b, | Dispensable for hypertrophy | Neonatal |
| | | Variant 2 | | | m-Bop2 | (49) | Heart Cells |
| | | Transcript | G5E8R7° | SMYD1C | Smyd1c, | Mitochondrial metabolism | T-Cells |
| | | Variant 3° | | | t-BOP | (56) | |
| Rattus | Smyd1 ID: | Isoform X1¢ | XM_006236632.4 | SMYD1 | Smyd1 | Suppresses cardiomyocyte | Adult |
| norvegicus | 297333 | Isoform X2¢ | XM_017592535.2 | | | apoptosis (57, 58) | Heart |
| (Rat)^{†} | | Isoform X3¢ | XM_039107313.1 | | | Regulates cardiac | |
| | | | | | | energetics (58, 59) | |
| Danio | smyd1a ID: | Histone-lysine | NM_205540.2 | Smyd1a | Smyd1a, | Myofibrillogenesis | Fetal |
| rerio | 321245 | N-methyltransferase | | | SmyD1a, | (2, 5, 54, 60, 61) | Heart and |
| (Zebrafish) | | SMYD1a ¢ | | | Smyd1b | Thick filament assembly (17) | Skeletal |
| | | | | | | Sarcomere assembly (62, 63) | Muscle |
| | | | | | | Cold tolerance(64) | Skeletal |
| | | | | | | | Muscle |
| | smyd1b ID: | Isoform X1¢ | XM_021478292.1 | Smyd1b | Smyd1b, | Sarcomere assembly | Fetal |
| | 569027 | | | | SmyD1b, | (62, 63, 65, 66) | Heart |
| | | | | | Smyd1b tv1 | Sarcomere dysfunction (65) | and |
| | | | | | | Disruption of myofibril | Skeletal |
| | | | | | | organization (14, 67) | Muscle |
| | | | | | | Myofibrillogenesis (14, 68) | Skeletal |
| | | | | | | | Muscle |
| | | | | | Smyd1b | Myofibrillogenesis (68) | Skeletal |
| | | | | | Smyd1b tv2 | | Muscle |
| Paralichthys | smyd1a° | Smyd1a° | A0A411H9M0 | Smyd1a | Smyd1a | Metamorphosis (69) | Skeletal |
| olivaceus | | | | | | | Muscle |
| (Japanese | smyd1b ID: | Isoform X1¢ | XM_020099106.1 | Smyd1b | Smyd1b | Metamorphosis (69) | Skeletal |
| Flounder)* | 109637011 | Isoform X2¢ | XM_020099107.1 | | smyd1 | | Muscle |
| Siniperca | smyd1b ID: | Isoform X1¢ | XM_044197835.1 | Smyd1b | SmyD1a | Muscle development (49, 70) | Skeletal |
| chuatsi | 122876905 | Isoform X2¢ | XM_044197836.1 | | SmyD1b | | Muscle |
| (Chinese | | Isoform X3¢ | XM_044197837.1 | | | | |
| Perch)* | | | | | | | |
| Gallus | SMYD1 ID: | Isoform 1 | NM_204155.2 | SMYD1A | Smyd1a | Blood Vessel Development(71) | Fetal Heart |
| gallus | 373960 | Isoform 2 | NM_001397430.1 | SMYD1B | Smyd1b | Muscle Development (72) | Skeletal |
| (Chicken) | | | | | | | Muscle |

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these aspects are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nucleic Acid Sequences

Disclosed are nucleic acid sequences encoding SMYD1. SMYD1 refers to the human form of the protein. Disclosed are nucleic acid constructs comprising one or more of the disclosed nucleic acid sequences. For example, disclosed are nucleic acid constructs, wherein the nucleic acid construct comprises a nucleic acid sequence encoding SMYD1 SMYD1. In some aspects, the nucleic acid construct can be a vector. In some aspects, the vector can be a viral or non-viral vector as described herein.

In some aspects, the nucleic acid constructs can further comprise a promoter operably linked to the nucleic acid sequence encoding SMYD1. In some aspects, the promoter can be a cardiac specific promoter. For example, a cardiac specific promoter can be, but is not limited to, aMHC, cTNT, or MLC-2. In some aspects, the promoter can be a non-cardiac promoter. For example, a non-cardiac promoter can be, but is not limited to, CMV, CAG, Des, ANF, SLC, MCL, SPc5-12, or other ubiquitous promoters.

In some aspects, the promoters operably linked to the nucleic acid sequence encoding SMYD1 may be obtained from various sources, for example, the genomes of viruses such as polyoma. Simian Virus 40 (SV40), adenovirus, retroviruses, hepatitis B virus and most preferably cytomegalovirus, or from heterologous mammalian promoters, e.g., β-actin promoter. The early and late promoters of the SV40 virus are conveniently obtained as an SV40 restriction fragment, which also contains the SV40 viral origin of replication (Fiers et al., Nature, 273: 113 (1978) which is incorporated by reference herein in its entirety for viral promoters). The immediate early promoter of the human cytomegalovirus is conveniently obtained as a HindIII E restriction fragment (Greenway, P. J. et al., Gene 18: 355 360 (1982) which is incorporated by reference herein in its entirety for viral promoters). Of course, promoters from the host cell or related species also are useful herein, and can be used for tissue specific gene expression or tissues specific regulated gene expression. The cited references are incorporated herein by reference in their entirety for their teachings of promoters.

In some aspects, the nucleic acid constructs can further comprise an enhancer sequence. “Enhancer” generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Each of the cited references is incorporated herein by reference in their entirety for their teachings of enhancers. Furthermore, enhancers can be within an intron (Banerji, J. L. et al., Cell 33: 729 (1983)) as well as within the coding sequence itself (Osbome, T. F., et al., Mol. Cell Bio. 4: 1293 (1984)). Each of the cited references is incorporated herein by reference in their entirety for their teachings of potential locations of enhancers. They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100 270), the cytomegalovirus early promoter enhancer, the polyoma enhancer on the late side of the replication origin, and adenovirus enhancers.

In some aspects, the promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain aspects, the promoter and/or enhancer region can act as a constitutive promoter and/or enhancer to maximize expression of the region of the transcription unit to be transcribed. In certain constructs the promoter and/or enhancer region are active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

In some aspects, the nucleic acid sequence of human smyd1 is

AGTGTTAAATAACTGCCGCGCTGGCCTGACAGTCTCTGAGATGACAATAGGGAGAA
TGGAGAACGTGGAGGTCTTCACCGCTGAGGGCAAAGGAAGGGGTCTGAAGGCCAC
CAAGGAGTTCTGGGCTGCAGATATCATCTTTGCTGAGCGGGCTTATTCCGCAGTGGT
TTTTGACAGCCTTGTTAATTTTGTGTGCCACACCTGCTTCAAGAGGCAGGAGAAGCT
CCATCGCTGTGGGCAGTGCAAGTTTGCCCATTACTGCGACCGCACCTGCCAGAAGG
ATGCTTGGCTGAACCACAAGAATGAATGTTCGGCCATCAAGAGATATGGGAAGGTG
CCCAATGAGAACATCAGGCTGGCGGCGCGCATCATGTGGCGGGTGGAGAGAGAAG
GCACCGGGCTCACGGAGGGCTGCCTGGTGTCCGTGGACGACTTGCAGAACCACGTG
GAGCACTTTGGGGAGGAGGAGCAGAAGGACCTGCGGGTGGACGTGGACACATTCTT
GCAGTACTGGCCGCCGCAGAGCCAGCAGTTCAGCATGCAGTACATCTCGCACATCT
TCGGAGTGATTAACTGCAACGGTTTTACTCTCAGTGATCAGAGAGGCCTGCAGGCC
GTGGGCGTAGGCATCTTCCCCAACCTGGGCCTGGTGAACCATGACTGTTGGCCCAA
CTGTACTGTCATATTTAACAATGGCAATCATGAGGCAGTGAAATCCATGTTTCATAC
CCAGATGAGAATTGAGCTCCGGGCCCTAGGCAAGATCTCAGAAGGAGAGGAGCTG
ACTGTGTCCTATATTGACTTCCTCAACGTTAGTGAAGAACGCAAGAGGCAGCTGAA
GAAGCAGTACTACTTTGACTGCACATGTGAACACTGCCAGAAAAAACTGAAGGATG
ACCTCTTCCTGGGGGTGAAAGACAACCCCAAGCCCTCTCAGGAAGTGGTGAAGGAG
ATGATACAATTCTCCAAGGATACATTGGAAAAGATAGACAAGGCTCGTTCCGAGGG
TTTGTATCATGAGGTTGTGAAATTATGCCGGGAGTGCCTGGAGAAGCAGGAGCCAG
TGTTTGCTGACACCAACATCTACATGCTGCGGATGCTGAGCATTGTTTCGGAGGTCC
TTTCCTACCTCCAGGCCTTTGAGGAGGCCTCGTTCTATGCCAGGAGGATGGTGGACG
GCTATATGAAGCTCTACCACCCCAACAATGCCCAACTGGGCATGGCCGTGATGCGG
GCAGGGCTGACCAACTGGCATGCTGGTAACATTGAGGTGGGGCACGGGATGATCTG
CAAAGCCTATGCCATTCTCCTGGTGACACACGGACCCTCCCACCCCATCACTAAGGA
CTTAGAGGCCATGCGGGTGCAGACGGAGATGGAGCTACGCATGTTCCGCCAGAACG
AATTCATGTACTACAAGATGCGCGAGGCTGCCCTGAACAACCAGCCCATGCAGGTC
ATGGCCGAGCCCAGCAATGAGCCATCCCCAGCTCTGTTCCACAAGAAGCAATGAGG
ACTGCCCAGTGGAGGAGGGGCGATGTGGCTGGGGAGCTAGGGAGAGACTCTGGAG
GTGGTGGGTCTCTCGGGAGACCCCTAATGAGGAAGTTGAGGTAATGCTTAACATTG
TTGCTGTGAGAATTTACTGCCCTATGTTTCCCAGAGCCATTTTGGCTCAATTCAAGTC
TATTCAATTCAAGTTAACTCTAGCCCAGCCCAGATCAACTCCTCCTACAAATATTAT
TGGATGATAGGCCCTAGAACCCAATAAAGGAGCTCCAAATGTCGTTGGGTGGGGAA
GCAAAATGTAGAGAAACATTTAAAGCACACTGTAATAATAAATGCAATTATAAACT
ATATGGAGGAGGGTGCAGAGGAGGGAATGTGTCTGGTGTGTGATGTGTGTGTGTGC
AGTGGGGGTATCACAGAGAGTATGACATCTGAGTTGAGGGTAGCAGGTGCCTGGAG
TCTCAGGTGGCTGCTCACCCATCTGTGCAGGTGTCTCTGGGGCTGCTGGTCTCACCT
GTGGTCTGCAGTAGACACAATTGGCTGAGCAGGATATGTGATACTGTGTGGTTGGT
GTGGAGTTTTGAAGAAGGGGCTGTGTTTGGGCCACGTAGGCTCTACTCAGAGACCT
GAAACCACTTCAGAATGGTGCATATGTCGAAAGAGCTGGCTGGGGGCCTTGCCCAA
ACCAACTGAGGTCTTAAAGTCCAGGGAAAAAAAGTCTGGGTTCCAACTAGAATTCT
AGAAATATTTCTAGAACACACAGAGAGGGAATAAGTCCCTCTATCACCCTTATTAC
CAAGCCTTGTGGTTCCCTGTGATTTTAGATAATGTCTGATATTTTTCTGGCTATTTGC
CTAGTAGGATTTAAAAAATATTTTCAAAGTGAAGCTGAGAGAGAATCTTGGAAACA
CACATACCTGTTGATCATGGGCCCTGCAGAATTGGCCCTTGGGGGCTTTATTTGGTT
ACATGTGCCTGGGTGGTCTTTACCAGCTTAGACTCTATCATGGGCCCCCATGAAGCT
CCATTCTCAATACTGAATAATTATTACTTCCCTTGTTGAGTTTCTTTTTCTGTCATGC
CCTGGGGGCTTCTGCTCTTCTCACCAGAAAGAACATTTGAATCTGGATTCTTGTACA
CCTGGGTTAGACCCTGTTCAGAGGTGTGGCCAATTTATCCCGATCTCCTGGAAGGCT
GTTGTGATTTCCATCTAAGAAATGAGGGTCTTGAGAATCAACCAGTCCCAAGATTA
GCCTGTTATCCTGTTATCTACTGAGACCCCAAATTTCTCACCAATGTTTTGGGAGAT
CCTGGAAAAGATCCCTTCAGTTTGGGGTGTCACCAAGACTTCTACACAACCCAGGA
CTACCATTGACCTCAGAGCTGTACCCCACATCTTGAAGTAAATTGATCCCACCAGGT
CCCACGTTTGTTATCTCTGCCTAAATGTTAGCTTCTCCATCCTCACCACATGATGACC
TGCTGTGTCCCTCTGAGCACTACCCAGTGGCTGAAAACTCTGCAAATGGGCCACACT
TTTGCAAAATACTTGTATCTGACACTTAGGTCTTGTTTGAAGAATTTCCTTTCTGGAA
GGTTTTACAAGAAGACTGATAGTCTTTCAAGCCCCCACATCACAGGCTTAGGGACG
GCACTAACTTTCTCCCAGGGATCTAACTGGCTAGTTCAAATTATCACTCTTTTACCTT
CATATAAAATGTCTCCCCCAAACCTTTTTCCCTTCTTTGTCATTGTTATCTGCTAAGC
CCCTGGTCATTTCCCCATATTCGTAGTCTTTTTTTCCATCCTATCTTTCTAATATTTGT
TGTCTTTAACAAACTGTGTTCTGTGTCTGTGCTCCTCCTTCCCTCTCAGACCACTGGA
ATGCAAGTCCTTCTTCCCTTTGGAATGTACTCTGGATCCCTTCCCCTGCTTTGACCCC
CAGACTTTGCTCCATCTATTATTGCTTCTCCATCCTGGATCCTTGACATTTGTCACCC
CACTGGCCTTCTCAGGTGCAATCAGTAAAAATGCTGAGAACTCTTGGATCTTAATCT
TCATGACTGAGTTTTTTTTAGTTGTATAGTTATCATCTGCCTTTCTTCACTTTGCATTT
CTTCTTGAATCCATTGCAGATTGACTTCCACTCCCACTCCTTCACTAAAAGGGCTCTT
ACCAAGATCAAATCTAATGGGTACATTTTAGTTCCTATGTGATTTGGCCTTTCGATG
TCAATCATCACTCCCAGCCATTGATTTTGGTGACCCACTTCCCTGTGATGATCTTCTG
ATCTAGTTTCTCAGGTTCCTTCGCTGGTCCTTTTTCTTTCCCTGCCCCTGACATATTG
ACATTTCCTGGAGTTGGTTTTGTCCTTGATTCATTCTCATGTCATTCTGCACACAGTC
TCTGCATGAACTCAGGCAGACCCTTCATTTAATGACCACCTTAGGGCTGATGATTCT
CAAATCTGTATTCCCCGATCTTGCATTTGAGCTCCAGCCCCACTCATCCTCTCGGAT
GTTCTGCAGGCCCAGCAAACTCATCATGTCCAAAGTGAAACTTTTTCTCTTTCCTGT
CTCCTCTCCTCTGATCTGTTCTTTCTTGGAACACCACCCAAGAACGTCACCTCCTCCA
TCAGATTGTGAGCTCCTGGAGGGCAGGAGCTGTGTCCTTCTATTCATCTTCCTATCC
CCAGAACCTTGCACAGATCCTGGAATGTGGTAGGTGCTCAGTAAATGTGTGTTGAA
TAAATGAATGAATGAATGAACAAATGAATGAATTTGCTTACTTCAAGGCAAAAGAA
CCATGAAACTGTATTTTGAGTTTCTATGTTATAGCAGTCAGCAAATCCTATTAAATA
CTTTGTGTTTCCAAGCAAA (Accession No. NM_198274.4).

In some aspects, the nucleic acid sequence encoding human SMYD1 encodes the amino acid sequence of

MTIGRMENVEVFTAEGKGRGLKATKEFWAADIIFAERAYSAVVFDSLVN
FVCHTCFKRQEKLHRCGQCKFAHYCDRTCQKDAWLNHKNECSAIKRYGK
VPNENIRLAARIMWRVEREGTGLTEGCLVSVDDLQNHVEHFGEEEQKDL
RVDVDTFLQYWPPQSQQFSMQYISHIFGVINCNGFTLSDQRGLQAVGVG
IFPNLGLVNHDCWPNCTVIFNNGNHEAVKSMFHTQMRIELRALGKISEG
EELTVSYIDFLNVSEERKRQLKKQYYFDCTCEHCQKKLKDDLFLGVKDN
PKPSQEVVKEMIQFSKDTLEKIDKARSEGLYHEVVKLCRECLEKQEPVF
ADTNIYMLRMLSIVSEVLSYLQAFEEASFYARRMVDGYMKLYHPNNAQL
GMAVMRAGLTNWHAGNIEVGHGMICKAYAILLVTHGPSHPITKDLEAMR
VQTEMELRMFRQNEFMYYKMREAALNNQPMQVMAEPSNEPSPALFHKKQ
(Accession No. NP_938015.1)

In some aspects, the nucleic acid sequence of mouse smyd1 is

AGGAGCTAAATAGCAGCAAGGACAAGAGGCTTGGCTCAGTGTGAGCGAGCCACCT
CGAAGGCCTGACAGACTCTGAGATGACAATAGGCAGCATGGAGAACGTGGAGGTC
TTCACTTCCGAGGGCAAAGGCAGAGGTCTGAAGGCTACGAAGGAGTTCTGGGCTGC
GGATGTCATCTTTGCGGAGAGGGCTTATTCTGCAGTGGTTTTTGACAGCCTCATTAA
CTTCGTGTGCCACACCTGCTTCAAGAGGCAGGAGAAGCTCCATCGCTGCGGGCAGT
GCAAGTTCGCCCATTACTGCGACCGCACGTGCCAGAAGGATGCTTGGCTGAATCAC
AAGAACGAGTGCGCTGCCATCAAGAAATATGGGAAAGTGCCCAACGAGAACATCA
GGCTGGCCGCCCGCATCATGTGGCGGGTGGAGAGAGAGGGCACTGGGCTCACAGA
GGGCTGCCTGGTGTCCGTGGATGACTTACAGAACCACGTGGAGCACTTTGGGGAGG
AGGAGCAGAAGGAACTCCGAGTAGACGTGGACACGTTCTTGCAGTACTGGCCACCA
CAGAGCCAGCAGTTCAGCATGCAGTATATCTCACACATCTTTGGTGTGATCAACTGC
AACGGTTTCACTCTCAGTGACCAGAGAGGGCTACAGGCAGTAGGTGTGGGCATCTT
CCCCAACCTGGGCCTGGTGAACCATGATTGCTGGCCAAACTGCACTGTCATATTCAA
CAATGGCAATCATGAGGCAGTGAAATCCATGTTTCACACGCAGATGAGGATTGAGC
TCCGGGCCCTGGGCAAGATCTCAGAAGGCGAGGAGCTGACTGTGTCCTACATAGAC
TTCCTGCACCTCAGTGAGGAGCGCAGGCGGCAGCTGAAGAAACAGTACTACTTTGA
CTGCTCCTGTGAGCACTGCCAGAAGGGGCTGAAGGACGACCTCTTCCTGGCAGCGA
AGGAAGACCCCAAGCCCTCCCAGGAAGTGGTGAAGGAGATGATACAATTCTCAAA
GGATACACTGGAAAAAATAGACAAGGCTCGCTCCGAGGGTTTGTATCACGAGGTTG
TGAAGCTGTGTCGGGAGTGCCTGGAGAAGCAGGAGCCAGTGTTCGCCGACACCAAC
CTCTACGTGCTTCGGCTGCTCAGCATTGCATCAGAGGTCCTCTCCTACCTCCAGGCC
TATGAGGAGGCCTCACATTATGCCAGGAGGATGGTTGATGGCTACATGAAACTCTA
CCACCATAACAATGCCCAACTGGGCATGGCTGTGATGAGGGCAGGGCTTACCAACT
GGCATGCTGGTCACATCGAAGTGGGGCATGGGATGATCTGCAAAGCCTATGCTATT
CTCCTGGTGACCCATGGACCCTCCCACCCTATCACCAAAGACTTAGAGGCCATGCG
GATGCAGACAGAGATGGAGCTGCGTATGTTCCGCCAAAACGAATTCATGTATCACA
AGATGCGAGAGGCTGCCCTGAACAACCAGCCCATGCAGGTCATGGCTGAGCCTAGC
AATGAACCAGCCCCCGCTCTGTTCCATAAGAAGCAGTGAGGACCTTGTTGGAGGGT
GGGGGGGCTGTGGCTGGGGAGCTGAGGAAATGACTACTCTGTGTCCTCCACTGTCG
TTTTGGTTCAATTCAATTCAGTTTAGTTCTCTTTCATTTCAGACCAACTCTTCCTACA
TGTGTCAGGTGCTAGATCCCACCATCTATTTAATAAGGGAGCCCCAAATATAATTGT
GCTAGAAAGAAAAATGTAAACAAACAAGGCACAGCATAATAGTAAATGCCATGGT
GAGCTCTATGCAGAGGGGTTTGTTTAGGGGAGGTGAGTTTGTGTGTGTATCGGGTG
GGGGTAACACCTGAGCTAAGAGTGGCAGGTACCTAGAATCCCAGGTGACTGCTTGT
GTATCTGTGTAGGCATCTCTGGGGCTGCTGGTCTCATCTACAGTTATGAATATTTAG
GTTTTGCAGACTGGGGTGGGTGATGTCCTGAATCCTCAGGCCAAAAGTGTTTGACCC
TTTGAGATATACTCAGTGATGCCTAGAAAGATGCAGGGATCTTGGGATCCTGTGGTC
TTTACTCCTAATCAGAGCTTGAAGCATAACTTTAAAATAACTCTAGAACACACATAG
AAGGAATTTTTCCCTCCCTGGTCCTACCCACCATGCCTGTAGCTCTTTATGATGTTGA
AGAAAAGCTGAGTTTTTCCTGGCCATTGACTTGTAAAGCTGACTGTGCATGCCTGCC
AGTGATGGACCCATCAGAAGTGGTGTGTAGGGGCTTCCAAGACTGAGAAGTCTGCC
TCAGCCTAGGCTTCATCATTGGCCCAATGAGGCTCTACTCATTGTATTGAGTGACTG
TTAGTTCCTCTGCTTAATTTCTTTATCCTGTCTGTGCTCTGGGGGCTTCTCCCCTTTCG
TAAGGCAGTTCACCTGGCCTGGATCTCATTGGAGAGTGGCCAGTTTGCTCTCATTTA
CTTGGAGGCTGACTTCCAGCTGAGAAATGAAGGCCCAAGAAGTGTCTTTTAGTTTCT
GGAACCCCAACTTTCACACCAACATGATGGGAAATCATGAAGTAAATTCTTTTGGTT
GAGGGTGTCACCAAAACTTCTACGAAACCTTGTGCTACCACTGACATCTCCCAGAA
CTCTAACATCTTGAAGTCACATGATTCCCACTAAACTGCAATTTATTTCTGCCTAAA
ATGTTAGTTTCTCCATCCCCAGCCTTTCATCCCAGAACAGTACCCTACCTGCCCTTGA
GCCCTGCCCAGCCTCTGAGGCCTATGATGCGGGCACCTCTTTGTAGAATATTAGCAC
TGACCCTTGGTTCTTTTTGAGGATTTGTTCTTCTCTAGAAGGCTTTATGAAGACTGAC
AGTGAAGACCATGCTCAGTATAAACTTACAATGGGGGGGGGGGGGGGATGACAGA
GTGGATCCTTAAAATGTTAAAGGAGTTGTGGCTTAACTGGAGACTACTCACAAACC
CTGAGAGCCACAGTAAACAAGCCTAAGAGTTCCACGCACCCCCCTCACCATCTGCA
TCCACCCCATTTTGGTGAACCAGAGCACTAATCAGACATCTGTCATTGCCTGGTGAC
TCTATTAGGGACTGCCTCTTTAAAGATCTGAGGAAGAGAACTATGGTGAGGCCTTG
ACCATGGCCAGAAGGATTTTCCTACAAAGCACTTTATTTGCAGAGTGTAAATTCATC
ATTAAGTACCTGCCGTCTCCTGGTCAAGTTCAAACTCCAAAGTTAAAAGATTCACAG
CAGATGCTCTGTTCTCTCTCTGTGTCCTCTCGTGTATGCCCATTTTCACTTTACCTGCT
TGCAAAATAAATTCCCTTGGATGCTCTCGGAAAAAAAAAAAAAAAAAAA (Accession
No. NM_001160127.1)

In some aspects, the nucleic acid sequence encoding mouse SMYD1 encodes the amino acid sequence of

MTIGSMENVEVFTSEGKGRGLKATKEFWAADVIFAERAYSAVVFDSLIN
FVCHTCFKRQEKLHRCGQCKFAHYCDRTCQKDAWLNHKNECAAIKKYGK
VPNENIRLAARIMWRVEREGTGLTEGCLVSVDDLQNHVEHFGEEEQKEL
RVDVDTFLQYWPPQSQQFSMQYISHIFGVINCNGFTLSDQRGLQAVGVG
IFPNLGLVNHDCWPNCTVIFNNGNHEAVKSMFHTQMRIELRALGKISEG
EELTVSYIDFLHLSEERRRQLKKQYYFDCSCEHCQKGLKDDLFLAAKED
PKPSQEVVKEMIQFSKDTLEKIDKARSEGLYHEVVKLCRECLEKQEPVF
ADTNLYVLRLLSIASEVLSYLQAYEEASHYARRMVDGYMKLYHHNNAQL
GMAVMRAGLTNWHAGHIEVGHGMICKAYAILLVTHGPSHPITKDLEAMR
MQTEMELRMFRQNEFMYHKMREAALNNQPMQVMAEPSNEPAPALFHKKQ
(Accession No. NP_001153599.1)

In some aspects, a nucleic acid construct comprises the nucleic acid sequence of

(SEQ ID NO: 1)
cctgcaggca gctgcgcgct cgctcgctca ctgaggccgc ccgggcgtcg ggcgaccttt ggtcgcccgg cctcagtgag
cgagcgagcg cgcagagagg gagtggccaa ctccatcact aggggttcct gcggccgcac gcgtctagtt attaatagta
atcaattacg gggtcattag ttcatagccc atatatggag ttccgcgtta cataacttac ggtaaatggc ccgcctggct
gaccgcccaa cgacccccgc ccattgacgt caataatgac gtatgttccc atagtaacgt caatagggac tttccattga
cgtcaatggg tggagtattt acggtaaact gcccacttgg cagtacatca agtgtatcat atgccaagta cgccccctat
tgacgtcaat gacggtaaat ggcccgcctg gcattatgcc cagtacatga ccttatggga ctttcctact tggcagtaca
tctacgtatt agtcatcgct attaccatgg tgatgcggtt ttggcagtac atcaatgggc gtggatagcg gtttgactca
cggggatttc caagtctcca ccccattgac gtcaatggga gtttgttttg caccaaaatc aacgggactt tccaaaatgt
cgtaacaact ccgccccatt gacgcaaatg ggcggtaggc gtgtacggtg ggaggtctat ataagcagag ctcgtttagt
gaaccgtcag atcgcctgga gacgccatcc acgctgtttt gacctccata gaagacaccg ggaccgatcc agcctccgcg
gattcgaatc ccggccggga acggtgcatt ggaacgcgga ttccccgtgc caagagtgac gtaagtaccg cctatagagt
ctataggccc acaaaaaatg ctttcttctt ttaatatact tttttgttta tcttatttct aatactttcc
ctaatctctt tctttcaggg
caataatgat acaatgtatc atgcctcttt gcaccattct aaagaataac agtgataatt tctgggttaa ggcaatagca
atatttctgc atataaatat ttctgcatat aaattgtaac tgatgtaaga ggtttcatat tgctaatagc agctacaatc
cagctaccat tctgctttta ttttatggtt gggataaggc tggattattc tgagtccaag ctaggccctt
ttgctaatca tgttcatacc
tcttatcttc ctcccacagc tcctgggcaa cgtgctggtc tgtgtgctgg cccatcactt tggcaaagaa ttgggattcg
aacatcgatt gaattccccg gggatccttc gacggcgcgc cgaattccaa gaggcttggc tcagtgtgag cgagccacct
cgaaggcctg acagactctg agatgacaat aggcagcatg gagaacgtgg aggtcttcac ttccgagggc aaaggcagag
gtctgaaggc tacgaaggag ttctgggctg cggatgtcat ctttgcggag agggcttatt ctgcagtggt ttttgacagc
ctcattaact tcgtgtgcca cacctgcttc aagaggcagg agaagctcca tcgctgcggg cagtgcaagt tcgcccatta
ctgcgaccgc acgtgccaga aggatgcttg gctgaatcac aagaacgagt gcgctgccat caagaaatat gggaaagtgc
ccaacgagaa catcaggctg gccgcccgca tcatgtggcg gctggagaga gagggcactg ggctcacaga gggctgcctg
gtgtccgtcg atgacttaca gaaccacgtg gagcactttg gggaggagga gcagaaggaa ctccgagtag acgtggacac
gttcttgcag tactggccac cacagagcca gcagttcagc atgcagtata tctcacacat ctttggtgtg atcaactgca
acggtttcac tctcagtgac cagagagggc tacaggcagt aggtgtgggc atcttcccca acctgggcct ggtgaaccat
gattgctggc caaactgcac tgtcatattc aacaatggca atcatgaggc agtgaaatcc atgtttcaca cgcagatgag
gattgagctc cgggccctgg gcaagatctc agaaggcgag gagctgactg tgtcctacat agacttcctg cacctcagtg
aggagcgcag gcggcagctg aagaaacagt actactttga ctgctcctgt gagcactgcc agaaggggct gaaggacgac
ctcttcctgg cagcgaagga agaccccaag ccctcccagg aagtggtgaa ggagatgata caattctcaa aggatacact
ggaaaaaata gacaaggctc gctccgaggg tttgtatcac gaggttgtga agctgtgtcg ggagtgcctg gagaagcagg
agccagtgtt cgccgacacc aacctctacg tgcttcggct gctcagcatt gcatcagagg tcctctccta cctccaggcc
tatgaggagg cctcacatta tgccaggagg atggttgatg gctacatgaa actctaccac cataacaatg cccaactggg
catggctgtg atgagggcag ggcttaccaa ctggcatgct ggtcacatcg aagtggggca tgggatgatc tgcaaagcct
atgctattct cctggtgacc catggaccct cccaccctat caccaaagac ttagaggcca tgcggatgca gacagagatg
gagctgcgta tgttccgcca aaacgaattc atgtatcaca agatgcgaga ggctgccctg aacaaccagc ccatgcaggt
catggctgag cctagcaatg aaccagcccc cgctctgttc cataagaagc agtgaggacc ttgttggagg gtgggggggc
tgtggctggg gagctgagga aatgactact ctgtgtcctc cactgtcgtt ttggttcaat tcaattcagt
ttagttctct ttcatttcag
accaactctt cctacatgtg tcaggtgcta gatcccacca tctatttaat aagggagccc caaatataat
tgtgctagaa agaaaaatgt
aaacaaacaa ggcacagcat aatagtaaat gccatggtga gctctatgca gaggggtttg tttaggggag gtgagtttgt
gtgtgtatcg ggtgggggta acacctgagc taagagtggc aggtacctag aatcccaggt gactgcttgt gtatctgtgt
aggcatctct ggggctgctg gtctcatcta cagttatgaa tatttaggtt ttgcagactg gggtgggtga tgtcctgaat
cctcaggcca aaagtgtttg accctttgag atatactcag tgatgcctag aaagatgcag ggatcttggg atcctgtggt
ctttactcct aatcagagct tgaagcataa ctttgctgct cgagagatct acgggtggca tccctgtgac
ccctccccag tgcctctcct
ggccctggaa gttgccactc cagtgcccac cagccttgtc ctaataaaat taagttgcat cattttgtct
gactaggtgt ccttctataa
tattatgggg tggagggggg tggtatggag caaggggcaa gttgggaaga caacctgtag ggcctgcggg gtctattggg
aaccaagctg gagtgcagtg gcacaatctt ggctcactgc aatctccgcc tcctgggttc aagcgattct
cctgcctcag cctcccgagt
tgttgggatt ccaggcatgc atgaccaggc tcagctaatt tttgtttttt tggtagagac ggggtttcac
catattggcc aggctggtct
ccaactccta atctcaggtg atctacccac cttggcctcc caaattgctg ggattacagg cgtgaaccac tgctcccttc
cctgtccttc tgattttgta ggtaaccacg tgcggaccga gcggccgcag gaacccctag tgatggagtt ggccactccc
tctctgcgcg ctcgctcgct cactgaggcc gggcgaccaa aggtcgcccg acgcccgggc tttgcccggg cggcctcagt
gagcgagcga gcgcgcagct gcctgcaggg gcgcctgatg cggtattttc tccttacgca ctgtgcggt atttcacacc
gcatacgtca aagcaaccat agtacgcgcc ctgtagcggc gcattaagcg cggcgggtgt ggtggttacg cgcagcgtga
ccgctacact tgccagcgcc ctagcgcccg ctcctttcgc tttcttccct tcctttctcg ccacgttcgc cggctttccc
cgtcaagctc taaatcgggg gctcccttta gggttccgat ttagtgcttt acggcacctc gaccccaaaa aacttgattt
gggtgatggt tcacgtagtg ggccatcgcc ctgatagacg gtttttcgcc ctttgacgtt ggagtccacg
ttctttaata gtggactctt
gttccaaact ggaacaacac tcaaccctat ctcgggctat tcttttgatt tataagggat tttgccgatt tcggcctatt
ggttaaaaaa tgagctgatt taacaaaaat ttaacgcgaa ttttaacaaa atattaacgt ttacaatttt atggtgcact
ctcagtacaa tctgctctga tgccgcatag ttaagccagc cccgacaccc gccaacaccc gctgacgcgc cctgacgggc
ttgtctgctc ccggcatccg cttacagaca agctgtgacc gtctccggga gctgcatgtg tcagaggttt tcaccgtcat
caccgaaacg cgcgagacga aagggcctcg tgatacgcct atttttatag gttaatgtca tgataataat ggtttcttag
acgtcaggtg gcacttttcg gggaaatgtg cgcggaaccc ctatttgttt atttttctaa atacattcaa
atatgtatcc gctcatgaga
caataaccct gataaatgct tcaataatat tgaaaaagga agagtatgag tattcaacat ttccgtgtcg
cccttattcc cttttttgcg
gcattttgcc ttcctgtttt tgctcaccca gaaacgctgg tgaaagtaaa agatgctgaa gatcagttgg gtgcacgagt
gggttacatc gaactggatc tcaacagcgg taagatcctt gagagttttc gccccgaaga acgttttcca
atgatgagca cttttaaagt
tctgctatgt ggcgcggtat tatcccgtat tgacgccggg caagagcaac tcggtcgccg catacactat
tctcagaatg acttggttga
gtactcacca gtcacagaaa agcatcttac ggatggcatg acagtaagag aattatgcag tgctgccata accatgagtg
ataacactgc ggccaactta cttctgacaa cgatcggagg accgaaggag ctaaccgctt ttttgcacaa catgggggat
catgtaactc gccttgatcg ttgggaaccg gagctgaatg aagccatacc aaacgacgag cgtgacacca cgatgcctgt
agcaatggca acaacgttgc gcaaactatt aactggcgaa ctacttactc tagcttcccg gcaacaatta atagactgga
tggaggcgga taaagttgca ggaccacttc tgcgctcggc ccttccggct ggctggttta ttgctgataa atctggagcc
ggtgagcgtg ggtctcgcgg tatcattgca gcactggggc cagatggtaa gccctcccgt atcgtagtta tctacacgac
ggggagtcag gcaactatgg atgaacgaaa tagacagatc gctgagatag gtgcctcact gattaagcat tggtaactgt
cagaccaagt ttactcatat atactttaga ttgatttaaa acttcatttt taatttaaaa ggatctaggt
gaagatcctt tttgataatc
tcatgaccaa aatcccttaa cgtgagtttt cgttccactg agcgtcagac cccgtagaaa agatcaaagg atcttcttga
gatccttttt ttctgcgcgt aatctgctgc ttgcaaacaa aaaaaccacc gctaccagcg gtggtttgtt tgccggatca
agagctacca actctttttc cgaaggtaac tggcttcagc agagcgcaga taccaaatac tgtccttcta
gtgtagccgt agttaggcca
ccacttcaag aactctgtag caccgcctac atacctcgct ctgctaatcc tgttaccagt ggctgctgcc
agtggcgata agtcgtgtct
taccgggttg gactcaagac gatagttacc ggataaggcg cagcggtcgg gctgaacggg gggttcgtgc acacagccca
gcttggagcg aacgacctac accgaactga gatacctaca gcgtgagcta tgagaaagcg ccacgcttcc cgaagggaga
aaggcggaca ggtatccggt aagcggcagg gtcggaacag gagagcgcac gagggagctt ccagggggaa acgcctggta
tctttatagt cctgtcgggt ttcgccacct ctgacttgag cgtcgatttt tgtgatgctc gtcagggggg cggagcctat
ggaaaaacgc cagcaacgcg gcctttttac ggttcctggc cttttgctgg ccttttgctc acatgt.

The italicized sequence is the CMV promoter. The underlined sequence is the mSMYD1 gene.

Disclosed are nucleic acid constructs comprising a nucleic acid sequence, wherein the nucleic acid sequence comprises an aMHC promoter sequence, a human SMYD1 gene sequence, a FLAG sequence, a WPRE sequence, and a polyA sequence, wherein the nucleic acid sequence is flanked by ITR sequences. Disclosed are nucleic acid constructs comprising a nucleic acid sequence, wherein the nucleic acid sequence comprises an aMHC promoter sequence, a mounse SMYD1a gene sequence, a FLAG sequence, a WPRE sequence, and a polyA sequence, wherein the nucleic acid sequence is flanked by ITR sequences. In some aspects, the ITR sequences are considered part of the nucleic acid sequence. In some aspects, the ITR sequences can be from AAV9.

Disclosed are nucleic acid constructs comprising a nucleic acid sequence, wherein the nucleic acid sequence comprises an aMHC promoter sequence, a human SMYD1 gene sequence, a GFP sequence, a WPRE sequence, and a polyA sequence, wherein the nucleic acid sequence is flanked by ITR sequences. Disclosed are nucleic acid constructs comprising a nucleic acid sequence, wherein the nucleic acid sequence comprises an αMHC promoter sequence, a mounse SMYD1a gene sequence, a GFP sequence, a WPRE sequence, and a polyA sequence, wherein the nucleic acid sequence is flanked by ITR sequences. In some aspects, the ITR sequences are considered part of the nucleic acid sequence. In some aspects, the ITR sequences can be from AAV9.

### C. Vectors

Disclosed are vectors comprising one or more of the disclosed nucleic acid sequences or nucleic acid constructs. In some instances, the vector can be selected from the group consisting of viral or non-viral vector. In some aspects, the vector can be a nucleic acid based vector or non-nucleic acid based vector.

In some aspects, a nucleic acid construct and a vector can be the same thing. Disclosed are vectors comprising a nucleic acid sequence, wherein the nucleic acid sequence comprises an αMHC promoter sequence, a human SMYD1 gene sequence, a FLAG sequence, a WPRE sequence, and a polyA sequence, wherein the nucleic acid sequence is flanked by ITR sequences. Disclosed are vectors comprising a nucleic acid sequence, wherein the nucleic acid sequence comprises an aMHC promoter sequence, a mounse SMYD1a gene sequence, a FLAG sequence, a WPRE sequence, and a polyA sequence, wherein the nucleic acid sequence is flanked by ITR sequences. In some aspects, the ITR sequences are considered part of the nucleic acid sequence. In some aspects, the ITR sequences can be from AAV9.

Disclosed are vectors comprising a nucleic acid sequence, wherein the nucleic acid sequence comprises an aMHC promoter sequence, a human SMYD1 gene sequence, a GFP sequence, a WPRE sequence, and a polyA sequence, wherein the nucleic acid sequence is flanked by ITR sequences. Disclosed are vectors comprising a nucleic acid sequence, wherein the nucleic acid sequence comprises an αMHC promoter sequence, a mounse SMYD1a gene sequence, a GFP sequence, a WPRE sequence, and a polyA sequence, wherein the nucleic acid sequence is flanked by ITR sequences. In some aspects, the ITR sequences are considered part of the nucleic acid sequence. In some aspects, the ITR sequences can be from AAV9.

In some instances, the vector can comprise a promoter, such as the cardiac-specific promoters or non-cardiac specific promoters described herein operably linked to a nucleic acid sequence encoding SMYD1.

In some aspects, the vector can be an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” can be used interchangeably, as a plasmid is a commonly used form of vector. Moreover, the invention is intended to include other vectors which serve equivalent functions.

In some aspects, the vector can be a viral vector. For example, the viral vector can be a lentiviral vector. In some aspects, the vector can be a non-viral vector, such as a DNA based vector.

In some aspects, a vector can be used for nucleic acid delivery, in an in vitro setting, in vivo setting, or any combination thereof. In some cases, the vector can be targeted to but may not be limited to a mammal, or a specific organ, or a specific cell, or any combination thereof. The vector can comprise any composition described herein. In some cases, the vector can comprise more than one composition. In some cases, the vector can be comprised of a liposome, a nanoparticle or any combination thereof. The liposome can include but may not be limited to unilamellar liposome, multilamellar liposome, archaeosome, niosome, novasome, cryptosome, emulsome, vesosome, or a derivative of any of these, or any combination thereof. The nanoparticle can include but may not be limited to biopolymeric nanoparticle, alginate nanoparticle, xanthan gum nanoparticle, cellulose nanoparticle, dendrimer, polymeric micelle, polyplexed, inorganic nanoparticle, nanocrystal, metallic nanoparticle, quantum dot, protein nanoparticle, polysaccharide nanoparticle, or a derivative of any of these, or any combination thereof. In some cases, the vector can be an RNA viral vector which can include but may not be limited to a retrovirus, lentivirus, coronavirus, alphavirus, flavivirus, rhabdovirus, morbillivirus, picornavirus, coxsackievirus, or picornavirus or portions of any of these, or fragments of any of these, or any combination thereof. In some cases, the vector can be a DNA viral vector which can include but may not be limited to an adeno-associated viral (AAV) vector, adenovirus, hybrid adenoviral system, hepadnavirus, parvovirus, papillomavirus, polyomavirus, herpesvirus, poxvirus, a portion of any of these, or a fragment of any of these, or any combination thereof.

A vector can be employed to deliver the engineered oligonucleotide, the nucleic acid construct, or any combination thereof. A vector can comprise DNA, such as double stranded DNA or single stranded DNA. A vector can comprise RNA. In some cases, the nucleic acid sequence can comprise a base modification. The vector can comprise a recombinant vector. The vector can be a vector that is modified from a naturally occurring vector. The vector can comprise at least a portion of a non-naturally occurring vector. Any vector can be utilized.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences to improve expression from, or stability of, the construct.

Cre Recombinase is a Type I topoisomerase from bacteriophage P1 that catalyzes the site-specific recombination of DNA between loxP sites (Abremski, K. and Hoess, R. (1984) J. Biol. Chem., 259, 1509-1514, which is incorporated herein by reference in its entirety for its teachings of Cre Recombinase structure and function ). The enzyme requires no energy cofactors and Cre-mediated recombination quickly reaches equilibrium between substrate and reaction products (Abremski, K. et al. (1983) Cell, 32, 1301-1311, which is incorporated herein by reference in its entirety for its teachings of the mechanism of action of Cre Recombinase). The loxP recognition element is a 34 base pair (bp) sequence comprised of two 13 bp inverted repeats flanking an 8 bp spacer region which confers directionality (Metzger. D. and Feil, R. (1999) Curr. Opin. Biotechnol., 10, 470-476, which is incorporated herein by reference in its entirety for its teachings of loxP recognition elements and teir role in Cre Recombinase action). Recombination products depend on the location and relative orientation of the loxP sites. Two DNA species containing single loxP sites can be fused. DNA between directly repeated loxP sites will be excised in circular form while DNA between opposing loxP sites will be inverted with respect to external sequences.

Expression of nucleic acid sequences operably linked to the transcriptional control elements in the gene transfer constructs described herein can also be regulated by Cre recombinase.

i. Viral and Non-Viral Vectors

In some aspects, the delivery vehicle or vector used to deliver SMYD1 to a cell or subject can be viral or non-viral. Examples of non-viral vectors include, but are not limited to, plasmids, liposomes, microcapsules, nanoparticles, lipid nanoparticles (LNPs), highly branched poly(β-amino ester) (HPAE), single-chain cyclic polymer (SCKP), poly(amidoamine) (PAMAM) dendrimers, and polyethyleneimine (PEI).

In some cases, the vector can comprise a viral vector, a liposome, a nanoparticle, an exosome, an extracellular vesicle, or any combination thereof. In some cases, a viral vector can comprise an adenoviral vector, an adeno-associated viral vector (AAV), a lentiviral vector, a retroviral vector, a portion of any of these, or any combination thereof. In some cases, a nanoparticle vector can comprise a polymeric-based nanoparticle, an aminolipid based nanoparticle, a metallic nanoparticle (such as gold-based nanoparticle), a portion of any of these, or any combination thereof. In some cases, a vector can comprise an AAV vector. A vector can be modified to include a modified VP1 protein (such as an AAV vector modified to include a VP1 protein). An AAV can comprise a serotype—such as an AAV I serotype, an AAV2 serotype, AAV3 serotype, an AAV4 serotype, AAV5 serotype, an AAV6 serotype, AAV7 serotype, an AAV8 serotype, an AAV9 serotype, a derivative of any of these, or any combination thereof

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding SMYD1.

The “control elements” present in an expression vector are those non-translated regions of the vector-enhancers, promoters. 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla. Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osbome, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also wavs to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some aspects, the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P. J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukarvotic control to convey resistance to the appropriate drug G418 or neomycin (geneticin), xgpt (mycophenolic acid) or hygromycin, respectively. Others include the neomycin analog G418 and puramycin.

As used herein, plasmid (e.g., non-viral vectors) or viral vectors are agents that transport the disclosed nucleic acids, such as a nucleic acid sequence capable of encoding SMYD1 into the cell without degradation and include a promoter yielding expression of the gene in the cells into which it is delivered. In some aspects, the nucleic acid sequences disclosed herein are derived from either a virus or a retrovirus. Viral vectors are, for example, Adenovirus, Adeno-associated virus, Herpes virus, Lentivirus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232. Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987): Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993): Caillaud. Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985): Seth, et al., J. Virol. 51:650-655 (1984); Seth, et al., Mol. Cell. Biol., 4:1528-1533 (1984): Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector. In some aspects, the AAV vector can be any one of AAV1-9.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a nucleic acid sequences, nucleic acid constructs, or vectors and a cationic liposome can be administered to the blood, to a target organ, or inhaled into the respiratory tract to target cells of the respiratory tract. For example, a composition comprising a nucleic acid sequences, nucleic acid constructs, or vectors described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### D. Cells

Disclosed are cells comprising one or more of the nucleic acid sequences, nucleic acid constructs, or vectors disclosed herein. In some aspects, the cells are prokaryotic or eukaryotic. In some aspects, the cells are mammalian cells.

In some aspects, the cells can be a cell line.

### E. Compositions

Disclosed are compositions comprising any of the disclosed nucleic acid sequences, nucleic acid constructs, or vectors. In some instances, disclosed are compositions comprising a vector comprising one or more of the disclosed nucleic acid constructs, wherein the nucleic acid construct comprises a nucleic acid sequence encoding SMYD1.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the nucleic acid sequences, nucleic acid constructs, or vectors of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed nucleic acid sequences, nucleic acid constructs, or vectors can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In some aspects, the compositions disclosed herein can also comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a vector and a cationic liposome can be administered to the blood, to a target organ, or inhaled into the respiratory tract to target cells of the respiratory tract. For example, a composition comprising a nucleic acid sequences, nucleic acid constructs, or vectors described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed nucleic acid sequences, nucleic acid constructs, or vectors described herein, or a pharmaceutically acceptable salt or solvate thereof, and a pharmaceutically acceptable carrier, buffer, or diluent. In various aspects, the nucleic acid sequences, nucleic acid constructs, or vectors of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the nucleic acid sequences, nucleic acid constructs, or vectors described herein and can also include a carrier such as a pharmaceutically acceptable carrier. For example, disclosed are pharmaceutical compositions, comprising any one or more of the nucleic acid sequences, nucleic acid constructs, or vectors disclosed herein, and a pharmaceutically acceptable carrier. In one aspect, disclosed are pharmaceutical compositions comprising any one or more of the nucleic acid sequences, nucleic acid constructs, or vectors. That is, a pharmaceutical composition can be provided comprising a therapeutically effective amount of at least one of the nucleic acid sequences, nucleic acid constructs, or vectors or at least one product of a disclosed method and a pharmaceutically acceptable carrier.

In certain aspects, the disclosed pharmaceutical compositions comprise any one or more of the nucleic acid sequences, nucleic acid constructs, or vectors (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, any one or more of the nucleic acid sequences, nucleic acid constructs, or vectors described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration. e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The nucleic acid sequences, nucleic acid constructs, or vectors described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed nucleic acid sequences, nucleic acid constructs, or vectors in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the nucleic acid sequences, nucleic acid constructs, or vectors of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise any one or more of the nucleic acid sequences, nucleic acid constructs, or vectors disclosed herein(or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage: thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed nucleic acid sequences, nucleic acid constructs, or vectors, and/or pharmaceutically acceptable salts thereof, can also be prepared in powder or liquid concentrate form.

The exact dosage and frequency of administration depends on the particular disclosed nucleic acid sequences, nucleic acid constructs, or vectors, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### F. Methods

SMYD1 is a myocyte-specific epigenetic regulator that functions as a histone methyltransferase for control of gene expression and regulates the skeletal and cardiac muscle differentiation. In some aspects, normal levels of SMYD1 or increased levels of SMYD1 can be used to maintain a healthy heart and mitochondria.

In some aspects, SMYD1 positively regulates mitochondrial respiration and ATP production.

In some aspects, SMYD1 also regulates OPA1 expression. In some aspects, OPA1 is involved in increased cristae density, narrowing cristae junctions, increased supercomplex formation, enhanced respiration and protection from ischemic injury. Thus, in some aspects, increasing SMYD1 in a subject (e.g., the cell (cardiomyocyte) of a subject) can result in positive effects of the OPA1 pathway.

Disclosed herein are methods of using SMYD1 to provide positive effects on the heart or any muscle cell, including protecting the heart from ischemic injury.

In some aspects, the disclosed methods can be used to increase Smyd1 expression in a cell in a subject by administering to a subject or to a cell in a subject, one or more of the nucleic acid sequences, nucleic acid constructs, or vectors described herein.

In some aspects, the disclosed methods are gene therapy based wherein a nucleic acid sequence encoding SMYD1 can be administered to a subject or to a sample comprising cells or tissue.

### 1. Methods of Altering Biological Functions Related to SMYD1

Disclosed are methods of reducing infarct size in a subject comprising increasing SMYD1 in the subject. In some aspects, the infarct can have occurred due to anything that caused a failure of blood supply to tissue. In some aspects, infarcts can occur due to the presence of a blood clot. In some aspects, reducing the infarct size can be reducing the infarct size by 1, 2, 3, 4, 5, 6, 7, 8, 9, 10, 15, 20, 25, 30, 35, 40, 45, 50, 55, 60, 65, 70, 75, 80, 85, 90, 95, 99, 100% compared to before increasing SMYD1. In some aspects, a reduction in infarct size can be determined by an increase (or no further decrease) in the ejection fraction of the left ventricle. In some aspects an increase in ejection fraction can be a 1, 2, 3, 4, 5, 6, 7, 8, 9, 10, 15, 20, 25, 30, 35, 40, 45, 50, 55, 60, 65, 70, 75, 80, 85, 90, 95, 99, 100% increase compared to before increasing SMYD1.

Disclosed are methods of reducing myocyte death in a subject comprising increasing SMYD1 in the subject. In some aspects, increasing SMYD1 in the subject can be achieved by increasing Smyd1 expression in a cell in a subject by administering to a subject or to a cell in a subject, one or more of the nucleic acid sequences, nucleic acid constructs, or vectors described herein. In some aspects, ischemic injury can cause myocyte death. In some aspects, myocyte death caused by ischemic injury can be reduced by increasing SMYD1 in a subject having undergone ischemic injury.

Disclosed are method of enhancing mitochondrial respiration in a cell comprising increasing SMYD1 in the cell. In some aspects, increasing SMYD1 in the subject can be achieved by increasing Smyd1 expression in a cell in a subject by administering to a subject or to a cell in a subject, one or more of the nucleic acid sequences, nucleic acid constructs, or vectors described herein

Disclosed are methods of increasing ATP production in a cell comprising increasing SMYD1 in the cell. In some aspects, increasing SMYD1 in the subject can be achieved by increasing Smyd1 expression in a cell in a subject by administering to a subject or to a cell in a subject, one or more of the nucleic acid sequences, nucleic acid constructs, or vectors described herein. In some aspects, deficits in ATP production have been linked to the development of heart failure and ischemic injury. Thus, increasing SMYD1 can both increase ATP production and ultimately protect against heart failure or ischemic injury.

In some aspects, increasing SMYD1 in any of the disclosed methods includes, but is not limited to, administering a nucleic acid that encodes SMDY1a, administering a SMYD1 protein, administering a compound or molecule that regulates expression of SMYD1, administering a compound or molecule that prevents degradation of SMYD1 or stabilizes SMYD1 mRNA to the subject or to a cell in the subject.

In some aspects, increasing SMYD1 comprises increasing Smyd1 expression. In some aspects, increasing Smyd1 expression can include increasing expression of the natural Smyd1 gene and/or increasing expression of an exogenous Smyd1 gene. In some aspects, increasing Smyd1 expression comprises administering a nucleic acid sequence that encodes SMDY1a or encodes a protein that regulates expression of Smyd1 or administering a compound or molecule that regulates expression of Smyd1. In some aspects, increasing SMYD1 in the subject comprises administering a therapeutically effective amount of a composition that increases Smyd1 expression. In some aspects, the composition can comprise a nucleic acid sequence that encodes SMYD1 or encodes a protein that regulates SMYD1 expression. In some aspects, increasing Smyd1 expression in the subject comprises administering a therapeutically effective amount of a vector comprising a nucleic acid sequence that encodes SMYD1 or a protein that regulates smyd1 expression to the subject. In some aspects, a vector can be any of those described herein. For example, the vector can be a viral vector or non-viral vector. In some aspects, a vector can be nucleic acid based or non-nucleic acid based. In some aspects, the viral vector is an adeno-associated viral (AAV) vector. In some aspects, the AAV vector can be any one of AAV1-9. In some aspects, the non-viral vector can be a nucleic acid based vector (e.g., plasmid), liposomes and lipid nanoparticles (LNPs), highly branched poly(β-amino ester) (HPAE), single-chain cyclic polymer (SCKP), poly(amidoamine) (PAMAM) dendrimers, or polyethyleneimine (PEI). In some aspects, the nucleic acid based vector can comprise a promoter, such as an inducible promoter.

In some aspects, increasing SMYD1 in the subject comprises administering a therapeutically effective amount of SMYD1 to the subject. In some aspects, increasing SMYD1 is different than increasing smyd1 expression in that SMYD1 can be increased by administering a SMYD1 protein to a subject without having to alter expression of smyd1 gene in the subject.

In some aspects, increasing SMYD1 in the subject comprises administering a therapeutically effective amount of a compound or molecule (e.g., protein, nucleic acid) that controls or regulates the degradation of SMDY1a in the subject. For example, a compound or molecule that prevents or reduces SMYD1 degradation indirectly increases SMYD1 in a subject. In some aspects, a compound or molecule stabilizes smyd1 mRNA.

In some aspects, as used throughout, methods of increasing SMYD1 in the subject can include maintaining natural levels of SMYD1 in the subject. For example, in some aspects a medical condition can cause a decrease in SMYD1, thus, administering a composition that maintains natural levels of SMYD1, thus preventing the decrease of SMYD1, can be considered a method of increasing SMYD1.

2. Methods of Protecting a Heart from Ischemic Injury

Disclosed are methods of protecting a heart from ischemic injury comprising increasing SMYD1 in the heart. Also disclosed are methods of protecting a heart from any heart condition such as, but not limited to, diabetic cardiomyopathy, hypertrophic cardiomyopathy, or non-ischemic cardiomyopathy.

In some aspects, increasing SMYD1 in any of the disclosed methods includes, but is not limited to, administering a nucleic acid that encodes SMYD1, administering a SMYD1 protein, administering a compound or molecule that regulates expression of smyd1, administering a compound or molecule that prevents degradation of SMYD1 or stabilizes smyd1 mRNA.

In some aspects, increasing SMYD1 comprises increasing smyd1 expression. In some aspects, increasing SMYD1 expression can include increasing expression of the natural SMYD1 gene and/or increasing expression of an exogenous SMYD1 gene. In some aspects, increasing SMYD1 expression comprises administering a nucleic acid sequence that encodes SMDY1a or encodes a protein that regulates expression of SMYD1 or administering a compound or molecule that regulates expression of smyd1. In some aspects, increasing SMYD1 in the subject comprises administering a therapeutically effective amount of a composition that increases smyd1 expression. In some aspects, the composition can comprise a nucleic acid sequence that encodes SMYD1 or encodes a protein that regulates smyd1 expression. In some aspects, increasing smyd1 expression in the subject comprises administering a therapeutically effective amount of a vector comprising a nucleic acid sequence that encodes SMYD1 or a protein that regulates smyd1 expression to the subject. In some aspects, a vector can be any of those described herein. For example, the vector can be a viral vector or non-viral vector. In some aspects, a vector can be nucleic acid based or non-nucleic acid based. In some aspects, the viral vector is an adeno-associated viral (AAV) vector. In some aspects, the AAV vector can be any one of AAV1-9. In some aspects, the non-viral vector can be a nucleic acid based vector (e.g., plasmid), liposomes and lipid nanoparticles (LNPs), highly branched poly(β-amino ester) (HPAE), single-chain cyclic polymer (SCKP), poly(amidoamine) (PAMAM) dendrimers, or polyethyleneimine (PEI). In some aspects, the a nucleic acid based vector can comprise a promoter, such as an inducible promoter.

In some aspects, increasing SMYD1 in the subject comprises administering a therapeutically effective amount of SMYD1 to the subject. In some aspects, increasing SMYD1 is different than increasing smyd1 expression in that SMYD1 can be increased by administering a SMYD1 protein to a subject without having to alter expression of smyd1 in the subject.

In some aspects, increasing SMYD1 in the subject comprises administering a therapeutically effective amount of a compound or molecule (e.g., protein, nucleic acid) that controls or regulates the degradation of SMDY1a in the subject. For example, a compound or molecule that prevents or reduces SMYD1 degradation indirectly increases SMYD1 in a subject. In some aspects, a compound or molecule stabilizes smyd1 mRNA.

In some aspects, as used throughout, methods of increasing SMYD1 in the subject can include maintaining natural levels of SMYD1 in the subject. For example, in some aspects a medical condition can cause a decrease in SMYD1, thus, administering a composition that maintains natural levels of SMYD1, thus preventing the decrease of SMYD1, can be considered a method of increasing SMYD1.

### 3. Methods of Treating

Disclosed are methods of treating a subject having ischemic heart disease or a cardiomyopathy or at risk of ischemic heart disease or a cardiomyopathy comprising increasing SMYD1 in the heart. In some aspects, cardiomyopathy is a group of diseases that affect the heart muscle and can cause the heart muscle to thicken, stiffen, thin, or fill with substances that don't belong there. Disclosed are methods of treating a subject having any heart condition such as, but not limited to, diabetic cardiomyopathy, hypertrophic cardiomyopathy, or non-ischemic cardiomyopathy.

In some aspects, increasing SMYD1 in any of the disclosed methods includes, but is not limited to, administering a nucleic acid that encodes SMYD1, administering a SMYD1 protein, administering a compound or molecule that regulates expression of smyd1, administering a compound or molecule that prevents degradation of SMYD1 or stabilizes smyd1 mRNA.

In some aspects, increasing SMYD1 comprises increasing smyd1 expression. In some aspects, increasing SMYD1 expression can include increasing expression of the natural SMYD1 gene and/or increasing expression of an exogenous SMYD1 gene. In some aspects, increasing SMYD1 expression comprises administering a nucleic acid sequence that encodes SMDY1a or encodes a protein that regulates expression of SMYD1 or administering a compound or molecule that regulates expression of smyd1. In some aspects, increasing SMYD1 in the subject comprises administering a therapeutically effective amount of a composition that increases smyd1 expression. In some aspects, the composition can comprise a nucleic acid sequence that encodes SMYD1 or encodes a protein that regulates smyd1 expression. In some aspects, increasing Smyd1 expression in the subject comprises administering a therapeutically effective amount of a vector comprising a nucleic acid sequence that encodes SMYD1 or a protein that regulates smyd1 expression to the subject. In some aspects, a vector can be any of those described herein. For example, the vector can be a viral vector or non-viral vector. In some aspects, a vector can be nucleic acid based or non-nucleic acid based. In some aspects, the viral vector is an adeno-associated viral (AAV) vector. In some aspects, the AAV vector can be any one of AAV1-9. In some aspects, the non-viral vector can be a nucleic acid based vector (e.g., plasmid), liposomes and lipid nanoparticles (LNPs), highly branched poly(β-amino ester) (HPAE), single-chain cyclic polymer (SCKP), poly(amidoamine) (PAMAM) dendrimers, or polyethyleneimine (PEI). In some aspects, the a nucleic acid based vector can comprise a promoter, such as an inducible promoter.

In some aspects, increasing SMYD1 in the subject comprises administering a therapeutically effective amount of SMYD1 to the subject. In some aspects, increasing SMYD1 is different than increasing smyd1 expression in that SMYD1 can be increased by administering a SMYD1 protein to a subject without having to alter expression of smyd1 in the subject.

In some aspects, increasing SMYD1 in the subject comprises administering a therapeutically effective amount of a compound or molecule (e.g., protein, nucleic acid) that controls or regulates the degradation of SMDY1a in the subject. For example, a compound or molecule that prevents or reduces SMYD1 degradation indirectly increases SMYD1 in a subject. In some aspects, a compound or molecule stabilizes smyd1 mRNA.

In some aspects, as used throughout, methods of increasing SMYD1 in the subject can include maintaining natural levels of SMYD1 in the subject. For example, in some aspects a medical condition can cause a decrease in SMYD1, thus, administering a composition that maintains natural levels of SMYD1, thus preventing the decrease of SMYD1, can be considered a method of increasing SMYD1.

In some aspects, increasing SMYD1 occurs within 30 minutes, 1 hour, 2 hours. 3 hours, 4 hours, 5 hours, 6 hours, 7 hours, 8 hours, 9 hours, 10 hours, 12, hours, 24 hours, 48 hours of an ischemic injury or a cardiomyopathy. In some aspects, increasing SMYD1 occurs 30 minutes, 1 hour, 2 hours, 3 hours, 4 hours, 5 hours, 6 hours, 7 hours, 8 hours, 9 hours, 10 hours, 12, hours, 24 hours, 48 hours prior to an ischemic injury or a cardiomyopathy.

### G. Combination Therapy

In one aspect of the disclosed methods, the compositions can be administered alone or in combination with one or more additional therapeutic agents. The additional therapeutic agents are selected based on the condition to be treated. A description of the various classes of suitable pharmacological agents and drugs may be found in Goodman and Gilman, The Pharmacological Basis of Therapeutics, (11th Ed., McGraw-Hill Publishing Co.) (2005).

In some aspects, any known treatments for ischemic injury can be administered with the disclosed compositions for increasing SMYD1. For example, disclosed are methods of protecting a heart from ischemic injury comprising increasing SMYD1 in the heart by administering a therapeutically effective amount of a vector comprising a nucleic acid sequence that encodes SMYD1 or a protein that regulates SMYD1 expression to the subject and administering a therapeutically effective amount of Alteplase (i.e., tissue plasminogen activator) to the subject.

### H. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed compositions, vectors, nucleic acid constructs, nucleic acid sequences.

### Examples

### A. Introduction

Described herein is a newly generated mouse model capable of inducible, cardiomyocyte-specific overexpression of SMYD1 in transgenic mice (TG) and demonstrate for the first time that SMYD1 gain-of-function can positively regulate mitochondrial respiration in an animal model. This enhanced respiration protects the heart from ischemic injury and reduces infarct size by >50%. These mice are also capable of maintaining basal levels of PGC-1α expression and its downstream targets, including electron transport chain subunits, after ischemic injury. In addition, the data from SMYD1 overexpressing mice show increased mitochondrial cristae formation and stabilization of respiratory chain supercomplexes within the cristae, concomitant with increased OPA1 expression, a known driver of cristae morphology. Together these results demonstrate that SMYD1 protects from ischemic injury by regulating mitochondrial energetics and enhancing respiration efficiency in the cardiomyocyte through the synergistic regulation of both PGC-1α and OPA1. This work highlights that SMYD1 is the only known epigenetic regulator of cristae morphology. It provides broad implications for understanding the epigenetic mechanisms driving cardiac energetics and identifies a novel signaling pathway by which cardiomyocytes regulate energy efficiency, protecting them from ischemic injury.

### B. Materials and Methods.

1. Generation of Transgenic Mice which Overexpress SMYD1.

To achieve cardiomyocyte-specific overexpression of mouse ortholog of human SMYD1, which is histone lysine N-methyltransferase Smyd1 isoform 1 (GenBank: NM_001160127.1), referred to as SMYD1, animals expressing a reverse tetracycline-controlled transactivator (rtTA) under the aMHC promoter were crossed with mice carrying the SMYD1 gene (tagged with FLAG on the C-terminus) under a modified aMHC promoter containing a TRE (7 tet-o repeats) element. Resulting animals were SMYD1^{TRE+}/rtTA^{+/-}, referred to as transgenic (TG) or SMYD1-OE, and SMYD1^{TRE-/-}/rtTA^{+/-} referred to as WT littermate controls. Animals generated were maintained on a FVB background, housed under standard conditions, and given food and water ad libitum. Primers used for genotyping are listed in Table 3.

TABLE 3
List of primers used in this study.

| | | |
|---|---|---|
| β-Actin-mouse | Forward | TGT TAC CAA CTG GGA CGA |
| | Reverse | GGG GTG TTG AAG GTC TCA |
| Nppa (ANF)-mouse | Forward | CTG ATG GAT TTC AAG AAC CTG CT |
| | Reverse | CTC TGG GCT CCA ATC CTG TC |
| Myh6 (αMHC)-mouse | Forward | GAA CAG CTG GGA GAA GGG GG |
| | Reverse | GCC TCT GAG GCT ATT CIA TTG G |
| Myh7 (αMHC)-mouse | Forward | GAA CAG CTG GGA GAA GGG GG |
| | Reverse | GCC TCT GAG GCT ATT CTA TTG G |
| Apt2a2 (Serca2a)- | Forward | CCT TCT ACC AGC TGA GTC ATT T |
| mouse | Reverse | CAG ATG GAG CCC ACG ACC CA |
| Ppargc1 (PGC- | Forward | CTT CGA GCT GTA CTT TTG TGG ACG GAA |
| 1α)-mouse | Reverse | CTC TGA GCT TCC TTC AGT AAA CTA TCA AA |
| Vim-mouse | Forward | TGA GAT CGC CAC CTA CAG GA |
| | Reverse | TGA TCA CCT GTC CAT CTC TGG |
| Col1a1-mouse | Forward | GGT CAG ACC TGT GTG TTC CC |
| | Reverse | GGT CCA TGT AGG CTA CGC TG |
| SMYD1- mouse | Forward | GTG AAA TCC ATG TTT CAC ACG CAG ATG AG |
| | Reverse | CAG GAA GAG GTC GTC CTT CAG C |
| Smyd1b-mouse | Forward | CTG CAC TGT CAT ATT CAA CAA TGG CAA GAT |
| | Reverse | CAG GAA GAG GTC GTC CTT CAG C |
| Smyd1-human | Forward | GCC CAT TAC TGC GAC CGC AC |
| | Reverse | CTC CCC AAA GTG CTC CAC GTG G |
| Smyd1-mouse | Forward | CTC GCT CCG AGG GTT TGT ATC ACG |
| genotyping | Reverse | CTT ATC GTC GTC ATC CTT GTA ATC CAG G |
| Smyd2-mouse | Forward | GGA GGG CCA AAC ACT ACA AA |
| | Reverse | TGA GGG AGT ACA CGG GGT AG |
| Smyd3-mouse | Forward | TGA TGA AAG TTG GCA AGC TG |
| | Reverse | GTC CTT CTG GGG GTC CTT G |
| Smyd4-mouse | Forward | CGA ACC GGT AAA CCA GAG CA |

To induce SMYD1 overexpression mice were fed a special chow diet containing 625 mg/kg of doxycycline hyclate (TD. 130141 from Envigo) at a daily dose of 2-3 mg for the length of study. DOX-induced expression of SMYD1 in the heart was confirmed by qPCR, western blotting and LC-MS/MS in at least three separate experiments.

### 2. Smyd1-Knockout Mice

Inducible, cardiomyocyte-specific Smyd1-knockout mice (Smyd1^{flox/flox} Cre^{+/-}) and control mice (Smyd1^{flox/flox} Cre^{-/-}) were developed as previously described [20, 61]. Mice at 8 weeks of age were fed tamoxifen containing chow (0.4 mg/g of chow diet, TD.07262, Harlan) for 3 weeks at which time their hearts were harvested, ventricles separated and immediately frozen in liquid nitrogen and stored at −80° C. until they were used for analysis.

### 3. Surgeries

Permanent occlusion (PO) of the left anterior descending artery (LAD) or Sham surgeries were performed 2 weeks after DOX-induced overexpression of SMYD1 in adult (16-19 week) SMYD1-transgenic (TG) mice and their littermate controls (WT), with continued doxycycline chow administration. Surgeries were performed using aseptic technique in a dedicated surgery facility. Animals were anesthetized with 2% isoflurane. In the supine position, endotracheal intubation was performed, and mice were ventilated with a small animal respirator. After the pericardiectomy, the left anterior descending (LAD) coronary artery was visualized under the microscope. A suture was placed through the myocardium underneath and around the LAD, approximately midway between the apex and the base, and tied with a surgeon's knot. The apex of the left ventricle was observed for evidence of myocardial blanching indicating interruption in coronary flow. The Sham (control) procedure was identical except that the LAD was not ligated. The mice were randomly assigned to a surgeon who was blinded to the genotypes during the surgical procedures. Cardiac function was monitored prior to and once a week following PO surgeries as detailed below by echocardiography. Mice were ultimately euthanized at 24 hours, 48 hours, or 5-weeks post-operatively to obtain cardiac tissue for histological and biochemical analyses.

### 4. Mouse Cardiac Tissue Collection

Cardiac tissue was harvested from mice and rinsed with phosphate-buffered saline (PBS), drained and weighed, followed by atrial and vascular tissue removal to leave the ventricles. Heart weight (HW), body weight (BW) and tibia length (TL) were measured to calculate HW/BW and HW/TL ratios for evaluation of hypertrophic response to PO. Extracted heart ventricles were cut in to four 1.5 mm sections using a mouse heart slicer matrix (Zivic Instruments). Tissue sections, except for histology, were immediately frozen in liquid nitrogen and stored at −80° C. until use.

### 5. Human Cardiac Tissue

Patients (age≥18-years) were prospectively enrolled in institutions with clinical characteristics consistent with dilated cardiomyopathy and chronic advanced heart failure who required circulatory support with continuous flow Left Ventricular Assist Device (LVAD) as a bridge to transplantation or lifetime destination therapy. Patients who required LVAD support due to acute heart failure (acute myocardial infarction, acute myocarditis, post-cardiotomy cardiogenic shock, etc.) were prospectively excluded. LVAD patients underwent serial echocardiograms monthly for the first three months and at 4.5 and 6 months. They were categorized as either responders or non-responders using left ventricular ejection fraction (LVEF) and left ventricular end-diastolic diameter (LVEDD) measurements during diminished LVAD support “turn-down” echocardiograms). Responders were defined as patients with a LVEF>40% and LVEDD≤5.9 cm at six months post LVAD implantation, whereas non-responders were defined as patients with a LVEF <35% and with <50% relative improvement in LVEF regardless of the final LVEDD. The study was approved by the institutional review board of the participating institutions, and informed consent was provided by all patients. For heart failure patients, their clinical demographics, echocardiographic parameters, protein biomarkers and other clinical data were prospectively collected and entered in the programs research electronic data capture system (REDCap). Myocardial tissue from donor hearts, not allocated for heart transplantation due to non-cardiac reasons (size, infection, etc.), were used as non-failing controls. Myocardial tissue was prospectively collected from the LV apical core at the time of LVAD implantation and was snap frozen before storing it at −80° C. as described before. Donor control samples were acquired from hearts that were not transplanted due to non-cardiac reasons and LV apical tissue was harvested and processed the same way as the failing hearts. Clinical characteristics of the study population from subjects with advance heart failure grouped as responders and non-responders as well as from donors' group is presented in Table 1 and 2, respectively.

TABLE 1
Clinical characteristics of the study population. Left ventricular
ejection fraction (LVEF) and left ventricular end diastolic
diameter (LVEDD) at both LVAD implantation (pre-LVAD) and
LVAD explantation/cardiac transplantation (post-LVAD) showing
clinical differences between Responders and Non-Responders.
(Values reported as Mean ± SEM).

| | Responders | | Non-responders | | p- |
|---|---|---|---|---|---|
| Variable | N = 5 | | N = 5 | | value |
| Male sex, (%) | 4 | (80) | 4 | (80) | |
| Age at LVAD | 56.2 ± 9.7 | | 54.2 ± 7.6 | | 0.876 |
| implantation, years | | | | | |
| Pre-LVAD | | | | | |
| LVEDD, cm | 5.2 ± 0.5 | | 6.2 ± 0.5 | | 0.159 |
| LVEF, % | 38.8 ± 7.2 | | 16.2 ± 3.1 | | 0.017 |
| Post-LVAD | | | | | |
| LVEDD, cm | 5.0 ± 0.2 | | 5.8 ± 0.6 | | 0.277 |
| LVEF, % | 45.0 ± 3.9 | | 23.6 ± 2.6 | | 0.002 |
| HF Etiology | | | | | |
| Ischemic | 2 | (40.0) | 2 | (40.0) | |
| cardiomyopathy, n (%) | | | | | |
| Non-ischemic | 3 | (60.0) | 3 | (60.0) | |
| cardiomyopathy, n (%) | | | | | |
| New York Heart Association | | | | | |
| Functional Class | | | | | |
| III, n (%) | 2 | (40.0) | 2 | (40.0) | |
| IV, n (%) | 3 | (60.0) | 3 | (60.0) | |

TABLE 2
Clinical characteristics of the study population, donors.

| | Donors, N = 5, |
|---|---|
| Variable | (Mean ± SEM) |
| Female, n (%) | 5 (100%) |
| Age | 38 ± 12 |
| Weight | 72 ± 16 |
| Cause of death | |
| CVA Stroke | 40% |
| Anoxia | 40% |
| Head Trauma | 20% |

CVA—cardiovascular accident.

### 6. Echocardiography

In vivo cardiac function of mice was performed by echocardiography, using a Vevo 2100 ultrasound machine. The following indices were examined: left ventricular size (end diastolic and systolic dimensions), wall thickness, ventricular mass, and ventricular function (ejection fraction and fractional shortening).

### 7. Histological Analysis

Heart tissue from transgenic (TG) mice and their littermate controls (WT) were harvested for histology analyses and evaluation for gross morphology and cellular level parameters. Briefly, hearts were fixed using 4% paraformaldehyde and submitted to the Biorepository and Molecular Pathology Shared Resource at Huntsman Cancer Institute at the University of Utah, for paraffin embedding and sectioning (4 μm). Tissue sections were stained with Masson's trichrome and Hematoxylin and Eosin (H&E). Sections were visualized using an Olympus BX51WI microscope with a DP73 color camera and image analysis was performed with CellSens software (Olympus). To quantify the area of infarct: Trichrome and H&E slides were evaluated by an ACVP-board certified veterinary pathologist. One MT image per heart (n=5-9/group was imported to ImageJ software for measurement of midline infarct length. The infarct size was calculated as infarct length/left ventricular circumference*100. Samples with lesions affecting <50% of the thickness of the ventricle were recorded as “0” for the infarct size. H&E-stained slides (n=3-4/group) containing multiple sections of heart per animal were also scored according to the extent of lesioned area: 0=absent, 1=minimal (<10% affected). 2=mild (10-25% affected), 3=moderate (26-50% affected). 4=marked (51-75% affected). 5=severe (>75% affected). The scores for each section were summed for individual animals.

### 8. High-Resolution Respirometry

To induce cardiac-specific overexpression of SMYD1, the transgenic mice were maintained on doxycycline chow for 2 weeks before harvest. Mitochondrial O₂ utilization was measured using the Oroboros O2K Oxygraphs, as previously described [29]. Cardiac tissue was minced in mitochondrial isolation medium (300 mM sucrose, 10 mM HEPES, 1 mM EGTA at pH 7.2) and subsequently homogenized using a Teflon-glass system. Homogenates were then centrifuged at 800×g, 4° C. for 10 min, after which the supernatant was separated and centrifuged at 12,000×g, 4° C. for 10 min. The resulting pellet was resuspended in mitochondrial isolation medium. Isolated mitochondria in the amount of 12.5 mg/mL were then added to the Oxygraph chambers containing assay buffers (105 mM MES potassium salt, 30 mM KCl, 10 mM KH₂PO₄, 5 mM MgCL₂, 0.5 mg/mL BSA). Complex I/II supported respiration and maximal respiration was measured in response to the following substrates added in sequence: 0.5 mM malate, 5 mM pyruvate, 2.5 mM ADP, 5 mM glutamate, 10 mM succinate, 1.5 mM FCCP. Fatty Acid-supported oxidation was measured in response to the following substrates: 0.02 mM palmitoyl-carnitine, 5 mM L-carnitine, 2.5 mM ADP. Data were processed using Oroboros DatLab7 software and statistical analysis was performed using Excel.

### 9. ATP Production

Mitochondria were isolated as described above. To quantify ATP production per O₂ reduction (ATP:O ratio) high-resolution respirometry (Oroboros O2k oxygraphs) was used, coupled with fluorometry (Horiba Fluoromax-4). Briefly, isolated mitochondria in the amount of 2.5 mg/mL were added to the fluorometer chamber containing assay buffers (105 mM MES potassium salt, 30 mM KCl, 10 mM KH₂PO₄, 5 mM MgCl₂, 0.5 mg/mL BSA, 1 U/mL Hexokinase, 2.5 U/mL Glucose-6-Phosphate Dehydrogenase. 2.5 mM D-glucose, 2.5 mM NADP+, 200 μM P1,P5-di(adenosine-5′) pentaphosphate). ATP production was measured in response to the following substrates: 0.5 mM malate, 5 mM pyruvate, 2.5 mM ADP. Data were processed using Oroboros DatLab7 software and Excel and statistical analysis was performed using Excel.

### 10. Cell Mito Stress Test

To evaluate mitochondrial function, the Cell Mito Stress Test was performed in H9c2 cells using a Seahorse XFe96 Flux Analyzer (Agilent). Briefly, the H9c2 cells were plated in 96-well Seahorse analyzer plates pre-coated with laminin (20 k cells per well) and transduced with adenovirus carrying SMYD1-FLAG at a multiplicity of infection (MOI) of 100. Cells not treated with adenovirus were used as controls. After 48 h, oxygen consumption rates (OCR) were measured in XF base medium (Agilent-Seahorse) at pH 7.4 and supplemented with 2 mM L-glutamine, 1 mM pyruvate and 25 mM glucose. The OCR rates were measured before and after sequential addition of inhibitors: oligomycin (1 μM), carbonyl cyanide 4-(trifluoromethoxy) phenylhydrazone (FCCP, 5 μM) and mix of rotenone (1 μM) and antimycin A (1 μM). The OCR values were normalized to the intensity of nuclear staining. For the palmitate oxidation assay, 24 h hours after adenovirus transductions, H9c2 cells were incubated in substrate-limited media containing DMEM formulated without glucose, glutamine, sodium pyruvate and HEPES, and supplemented with 0.5 mM glucose, 1 mM GlutaMAX, 0.5 mM L-carnitine, and 1% FBS for 24 h prior to the assay, followed by incubation with either palmitate (160 M) coupled to BSA or BSA alone in Krebs-Hanseleit buffer at pH 7.4 containing 5 mM L-carnitine and 5 mM HEPES. The OCR rates in response to inhibitors were measured as described above.

### 11. TUNEL Assay

TUNEL assay was performed on cardiac tissue or cultured H9c2 cardiomyoblasts using In Situ Cell Death Detection Kit, Fluorescein, Sigma Aldrich according to the manufacturer's instructions. Briefly, paraffin-embedded tissues were cut into 5 μm-thick sections, dewaxed, rehydrated according to standard protocols. H9c2 cardiomyoblasts were plated on laminin-treated cover slips and after 24 h transduced with Ad-SMYD1 or empty virus (control). After 48 h cells were subjected to hypoxia after which they were fixed and permeabilized according to standard protocols. Samples were treated with Proteinase K, then labeled using TUNEL reaction mix. Tissue sections were stained for cardiomyocytes using standard immunohistochemistry methods with cardiac troponin C antibody (Abcam, ab137130). Then samples were washed with IX PBS before mounting on coverslips using an aqueous-based fluorescence mounting medium containing DAPI (Prolong Gold). Negative controls were used to exclude false positives by incubating fixed and permeabilized sections in label solution without terminal transferase instead of TUNEL reaction mixture. Positive controls were generated by incubating fixed and permeabilized sections with recombinant DNase I to induce DNA strand breaks. Images were acquired using a fluorescence microscope with an excitation wavelength in the range of 450-500 nm and detection in the range of 515-565 nm (green). The TUNEL-positive rate was calculated and expressed as the number of TUNEL-positive cells divided by the total number of nuclei counted.

### 12. Isolated Neonatal Rat Cardiomyocytes

Neonatal Rat Ventricular Myocytes (NRVMs) were isolated from Sprague-Dawley neonatal rats using Neonatal Cardiomyocyte Isolation System kit (Worthington). Briefly, NRVMs were isolated by enzymatic digestion from 0-1 day old litters and plated at 50% confluency in growth medium (DMEM supplemented with 10% FBS media, 1% penicillin-streptomycin (P/S), L-glutamine, D-glucose) for 24 hours and maintained in serum-free media thereafter (DMEM supplemented with 1% insulin-transferrin-selenium (ITS), 1% P/S, L-glutamine, D-glucose). Overexpression of SMYD1 was induced using adenovirus carrying FLAG tagged SMYD1 or an empty virus as a control at a MOI of 25.

### 13. Cultured H9c2 Cardiomyoblasts and Imaging

H9c2 cardiomyoblasts were cultured at 70% confluency in growth medium (DMEM supplemented with 10% FBS, 1% penicillin-streptomycin (P/S). Smyd1 or Opal knockdown was induced using respective siRNAs (Qiagen) or scrambled-siRNA (Qiagen) as a control, and lipofectamine RNAiMAX reagent (Thermo Fisher Scientific) in OPTI-MEM (Gibco). For the overexpression study, H9c2 cardiomyoblasts were transduced with adenovirus expressing SMYD1-FLAG (Ad-SMDYD1a) or empty virus (Control) at a multiplicity of infection (MOI) of 150. All cells were cultured for 48 h or 72 h before harvest for end point experiments. For ROS and calcium imaging, cells were cultured in normoxia for 48 h after transduction with adenovirus, followed by 4 h incubation in hypoxic condition, as described below. Approximately 30 min before the end of 4 h-incubation time cells were loaded with 5 M MitoSox (Thermo Fisher Scientific), or 5 μM X-Rhod1 (Thermo Fisher Scientific) and returned to hypoxic chamber for the remainder of the time. Immediately after this time cells were imaged with a Zeiss LSM 510 confocal microscope. For either dye measurements were performed with an excitation and emission at 548 nm and 574 nm. All cells were imaged at the same power and gain settings. Image analysis was performed using Cell Profiler.

### 14. Hypoxia Protocol for Cultured Cells

Cells were cultured in hypoxic conditions. Briefly, a glucose-free, serum-free “hypoxic” media was made using Dulbecco's modified eagle's medium (DMEM) without glucose, L-glutamine, phenol red, sodium pyruvate or sodium bicarbonate (Sigma, D5030). Potassium concentration of the media was doubled by adding 0.4 g/L KCl and the pH was adjusted to 6.5 to simulate typical ischemic conditions of hyperkalemia and acidosis. The “hypoxic” DMEM was gassed with 95% nitrogen, 5% carbon dioxide for 1 h prior to use to expel any excess oxygen. H9c2 cardiomyoblasts were placed in a sealed container at 37° C. with constant gas flow of 95% nitrogen, 5% carbon dioxide to ensure complete depletion of oxygen from the solution. Hypoxia lasted for 4 h, after which cells were removed from the container and immediately imaged. Control cells were kept at 37° C. for the same amount of time that the experimental cells were exposed to hypoxic conditions. Imaging took place immediately following hypoxia.

### 15. Calcium Retention Capacity (CRC)

The CRC assay was performed. Briefly, imaging was performed in 96-well plate on a Cytation 5 microplate reader. Cells (0.5 million) were incubated in 100 μL of imaging solution containing (mM): 125 KCl, 20 HEPES, 5 K₂HPO₄, 1 MgCl₂, and 10 M EGTA (pH to 7.2 with KOH, osmolality 290-300 mOsm/L) supplemented with 5 mM Tris-Succinate, 50 μg/mL digitonin and 1 μM Oregon Green 488 BAPTA calcium dye (Thermo Fisher Scientific). Excitation and emission wavelengths were 485/520 nm. Bolus injections of 5 μM CaCl₂ were injected into each well every five minutes for 2 hours. CRC was analyzed by calculating the amount of Ca²⁺ added to each well before a mitochondrial permeability transition (MPT) event was observed.

### 16. ChIP-qPCR

Chromatin was isolated from cardiac tissue from TG mice that overexpress SMYD1 or from NRVMs that had been transduced with FLAG-tagged SMYD1 (MOI-25 for 48 h) for adenovirus-mediated overexpression. Cardiac tissue from WT mice or cells transduced with empty adenovirus were used as negative controls in these experiments. Chromatin immunoprecipitation was performed using the commercially available ChIP-IT High Sensitivity kit (Active Motif, 53040) according to manufacturer's instructions. Chromatin bound proteins were immunoprecipitated using Anti-H3K4me3 (Abcam, ab8580), Anti-H3K9me3 (Abcam, ab8898), and Anti-FLAG (Sigma Aldrich, F1804). Immunoprecipitated DNA was analyzed by qRT-PCR using primer sets that amplified the promoter region of Ppargc1α and an intergenic region as a negative control, qRT-PCR of each biological replicate was performed in duplicate with equal immunoprecipitated samples and input. Values were normalized to input measurements, and enrichment was calculated using ΔΔCt method in Excel.

### 17. Gene Expression Analysis

Real-time qPCR analysis was performed as described previously [20]. Briefly, total RNA was isolated using Trizol reagent (Thermo Fisher Scientific) according to manufacturer's instructions. cDNA was synthesized using Superscript III First Strand Synthesis System (Life Technologies). Reactions were performed with 500 nM primers using SsoAdvanced Universal SYBR Green Supermix (Bio-Rad) per the manufacturer's recommendations for run temperature and amplification time. Quantification of gene expression was performed on a Bio-Rad CFX Connect real-time PCR detection system. Analysis was performed using the 2^{-ΔΔCt} method, using α-Actin as a housekeeping gene. Primers were based on prior publications [20] or designed on NCBI Primer-BLAST and obtained from Thermo Fisher Scientific. A list of primers used in this study is provided in the Table 3.

18. mtDNA Quantification by qPCR

Genomic DNA for assessment of mitochondrial DNA (mtDNA) was isolated from 25 mg of cardiac tissue using commercially available kit according to manufacturer's instructions (QIAGEN. 69504). Genomic DNA was added to a mixture of SYBR Green (Bio-Rad) and primers and analyzed with a Bio-Rad CFX Connect Real-Time system. Analysis of mtDNA/nDNA ratio was calculated by following the classical ΔΔCt method used for qPCR analysis.

### 19. Electrophoresis and Western Blotting

Western blotting analysis was performed. Antibodies used in this study are as follow: Anti-Smyd1 (Santa Cruz, SC2059 and Abcam, ab32482) Anti-FLAG (Sigma Aldrich F1804), Anti-SMYD2 (Abcam, ab108217), Anti-SMYD4 (Thermo Fisher Scientific, PA5-96631), Anti-HSP90 (Cell Signaling Technology, 4874S), Anti-p53 (Abcam, ab131442), Anti-PGC-1α (Abcam, ab54481), Anti-Histone H3 (Abcam, ab1791), Anti-H3 tri methyl K4 (Abcam, ab8580), Anti-beta TUBULIN (ab6046), Anti-Actin (sc-1616), Anti-NDUFS3 (Abcam, ab196019). Anti-NDUFS2 (Abcam, ab110249), Anti-NDUFV1 (GeneTex, GTX102209), Anti-NDUFV2 (Proteintech. 15301-1-AP), Anti-NDUFA9 (Abcam, ab14713). Anti-NDUFA10 (Santa Cruz, SC-376357), Anti-total OXPHOS cocktail (Abcam, ab110413) and Anti-VDAC1 (Abcam, ab14734), Anti-OPA1 (BD Biosciences, 612606), Anti-MFN2 (Abcam, ab50843), Anti-Mitofilin (Proteintech, 10179-1-AP).

### 20. LC-MS/MS and Proteomic Analysis

Proteomic analysis was performed on ventricular tissue collected from transgenic and control mice at 1, 3, 6, 8 and 10 weeks of doxycycline diet and was analyzed using label-free quantitative LC-MS/MS. Briefly, a total of 10 μg of protein lysate was prepared for filter aided sample preparation. Samples were reduced, alkylated and digested with trypsin at a 1:40 ratio for 18 hours at 37° C. Tryptic peptides were separated by a reverse phase C-18 column using a 180 min multistep gradient and analyzed on a Orbitrap Velos Pro mass spectrometer interfaced with an Easy nlc-1000 UPLC. Spectra was generated in data dependent acquisition mode and peptides were fragmented with collision induced dissociation. The top twenty MS1 peaks were analyzed at a resolution of 30,000 in rapid scan mode. Samples were run in duplicates to ensure sample reproducibility. Raw files were then processed with MaxQuant software (v 1.6.7.0) against the Uniprot Mus musculus database. Proteins were further filtered in Perseus (v 1.6.5.0) so only proteins with a minimum percentage of 70% in total have valid values. T-tests were performed in Perseus on filtered imputated data.

### 21. Transmission Electron Microscopy (TEM) on Cardiac Tissue for Mitochondria Evaluation

Cardiac tissue samples were fixed in 2.5% glutaraldehyde, 1% paraformaldehyde, and 0.1 M sodium cacodylate buffer, overnight at 4° C., following post-fixation for 1 h in 2% Osmium Tetroxide buffered with cacodylate buffer. After rinsing in filtered nanopure water, sections were stained with enbloc stain in Uranyl Acetate for 1 h at room temperature. Following the post-staining, the tissues were dehydrated through a graded series of ethanol washes finishing with absolute acetone. Infiltrations were performed by incubating the specimens at room temperature in a gradually increasing concentration of Epoxy resin (Electron Microscopy Science, Hatfield, PA). Specimens were gradually transferred from 50% resin in acetone to 100% resin and embedded and polymerized at 60° C. for 48 h. Ultrathin sections (70 nm) were generated using diamond knife (Diatome) with Leica UC 6 (Leica Micro-systems, Vienna, Austria), and post-stained for 10 min with saturated uranyl acetate and for 5 min with Reinold stain. Sections were imaged at 120 kV with JEOL 1400 Plus and analyzed for mitochondria area and cristae morphology.

### 22. BN-PAGE

For the assessment of Complex I organization, BN-PAGE was performed. Mitochondria isolated from transgenic and control cardiac tissue, or from cultured H9c2 cardiomyoblasts, in the amount of 50 μg were solubilized in 5% digitonin and lysates were resolved on NativePAGE Novex 3-12% gels (Thermo Fisher Scientific). After electrophoresis, in-gel Complex I activity was evaluated by incubating in Complex I activity substrate containing 2 mM Tris-HCl at pH7.4, 1 mg/mL NADH and 2.5 mg/mL Nitrotetrazolium Blue chloride for 20 min. After identification of individual Complex I and Complex I-containing supercomplexes bands according to the in-gel activity, they were quantified using Image J and statistical differences were calculated from four (for cardiac tissue) or three (for cultured cells) independent measurements per gel and combined from independent electrophoresis runs.

### 23. Citrate Synthase Activity Assay

Citrate synthase (CS) activity was performed on isolated cardiac mitochondria using Citrate Synthase Activity Assay Kit (Sigma-Aldrich. MAK193), according to the manufacturer's instructions. Briefly, 25 mg of cardiac tissue was minced on ice and homogenized using a glass dounce in mitochondria isolation buffer (210 mM mannitol, 70 mM sucrose, 5 mM HEPES and 1 mM EGTA at pH 7.2). Homogenate was then centrifuged at 400×g for 7 min to remove the nuclear fraction and unbroken cells. Supernatant was subsequently centrifuged at 4,000×g for 30 min to isolate the crude mitochondrial fraction. Due to a very high CS activity of isolated mitochondria, the suspension for measurement was prepared to a final concentration of 5 mg/ml. Samples were mixed with the CS assay reaction mixes in 96-well plate. The absorbance at 412 nm was measured continuously every 6 s for 5 min at room temperature using a Biotech Citation 5 plate reader. CS activity was calculated using formula provided by the manufacturer.

### 24. Statistical Analysis

Unless specifically noted, data were collected from technical triplicates of three independent experiments and presented as the means with standard error of the mean (SEM). Statistical significance was evaluated using the unpaired two-tailed Student's t-test or two-way ANOVA for pairwise comparisons. Asterisk * that indicates p<0.05 was considered statistically significant.

### C. Results

### 1. Expression of SMYD1 in Failing Heart.

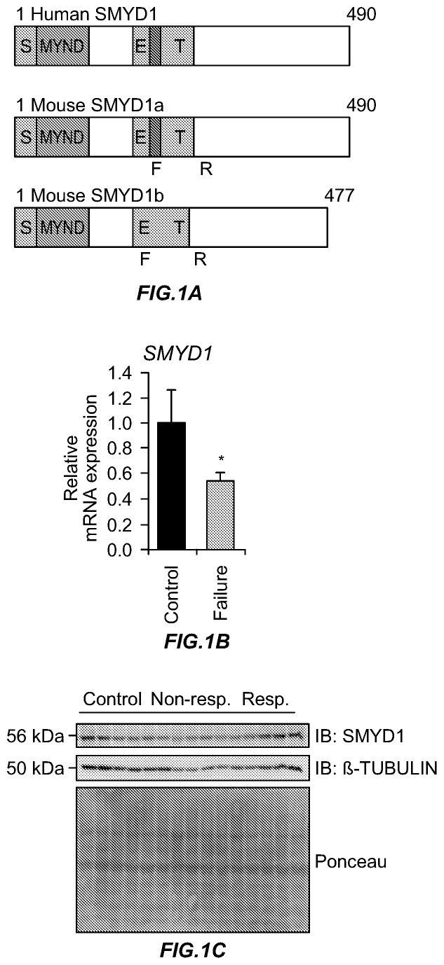

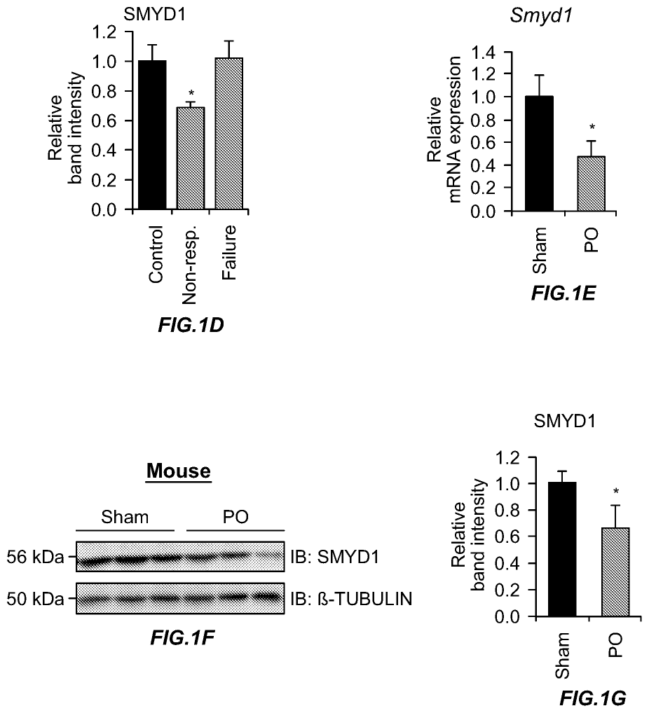

SMYD1, the founding member of the SMYD family, is a myocyte-specific lysine methyltransferase which has been shown to regulate transcription by methylating histone H3 Lysine K4. While all five members of the SMYD family are expressed in mammals, only SMYD1 is restricted to striated muscle. In humans, the SMYD1 gene produces a single transcript that shares high sequence homology and striated muscle specificity with two murine transcript variants: isoform 1 (GenBank: NM_001160127.1, referred to as SMYD1) and isoform 2 (GenBank: NM_009762.2, referred to as Smyd1b), as shown in FIG. TA. Specifically, mouse SMYD1 shares 94% sequence homology with human SMYD1, making it the mouse ortholog of human SMYD1. Knowing that SMYD1 plays a significant role in cardiac function, evaluating SMYD1 expression levels in human heart failure patients became of interest. Samples were obtained from patients with advanced heart failure who were prospectively enrolled at the time of implantation of a left ventricular assist device (LVAD) as a bridge-to-transplant or destination therapy. These samples were evaluated at the time of implantation and clinical characteristics of study subjects (heart failure) and healthy donors (control) is presented in Table 1 and 2, respectively. Interestingly. SMYD1 was significantly decreased in cardiac tissue collected from patients diagnosed with end stage heart failure at the time of LVAD implantation (FIG. 1B). This is consistent with two reports published recently, where SMYD1 transcript levels were downregulated in cardiac tissue obtained from patients with ischemic or non-ischemic cardiomyopathy undergoing LVAD implantation or from left ventricular tissue samples obtained at the time of heart transplantation. However, when these heart failure patient samples were further stratified into two categories, which described their response to LVAD unloading, the results were intriguing. Specifically, the samples were categorized as either responders or non-responders using left ventricular ejection fraction (LVEF) and left ventricular end diastolic diameter (LVEDD) measurements during diminished LVAD support “turn-down” echocardiograms. Responders were defined as patients with a LVEF >40% and LVEDD ≤5.9 cm at six months post LVAD implantation, whereas non-responders were defined as patients with a LVEF <35% and with <50% relative improvement in LVEF regardless of the final LVEDD. Protein expression analysis of these samples was performed and it was observed that while SMYD1 was significantly downregulated in patients who did not respond to LVAD therapy (non-responders), its expression was not reduced in patients whose heart function significantly improved after LVAD implantation (responders), when compared to cardiac tissue from healthy donor hearts (FIG. 1C, D). This indicates that higher levels of SMYD1 may be beneficial for the heart.

Next, SMYD1 expression was quantified in a mouse model of ischemic injury as this has never been examined before. The results showed that SMYD1 gene and protein expression in mice that were subjected to permanent occlusion (PO) of the LAD was significantly decreased 5 weeks after the surgery (FIG. 1E-G).

### 2. Generation and Validation of SMYD1 Transgenic Mice.

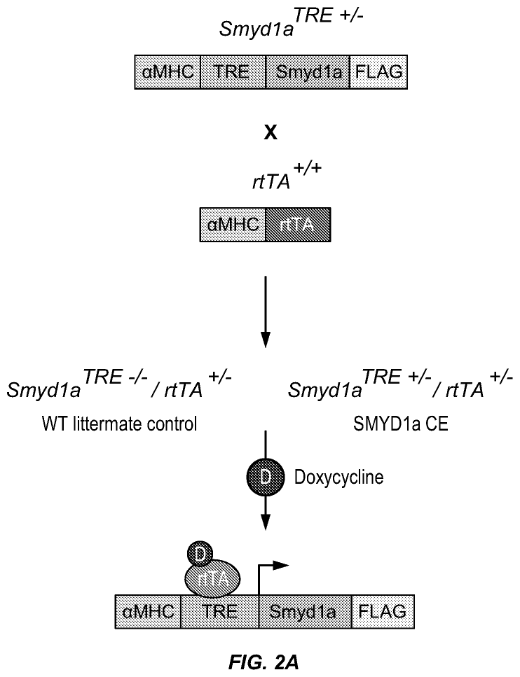

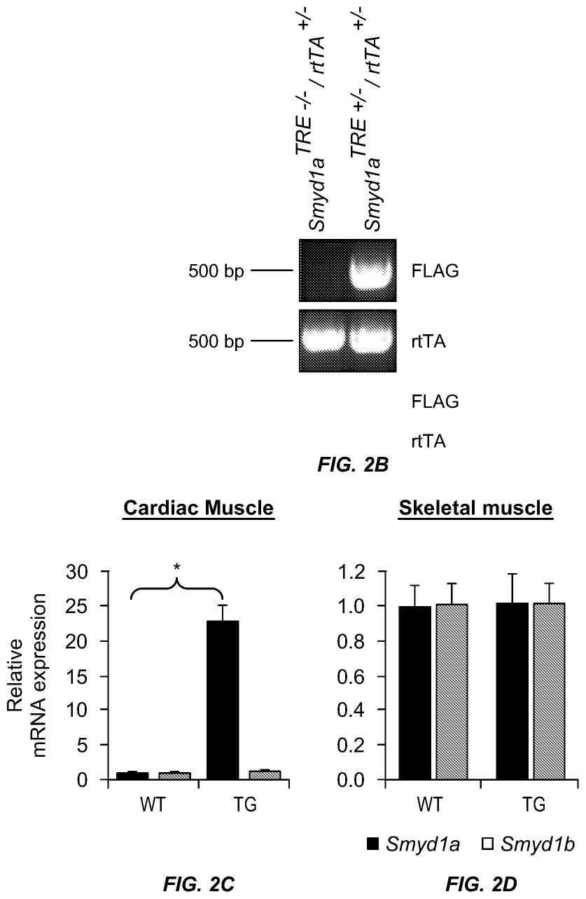

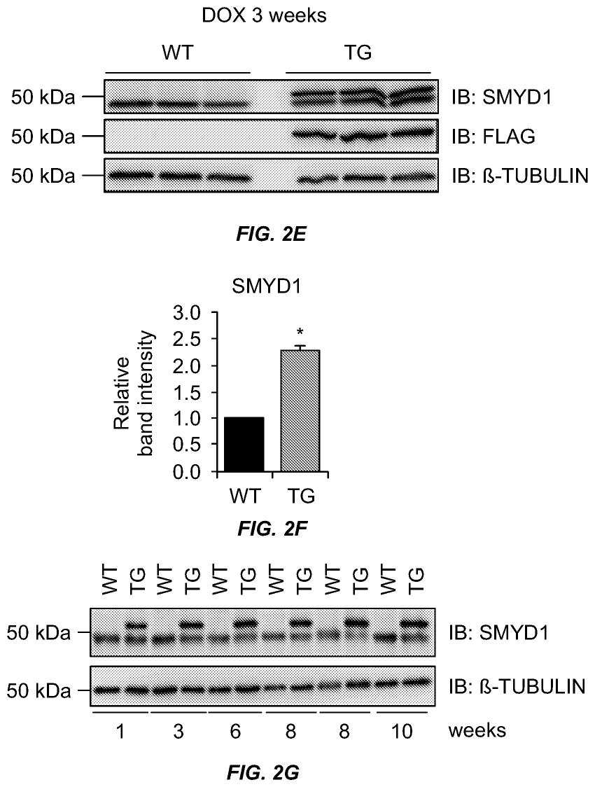

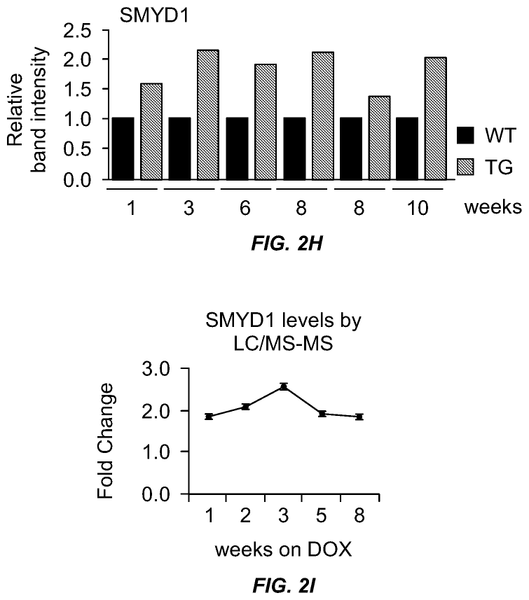

Inducible cardiomyocyte-specific knockout of Smyd1 in mice leads to pathological organ remodeling, chamber dilation and heart failure. Thus, SMYD1 gain-of-function was evaluated under basal conditions and after stress in an animal model. Transgenic mice (TG) capable of inducible, cardiomyocyte-specific overexpression of the mouse ortholog of human SMYD1 generated, which is SMYD1, were generated by crossing animals expressing a reverse tetracycline-controlled trans activator under the aMHC promoter (aMHC-rtTA) with mice carrying the SMYD1 gene (tagged with FLAG on the C-terminus) under the modified aMHC promoter containing a TRE (7 tet-o repeats) element. These mice express SMYD1-FLAG upon doxycycline (DOX) administration. The experimental scheme for generating these mice is shown in FIG. 2A, and the genotyping of these alleles is confirmed by PCR in FIG. 2B. The resulting mice were SMYD1TRE+/−/rtTA+/−, referred to as transgenic (TG) or SMYD1-OE, and SMYD1TRE−/−/rtTA+/−referred to as WT littermate controls. TG mice develop, grow and reproduce normally. These mice are fed DOX-containing chow to induce expression of SMYD1-FLAG specifically in the heart (FIG. 2C), while skeletal muscle SMYD1 remained unaffected (FIG. 2D). SMYD1 overexpression was confirmed by western blotting (FIG. 2E-H) as well as LC/MS-MS (FIG. 2I) and found that long-term DOX treatment of these mice allows for consistent ~2-fold overexpression of SMYD1. It is important to note that SMYD1-FLAG appears on western blots as a slightly higher molecular weight band than endogenous SMYD1.

3. SMYD1 Overexpression in Mice Protects from Ischemic Injury.

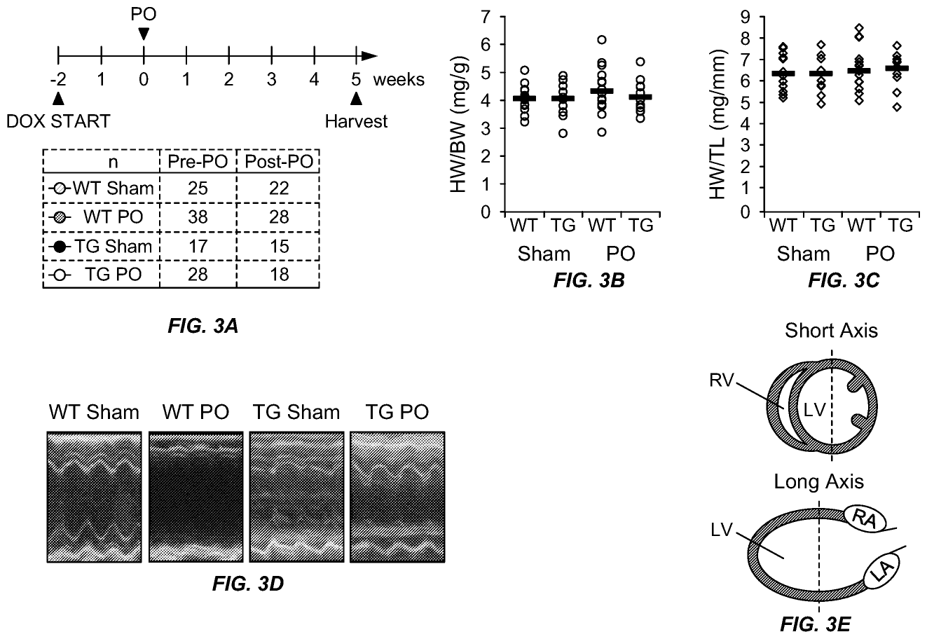

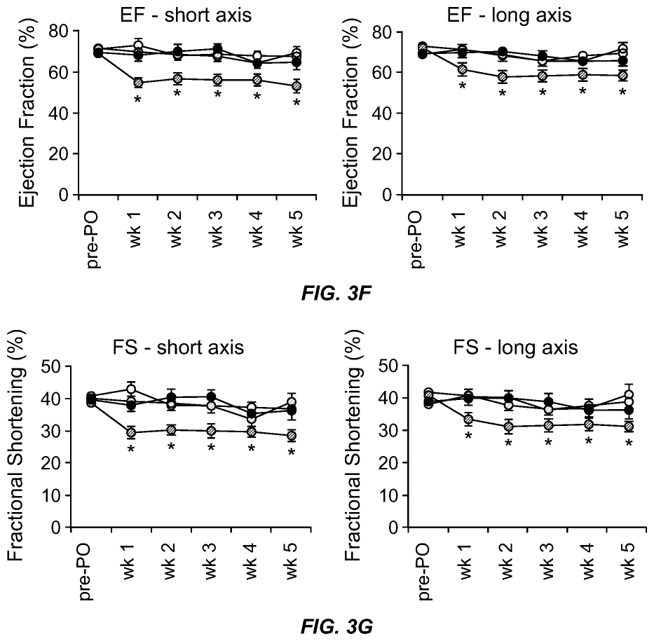

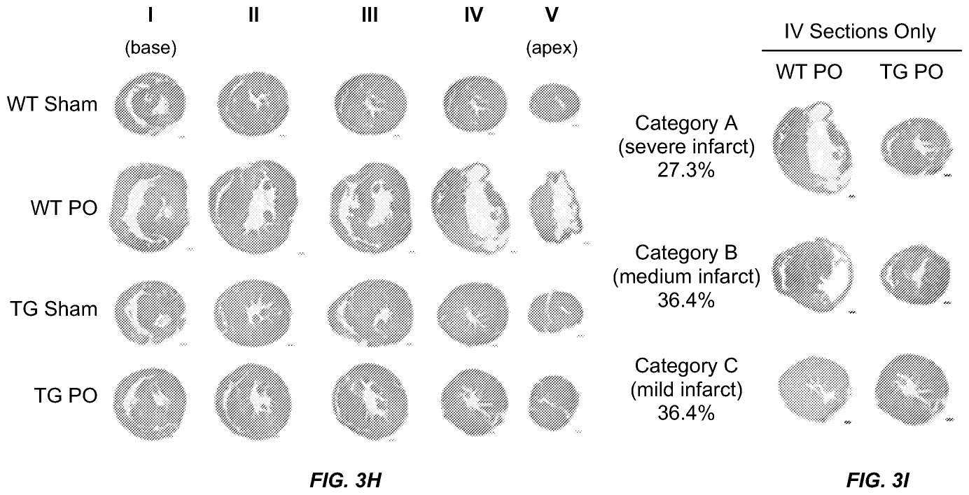

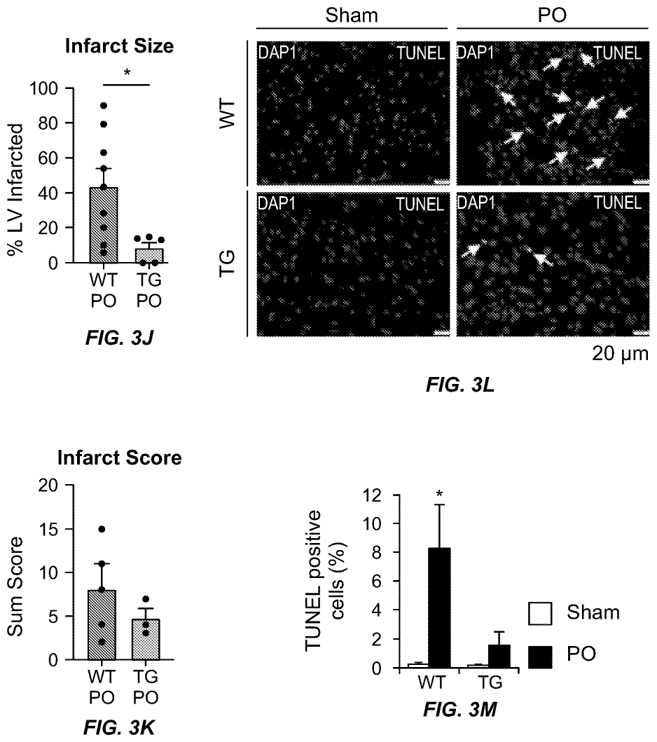

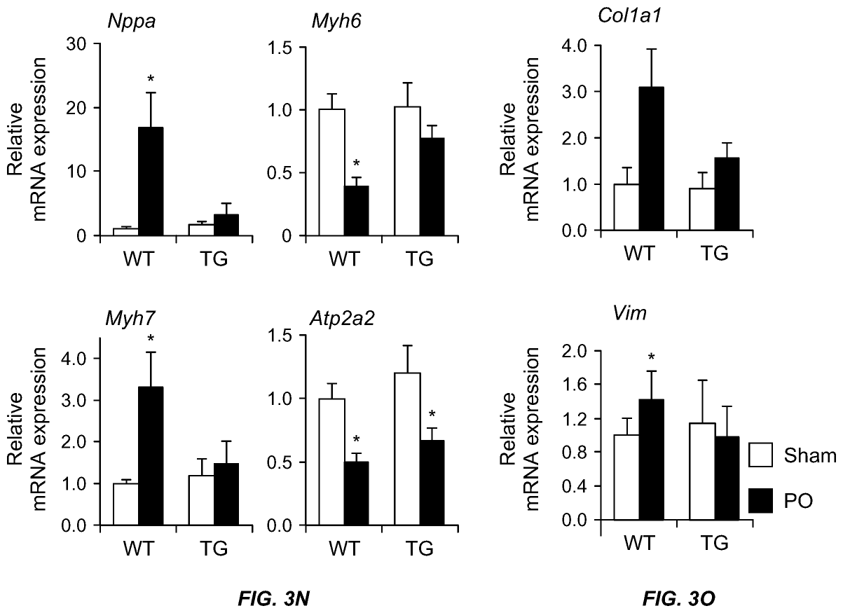

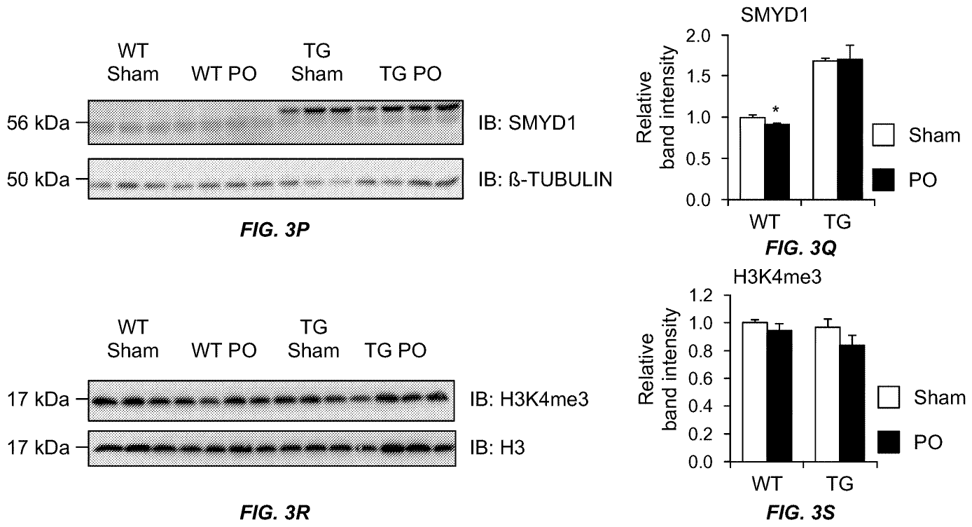

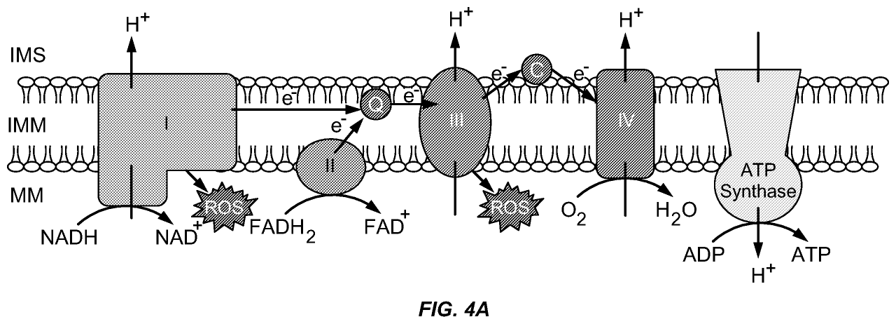

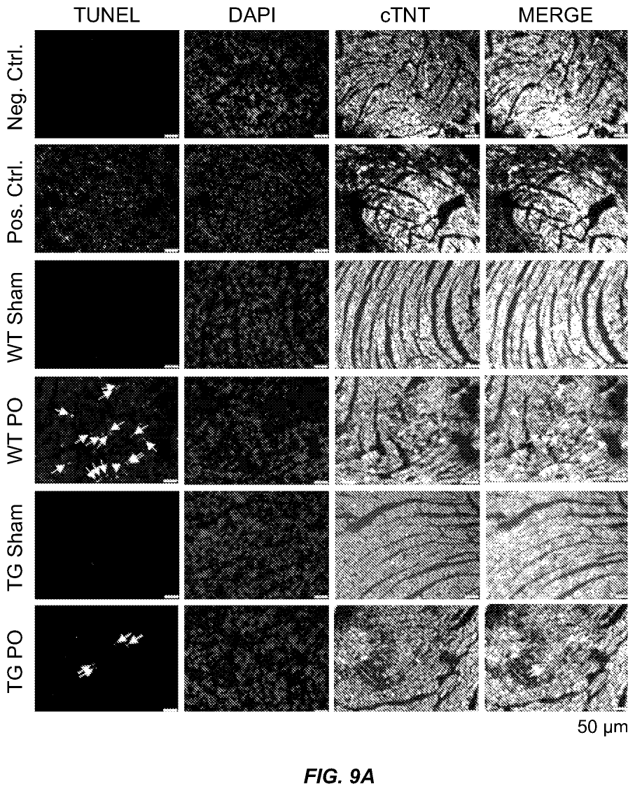

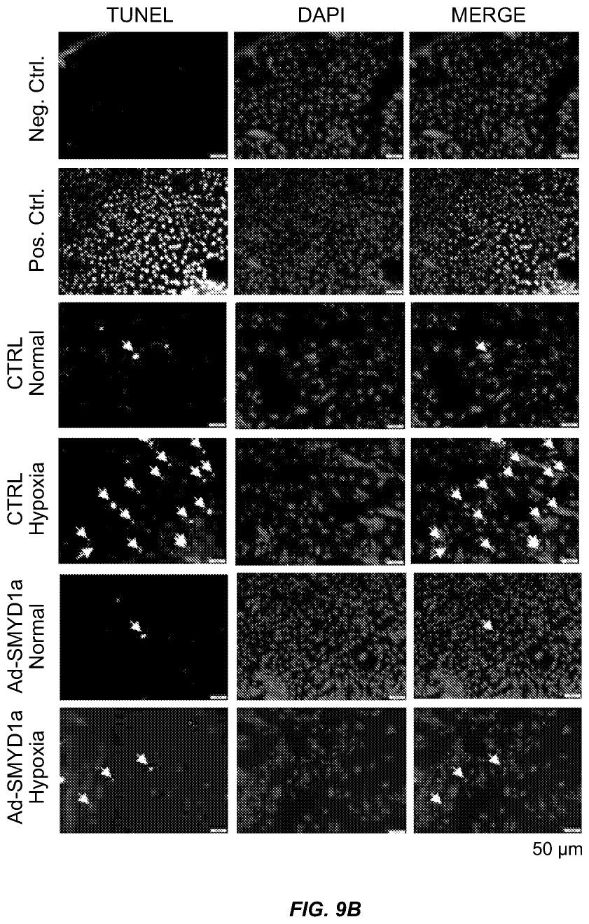

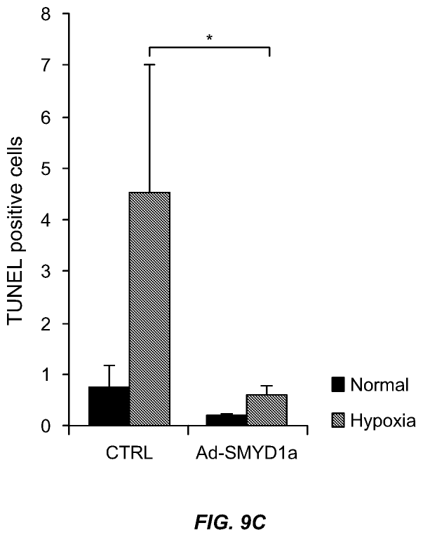

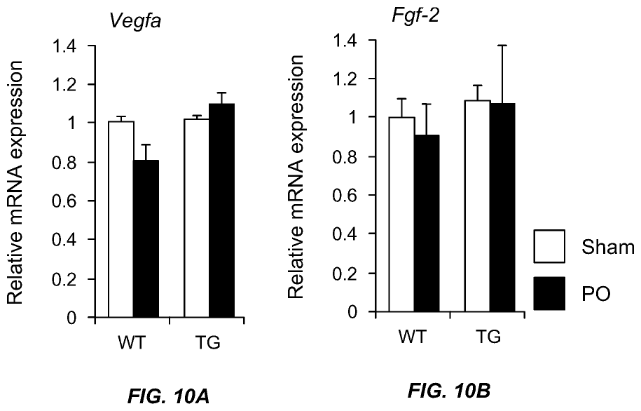

To assess the physiological effects of SMYD1 gain-of-function and evaluate if SMYD1 can attenuate disease-induced remodeling in an animal model, TG mice and their littermate controls (WT) were subjected to permanent occlusion (PO) of the LAD (or Sham) surgery. Mice were fed chow-containing doxycycline for 2 weeks prior to surgeries to induce overexpression of SMYD1 protein and were monitored weekly by echocardiography (FIG. 3A). Analysis of heart weight-body weight ratios (HW/BW) as well as tibia length-body weight (TL/BW) ratios five weeks after PO surgery revealed that there are no significant differences between WT and TG mice in response to LAD ligation (FIG. 3B, C). This is believed to be due to the combined effects of hypertrophic growth and cell death occurring at the same time but in different regions of the heart. Interestingly, the TG mice subjected to PO showed no decrease in ejection fraction (% EF) and fractional shortening (% FS) by echocardiography when compared to WT animals, which displayed a significant decrease in these measurements (FIG. 3D-G). The histological analysis showed reduced infarct size in cardiac tissue from TG mice (FIG. 3H). For this analysis, three qualitative categories of damage were defined in PO hearts to evaluate the variability of ischemic injury (FIG. 3I). Category A, observed in 27.3% of WT PO hearts, presented a severe phenotype, with more than half of the left ventricle forming a fibrotic scar; category B, observed in 36.4% of WT PO hearts presented a medium phenotype with equal amounts of fibrosis and cardiomyocytes in the left ventricle; and category C, observed in 36.4% of WT PO hearts presented a mild infarct, having a relatively small fibrotic scar with the majority of the left ventricle tissue remaining viable. However, when the severity of infarct was examined in TG PO mice, these animals showed little variability with 100% of samples characterized by a mild (category C) infarct. Three representative images (FIG. 3I) are included for these TG PO mice to highlight the lack of variability within this sample cohort. Furthermore, the infarct size was evaluated by first scoring tissue sections according to the extent of lesioned area and then measuring the midline infarct lengths and showed significant decrease in infarct size in TG PO mice as compared to WT PO group (FIG. 3J, K). In addition, the level of apoptosis was evaluated in transgenic mice which overexpress SMYD1 or in littermate controls (WT) 48 hours after permanent occlusion of the LAD or Sham surgery by TUNEL assay. The results of the tissue sections adjacent to the infarct area revealed a significant decrease in TUNEL-positive cells in TG mice in response to ischemic injury (FIG. 3L, M and FIG. 9A). To confirm, that the observed effect is cardiomyocyte-specific, cells were co-stained with cardiac troponin C (FIG. 9A). In addition, cultured H9c2 cardiomyoblasts that overexpress SMYD1 and were subjected to hypoxic conditions showed significant decrease in TUNEL-positive cells (FIG. 9B, C). Evaluation of markers of angiogenesis showed that overexpression of SMYD1 has no effect on the expression of Vegfa and Fgf-2 in TG mice and 48 h after permanent occlusion (FIG. 10). Overall, this data indicates that overexpression of SMYD1 protects from apoptosis in the ischemic heart.

Because overexpression of SMYD1 protects the heart from ischemic injury the expression of genes commonly dysregulated during cardiac disease was investigated in TG mice that were subjected to PO surgery. The gene expression analysis of these transcripts: Nppa, Myh6, Myh7 and Atpa2a2 (FIG. 3N) as well as markers associated with fibrosis: Vim. Colla (FIG. 3O) revealed significant changes following overexpression of SMYD1. In this data two key observations were seen: first, there was no observation of any significant changes in these transcripts at basal levels in WT and TG mice. Second, gene expression changes that are hallmarks of hypertrophy and failure in mice (i.e. increased Nppa) were unaffected in TG mice after PO, indicating that overexpression of SMYD1 inhibits disease-induced transcriptional changes (FIG. 3N, O).

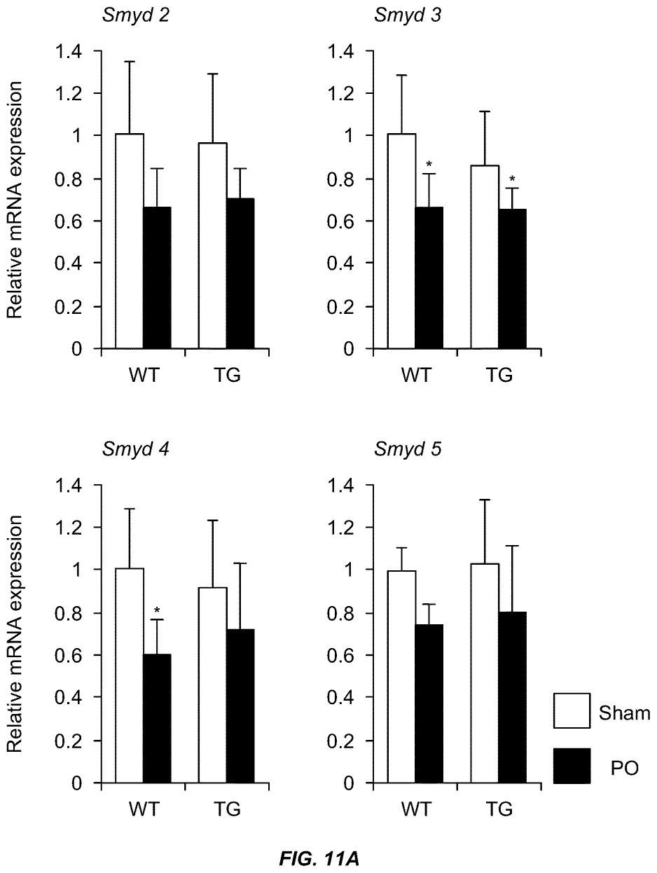

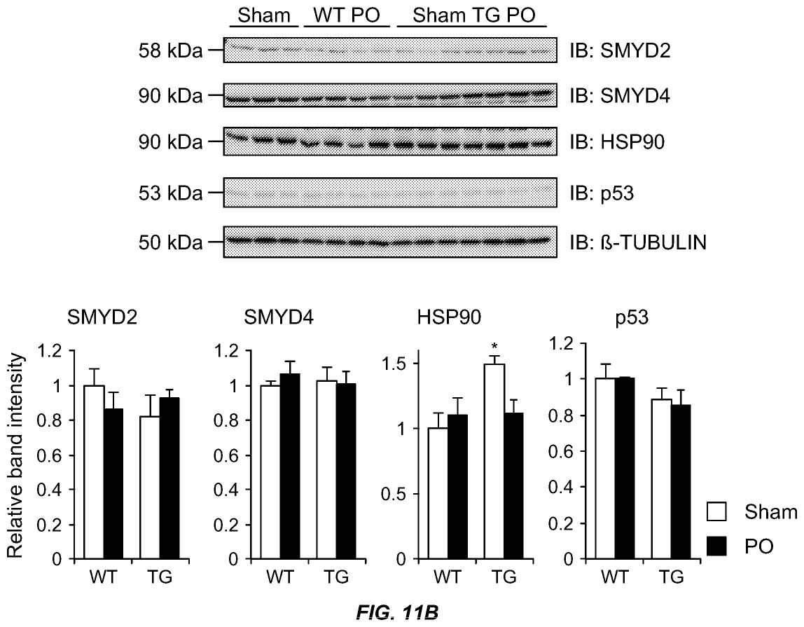

Evaluation of SMYD1 protein expression in TG mice subjected to permanent occlusion of the LAD showed similar levels of SMYD1 in TG mice five weeks after PO surgery (FIG. 3P, Q). Although there is no evidence to support interactions or molecular compensation between SMYD1 and other SMYD family members, their expression was examined in the TG mice at basal levels and after PO. SMYD1 overexpression does not affect transcript or protein expression levels of other SMYD family members, and only the Smyd3 transcript showed a modest decrease in response to permanent occlusion (FIG. 11A, B). In addition, expression of two proteins (p53 and HSP90) that have been shown to interact with SMYD1 was examined and only HSP90 was increased in TG mice (FIG. 11B).

Lastly, it was previously shown that SMYD1 trimethylates lysine K4 on histone H3, therefore it was investigated whether overexpression of SMYD1 affects global levels of this histone post-translational mark. Western blotting was performed for H3K4me3 in total cell lysates collected from TG and WT mice after PO or Sham surgeries and determined that overexpression of SMYD1 had no effect on global levels of histone H3K4me3 (FIG. 3R. S). Histone H3K4me3 can still be regulated by SMYD1 in this model at the individual gene level, even though no changes were seen in this post-translational mark globally. Methylation of histone H3K4 is a very dynamic process regulated by multiple methyltransferases and demethylases.

### 4. SMYD1 Overexpression Enhances Mitochondrial Respiration and ATP Production.

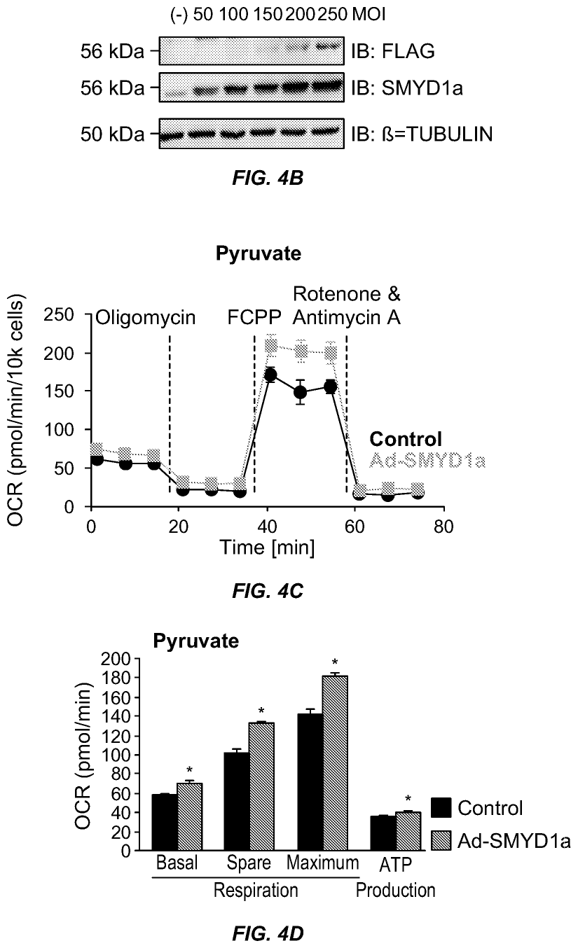

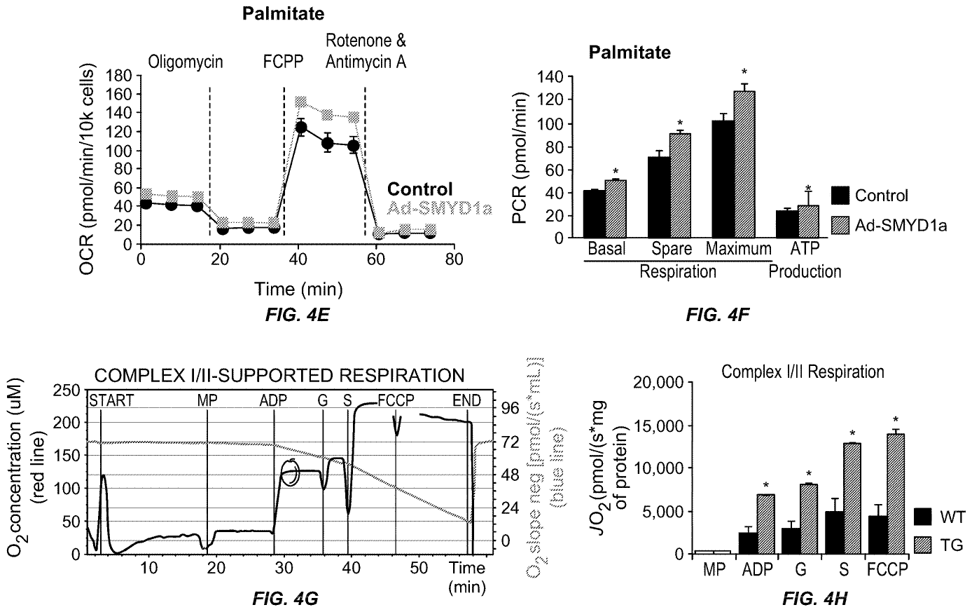

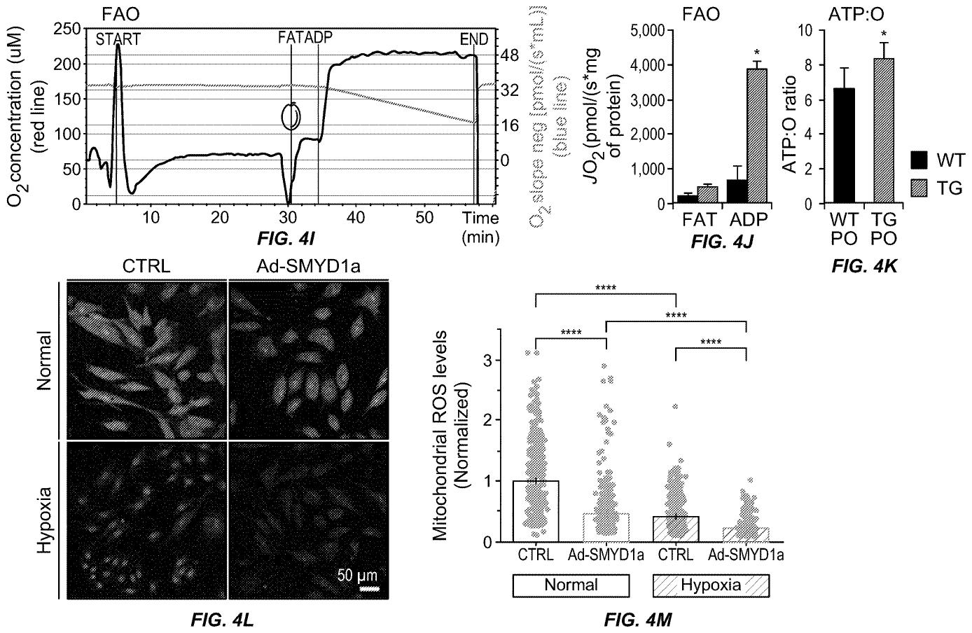

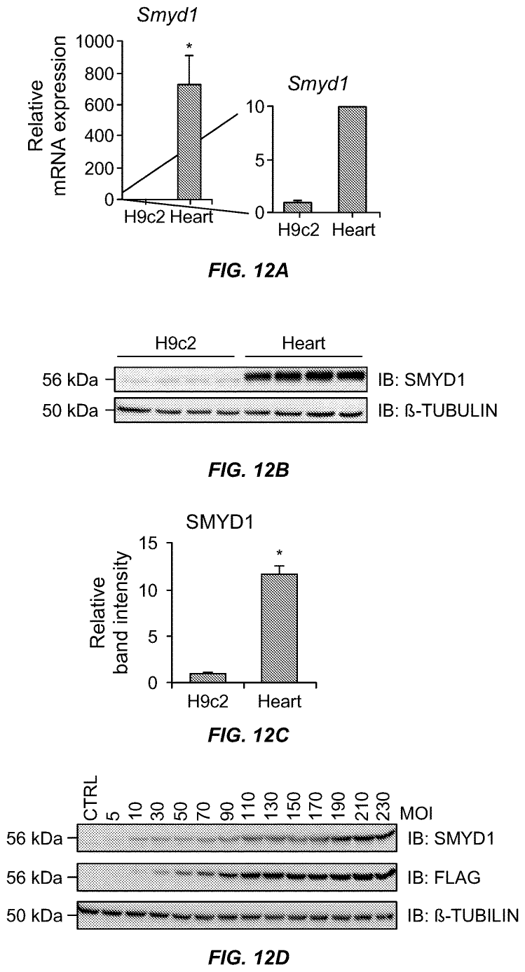

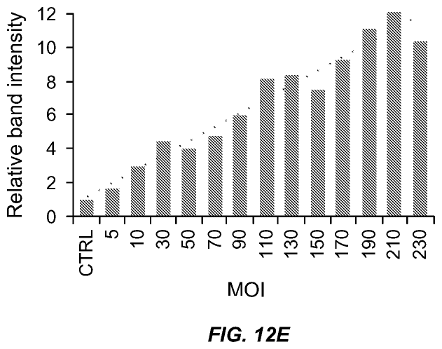

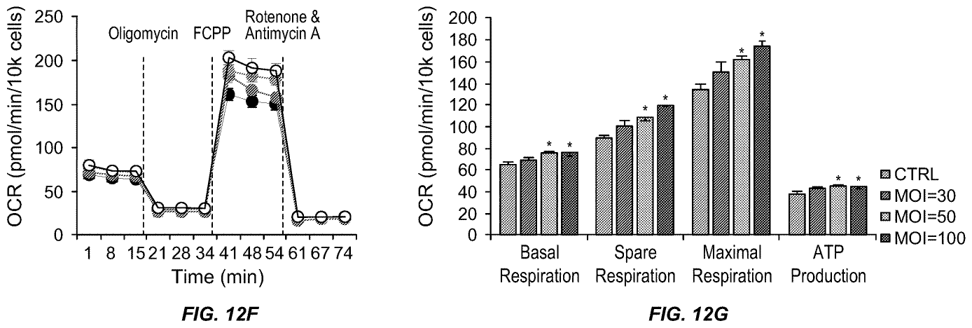

The electron transport chain is a cluster of four protein complexes in mitochondria that transfer protons through a membrane and drive the synthesis of ATP needed for essential cellular processes (FIG. 4A). The mammalian heart requires an enormous amount of ATP and deficits in ATP production have been linked to the development of heart failure and ischemic injury. Thus, to investigate the effects of SMYD1 overexpression on mitochondrial function a Cell Mito Stress Test was performed in H9c2 cardiomyoblasts transduced with adenovirus carrying SMYD1-FLAG (FIG. 4B). Overexpression of SMYD1 led to an increase in basal, spare, and maximal respiration capacity as well as increased ATP production when glucose or palmitate were used as the major respiration substrate (FIG. 4C-F). Next, using an Oroboros O2k oxygraph high-resolution mitochondrial respirometry was measured on mitochondria isolated from the transgenic and WT mice. Consistent with data in isolated cells, it was shown that SMYD1 overexpression leads to increased mitochondrial respiration through Complex I and II (FIG. 4G, H). In addition, when respiration was evaluated with substrates that support fatty acid oxidation (FAO) ~8-fold increase in 02 consumption was observed in TG mice (FIG. 4I, J). This data shows SMYD1 enhances mitochondrial oxidative capacity through both carbohydrate and FAO supported-ATP production. It is important to note, that there are significant differences in basal expression of SMYD1 between cultured H9c2 cardiomyoblasts and cardiac tissue (FIG. 12A-C). Thus, to demonstrate that the overexpression of SMYD1 in H9c2 cells is comparable to levels in TG mice were tested increasing MOI of adenovirus on SMYD1 protein levels (FIG. 12D, E) and oxygen consumption rates as measured in Cell Mito Stress Test (FIG. 12F-G). This data shows that when H9c2 cardiomyoblasts express SMYD1 at the same levels seen in adult cardiac tissue they display the same response to oxygen consumption and mitochondrial respiration seen in TG mice.

It has been previously reported that the failing heart loses its metabolic flexibility and can become energy deficient because of a decrease in its ability to produce ATP. To further assess oxidative phosphorylation (OXPHOS) efficiency in mitochondria isolated from WT and TG mice subjected to permanent occlusion of the LAD were evaluated ATP production rates 24 hours after PO by measuring molar amounts of ATP produced per mole of atomic oxygen consumed, known as the ATP:O ratio. Interestingly, an increase in the ATP:O ratio was observed in TG PO mice, indicating they have a higher respiratory efficiency (FIG. 4K). This enhanced respiratory efficiency would be particularly important during ischemic injury considering the oxygen supply would be limited in cardiac tissue.

The natural by-product of electron transport chain activity in mitochondria is production of reactive oxygen species, or ROS (FIG. 4A). Because a significant increase is seen in mitochondrial respiration in the TG mice, the effects of SMYD1 overexpression were investigated on ROS production. Cultured H9c2 cardiomyoblasts overexpressing SMYD1 were subjected to hypoxic conditions and imaged after staining with ROS-sensitive MitoSOX dye (FIG. 4L). The results show lower ROS production in cells that overexpress SMYD1 and after induced hypoxia (FIG. 4M).

### 5. SMYD1 Overexpression Maintains Metabolic Homeostasis by Regulating PGC-10 And its Downstream Targets.

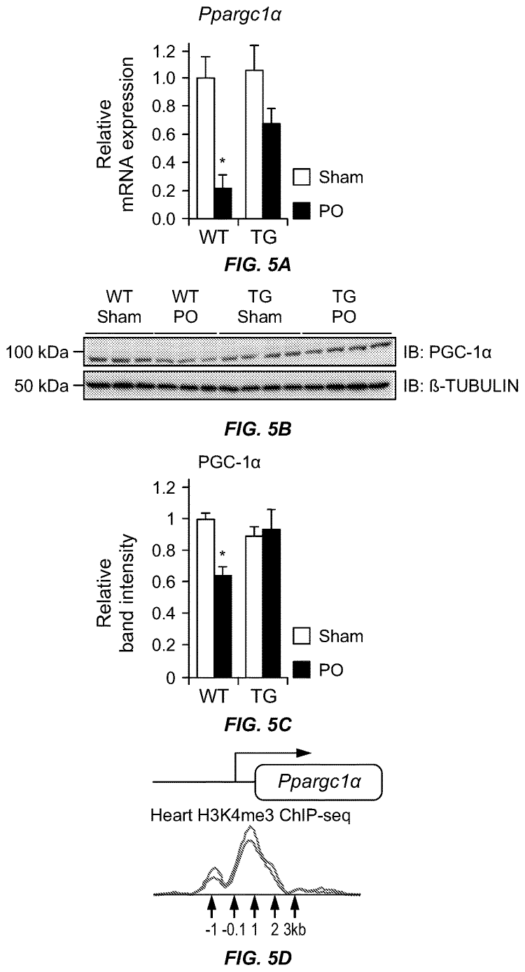

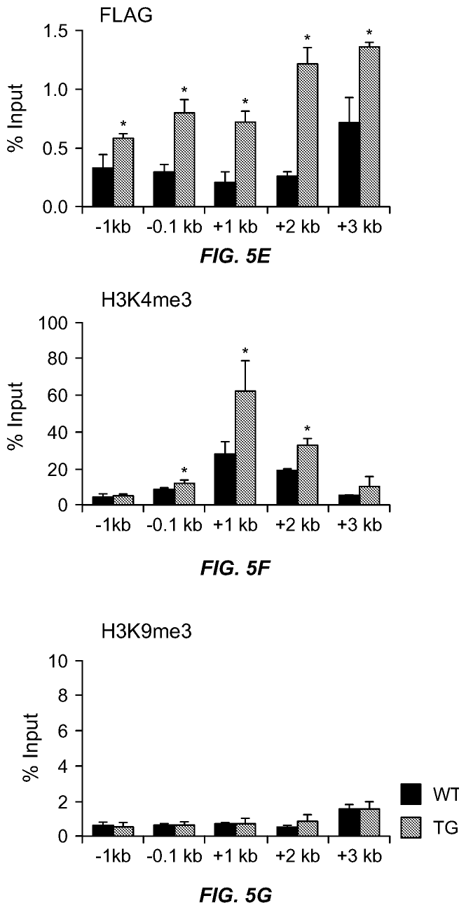

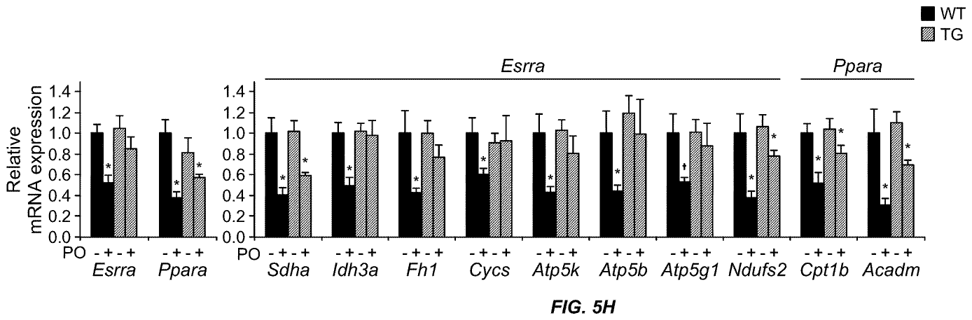

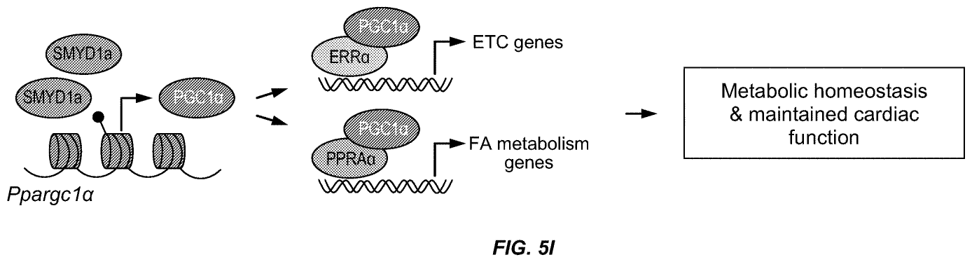

It has been shown that SMYD1 can regulate mitochondrial respiration in cultured cells, at least in part, by modulating the expression of PGC-1α, a master regulator of mitochondrial energetics. Loss of Smyd1 in the adult mouse heart leads to reduction of histone H3K4 trimethylation at the Ppargc1α promoter, however direct binding or methylation of the Ppargc1α promoter was never examined by SMYD1 in vivo. Therefore, in this study expression of PGC-1α was quantified via qPCR and western blotting in cardiac tissue from transgenic (TG) and control (WT) mice 48 hours after permanent occlusion and show that at this early time point PGC-1α levels are decreased (FIG. 5A-C), consistent with previous reports. Interestingly, however, increased expression of SMYD1 maintains PGC-1α expression at normal levels in the transgenic mice (FIG. 5A-C) but does not elevate it above basal levels. This is believed to be due to tightly controlled regulation of this gene and reflects not only the positive regulators of expression but also the homeostatic balance of negative regulators, e.g. demethylases.

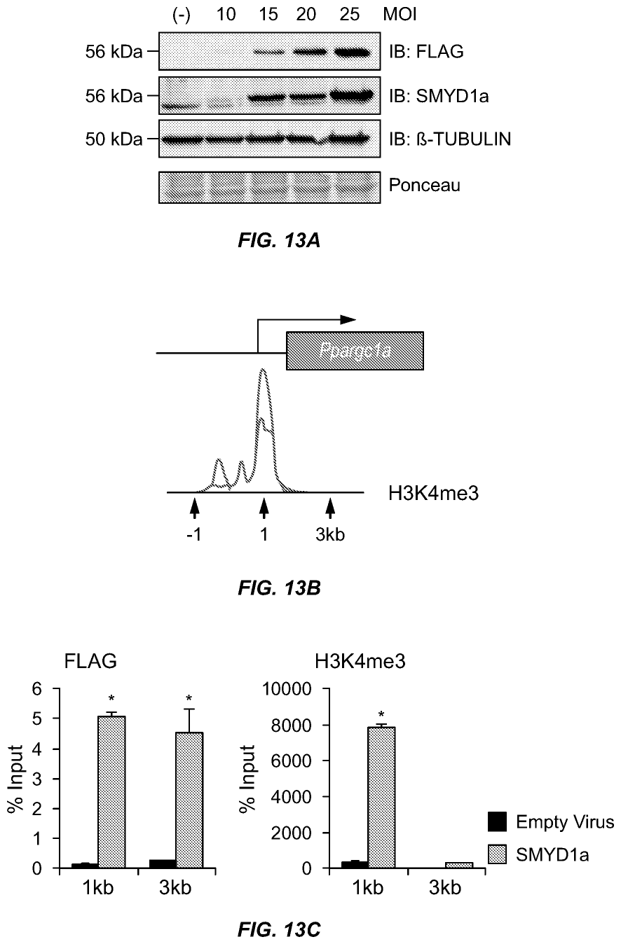

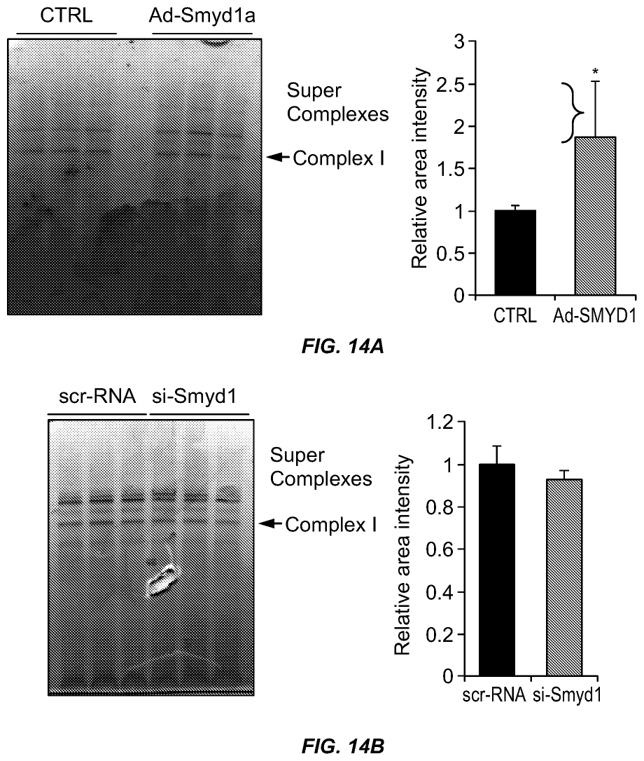

To determine whether the regulation of PGC-1α expression occurs in this model through direct binding of SMYD1 to the Ppargc1α promoter chromatin immunoprecipitation of FLAG-tagged SMYD1 was performed. Immunoprecipitated DNA was analyzed by ChIP-qPCR using primers that amplify the promoter region of Ppargc1α (FIG. 5D). For the first time in an animal model, it was demonstrated that SMYD1 binds to the Ppargc1α promoter and increases trimethylation of histone H3K4 (FIG. 5E-G). Additionally, this was confirmed this in isolated neonatal rat ventricular myocytes (NRVMs) via ChIP-qPCR by transducing cells with adenovirus carrying SMYD1-FLAG and observed binding and hypermethylation (H3K4me3) of the Ppargc1α promoter (FIG. 13A-C).

PGC-1α induces expression of metabolic genes through coactivation of two key transcription factors, ERR and PPARα, that directly regulate genes involved in fatty acid metabolism (through PPARα) or the TCA cycle and electron transport chain (through ERRα). Therefore, expression of these downstream targets of PGC-1α were examined in TG and WT mice. The data show that while the genes involved in OXPHOS and fatty acid oxidation are downregulated in response to ischemic injury, overexpression of SMYD1 maintains their expression at basal levels, including Sdha, Idh3a, Fh1, Cycs, Atp5k, Atp5b, Atp5g1, Ndufs2, Cpt1b, Acadm (FIG. 5H).

Overall, this data provide key insights into SMYD1's regulation of PGC-1α and confirm that maintaining PGC-1α expression is necessary for metabolic homeostasis (FIG. 5I), however it does not account for the increase in oxygen consumption in the SMYD1 transgenic mice. Therefore, there was interested in further examining key components of cardiac respiration in this model to identify the driver(s) of enhanced mitochondrial respiratory capacity.

### 6. SMYD1 Regulates ETC Supercomplex Formation and Cristae Morphology.

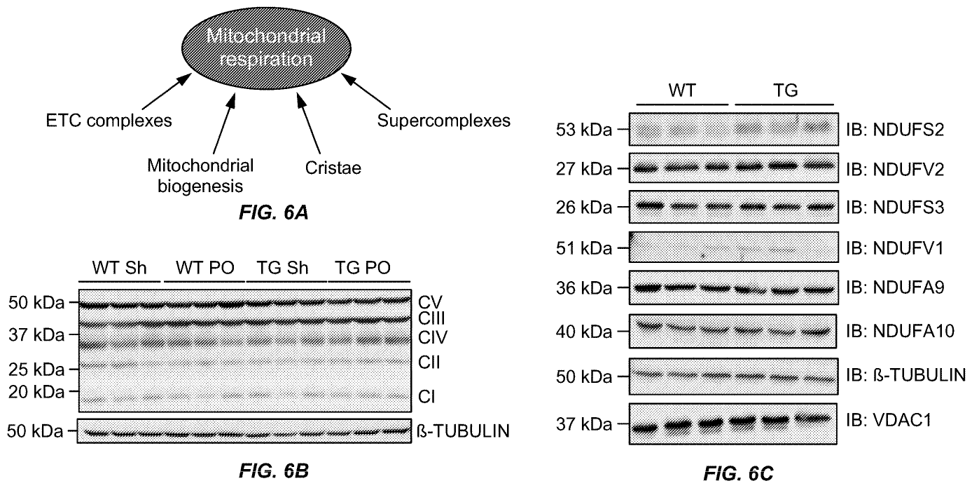

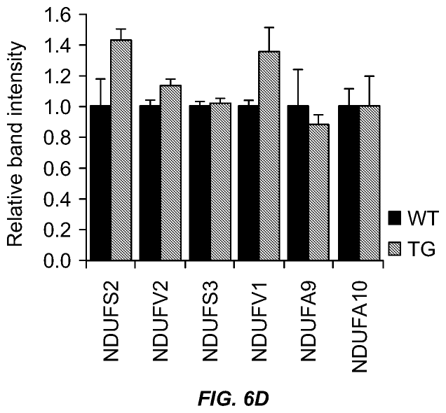

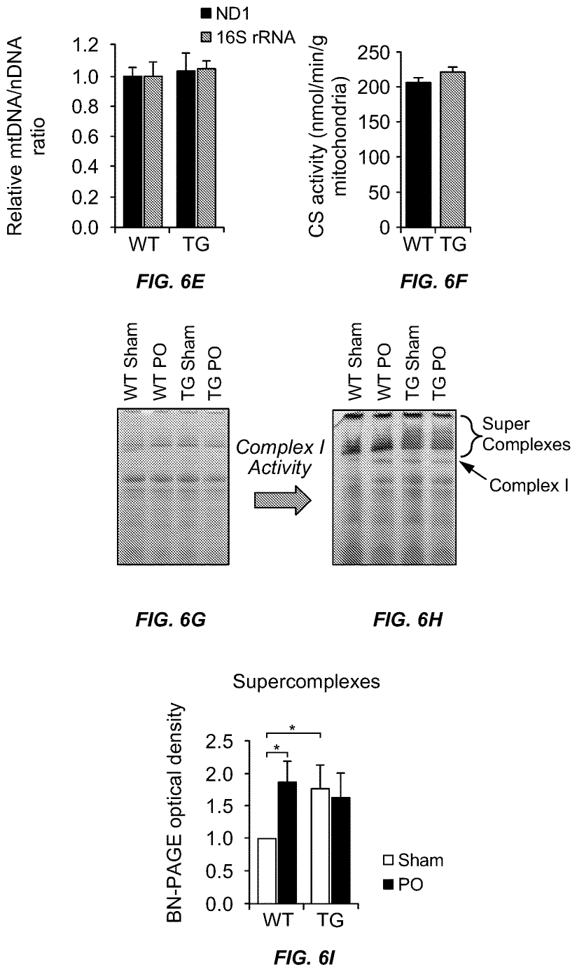

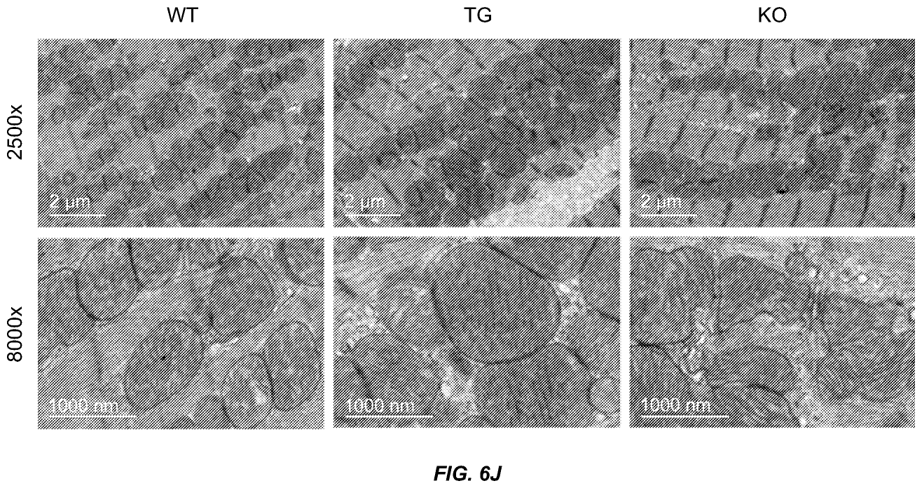

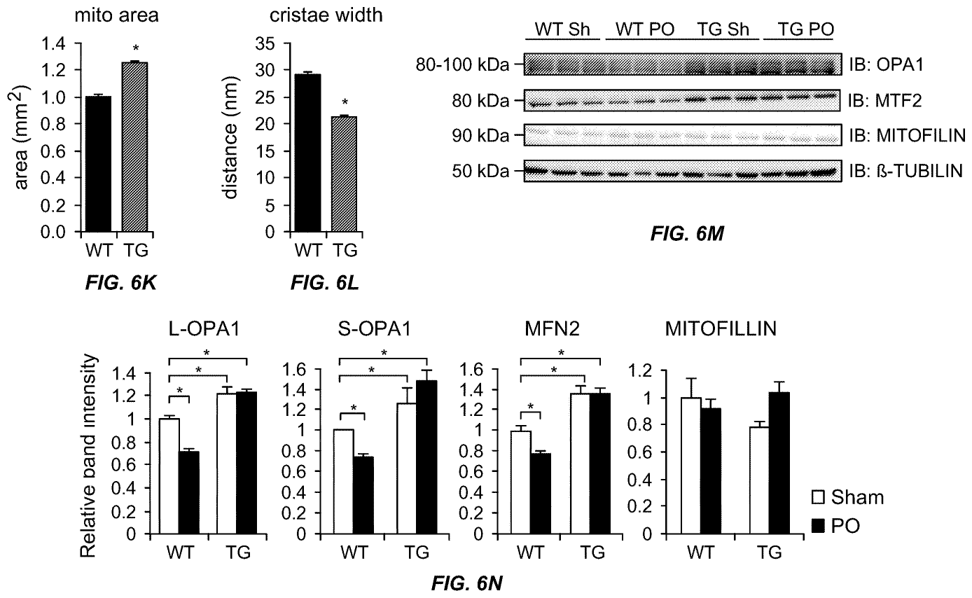

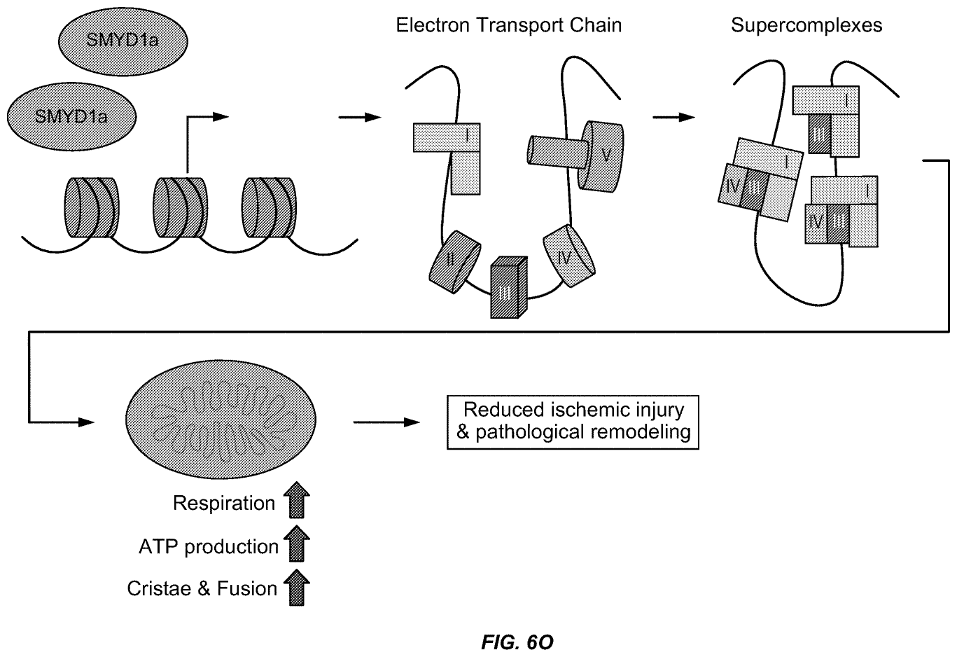

Mitochondrial respiration is the most important generator of energy in the cell. Abnormalities in this process lead to a variety of inherited and acquired cardiovascular diseases including ischemic heart disease. Several factors have been identified which positively influence mitochondrial respiratory capacity, including Electron Transport Chain (ETC) Complexes, cristae morphology, mitochondrial biogenesis, and ETC supercomplexes (FIG. 6A). To further examine mitochondrial function and elucidate the basis for the enhanced respiration in the TG mice, those components that positively affect respiration were systematically evaluated. First, the abundance of the ETC complex subunits was examined using an antibody cocktail containing one antibody for each of the five complexes and found no difference in the TG mice before or after PO (FIG. 6B). Additional immunoblotting of other ETC subunits showed no statistical difference in expression in TG mice (FIG. 6C, D).

To evaluate mitochondrial biogenesis in TG mice, three markers were quantified: 16S rRNA and ND1, which are encoded by mitochondrial DNA, and citrate synthase activity and showed no change in SMYD1 overexpression mice (FIG. 6E, F). This is consistent with, the high-resolution respirometry measurements, in FIG. 4, which were normalized to total mitochondrial content, indicating that the increased respiration is independent of mitochondrial content.

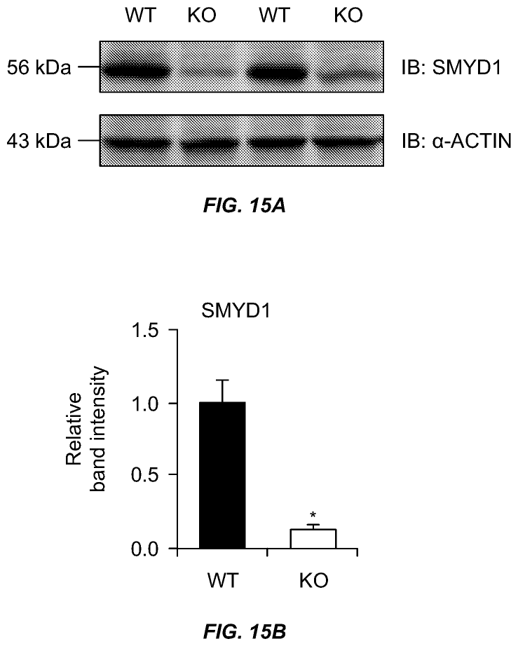

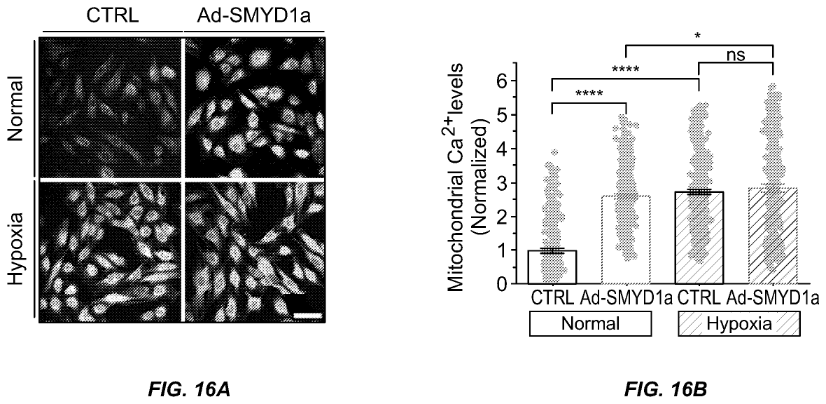

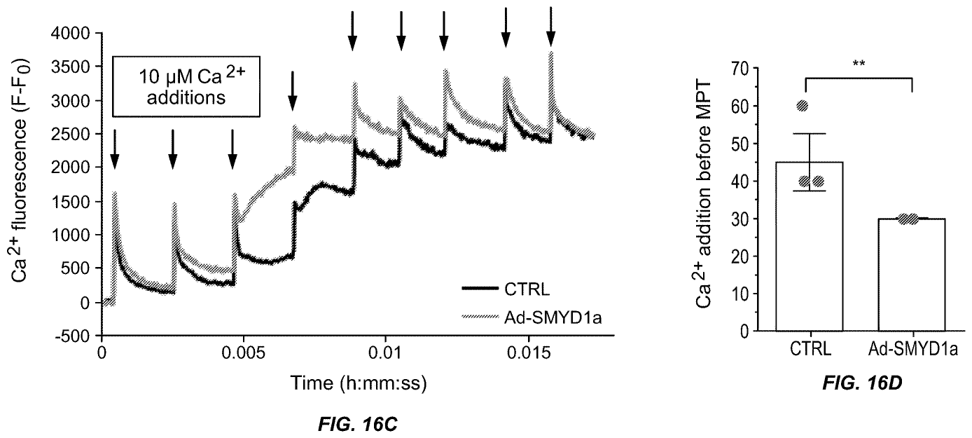

In response to a cell's energy demands, a certain portion of respiratory chain complexes can form higher-order structures called ‘supercomplexes’. These supercomplexes form within the folds of the inner mitochondrial membrane (known as cristae) and enhance respiration efficiency. Indeed, increased cristae formation has been shown to increase supercomplex formation. Therefore, to evaluate formation of electron transport chain supercomplexes, blue-native PAGE of mitochondria isolated from TG and WT mice we performed and showed increased supercomplex formation in transgenic mice at basal levels (FIG. 6G-I). The presence of these supercomplexes prior to ischemia may protect cardiac tissue from ischemic injury. The formation of ETC supercomplexes also increased 48 h after ischemic injury in WT animals, which is believed to be a part of a compensatory mechanism in response to the metabolic demands of the tissue. In addition, BN-PAGE of mitochondria isolated from H9c2 cardiomyoblasts that either overexpressed or lacked SMYD1 was performed and showed that overexpression of SMYD1 can increase formation of supercomplexes while loss of Smyd1 has no effect (FIG. 6A, B). Next, mitochondrial morphology was looked at via electron microscopy of cardiac tissue (FIG. 6J-L). Overexpression of SMYD1 resulted in larger mitochondria (FIG. 6J, K) with more dense, narrow cristae (FIG. 6L) as compared to WT controls, while loss of SMYD1 in Smyd1-KO mice (FIG. 15A-B) resulted in abnormal mitochondria with loss of cristae structure (FIG. 6J). The expression of proteins that regulate cristae morphology and mitochondria fusion was also analyzed. Western blot analysis showed that the known cristae remodeler OPA1 as well as the mitochondrial fusion protein MFN2 are upregulated in TG mice and after PO, but not mitofilin (FIG. 6M, N). Mitochondrial Ca2+ was analyzed and showed that overexpression of SMYD1 increased basal mitochondrial calcium but did little to alter calcium levels under hypoxic conditions. The increases in calcium levels were much lower in cells that overexpressed SMYD1 suggesting less calcium is taken up into the mitochondria during ischemia (FIG. 16). Overall, this data indicates that SMYD1 overexpression protects the myocardium from ischemic injury by enhancing mitochondrial respiration and ATP production through remodeling of cristae and formation of electron transport chain supercomplexes (FIG. 6O).

### 7. SMYD1 Regulates OPA1 Expression to Enhance Mitochondrial Respiration.

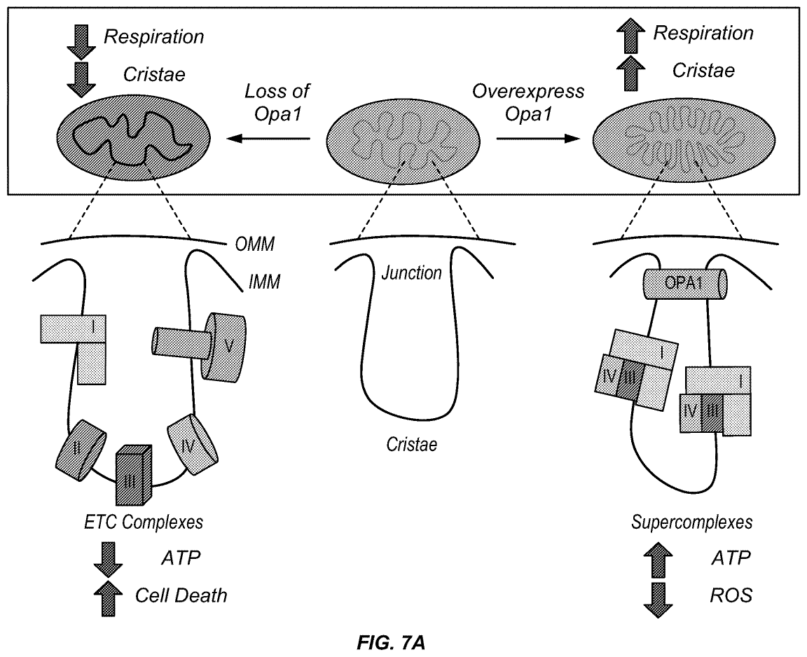

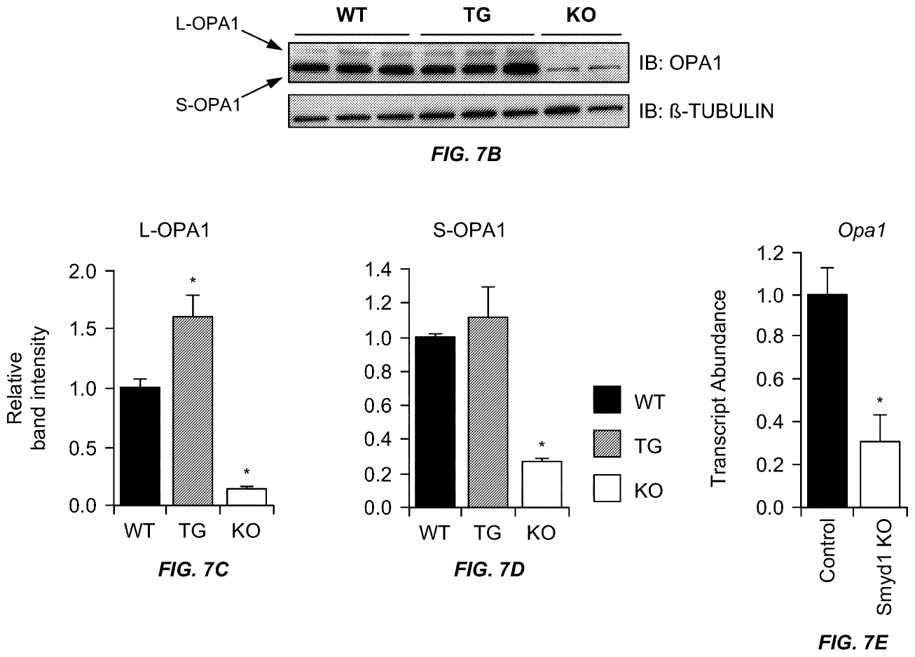

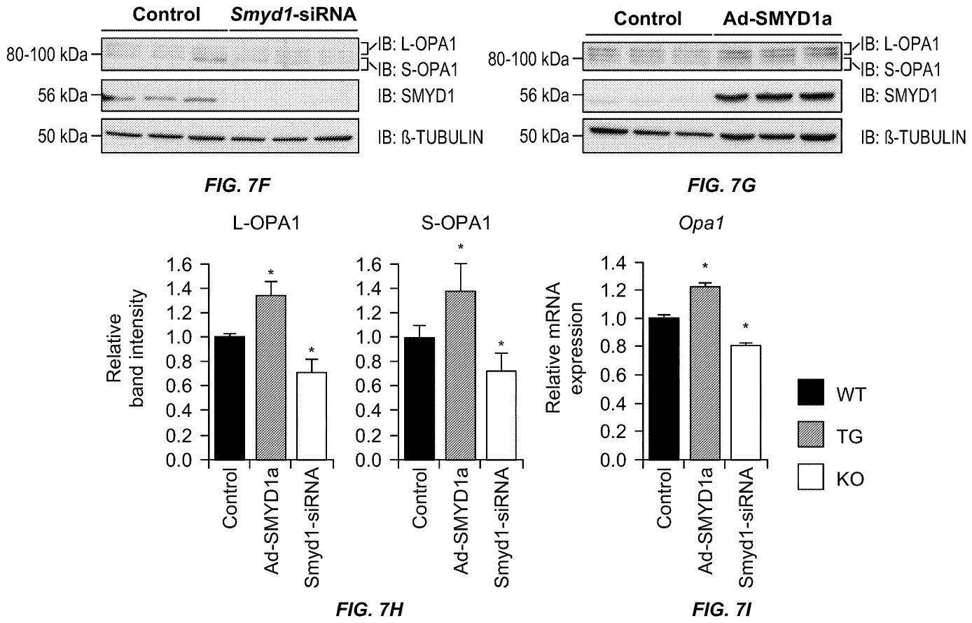

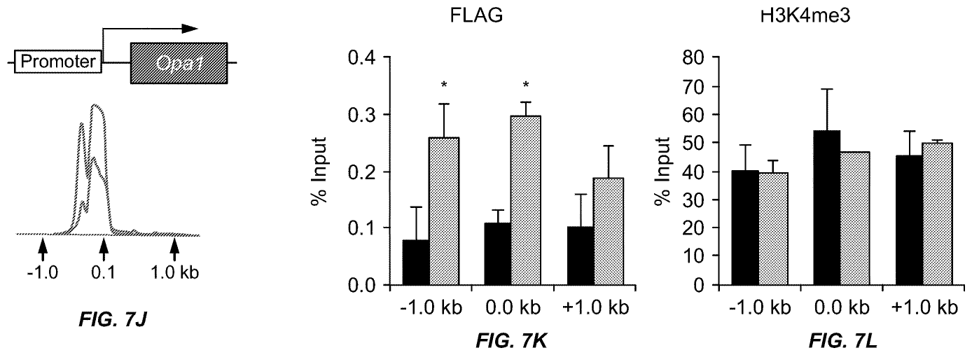

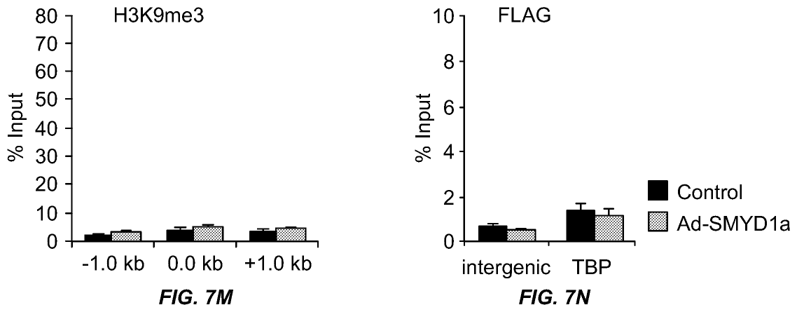

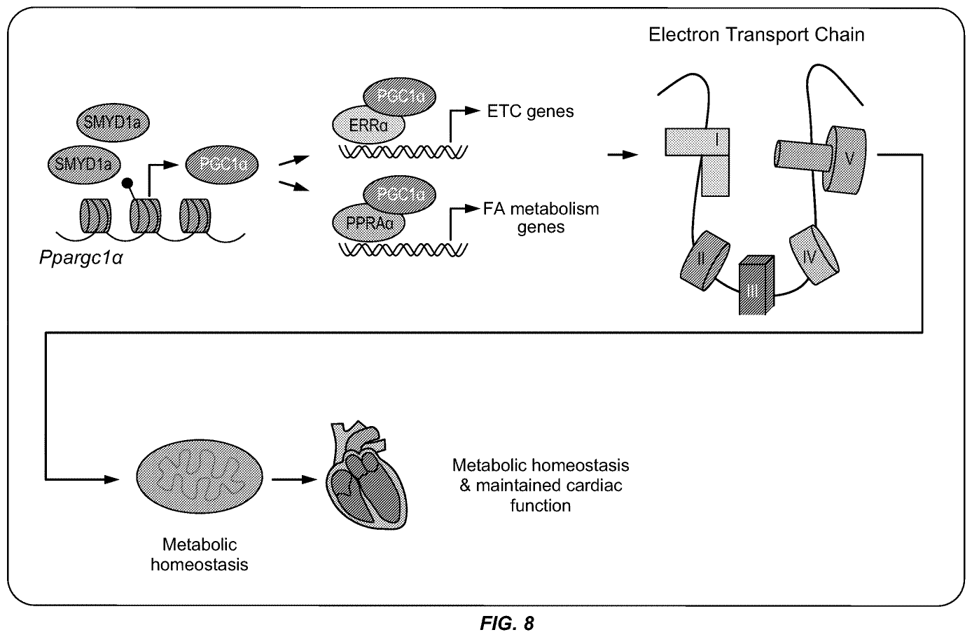

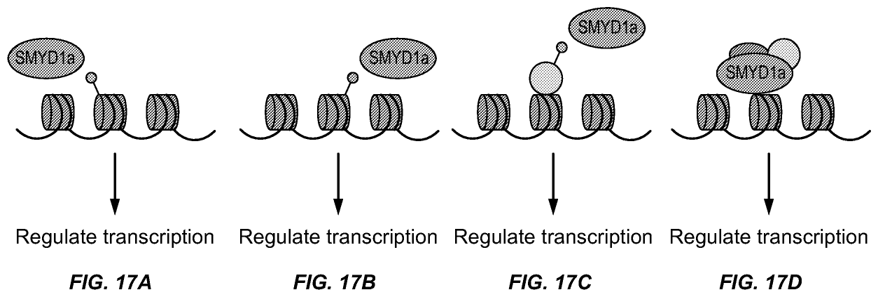

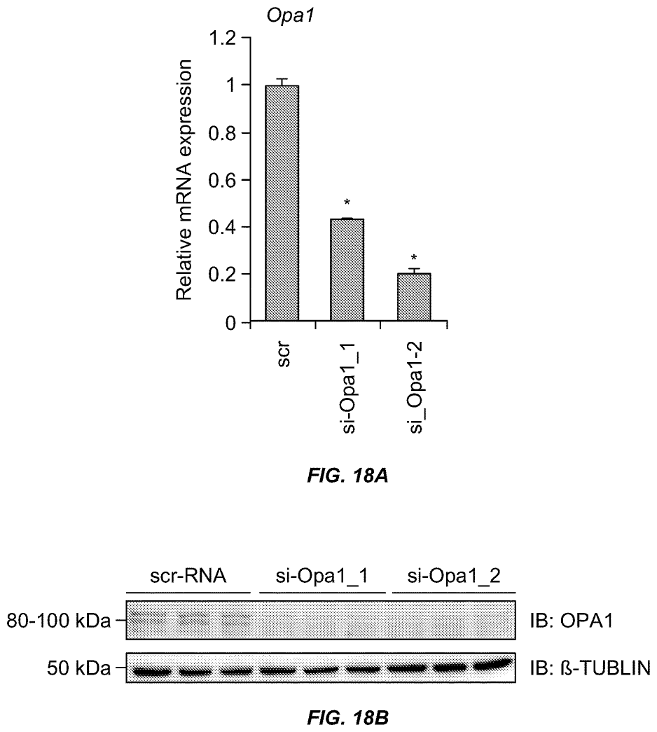

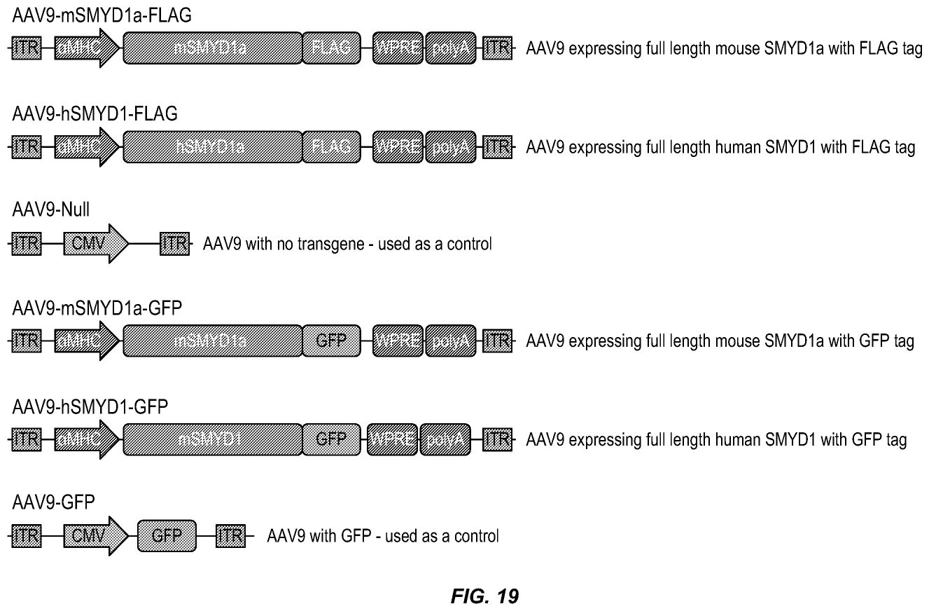

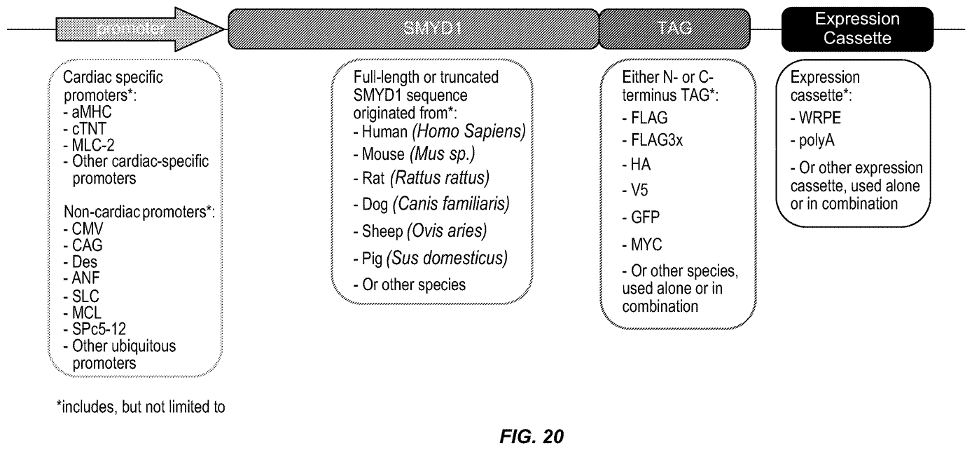

It has been previously reported that mitochondrial cristae shape determines the assembly and stability of respiratory chain supercomplexes and therefore the efficiency of mitochondrial respiration. A major regulator of mitochondrial fusion and cristae structure is the optic atrophy-1 protein (OPA1) which resides in the inner mitochondrial membrane. OPA1 has a direct effect on metabolism by influencing the formation and stability of electron transport chain complexes within the cristae. Expression of OPA1 is decreased in both human and rat heart failure and decreased in mice and cells subjected to hypoxia. In cells and animal models, loss of OPA1 leads to abnormal cristae morphology, decreased supercomplex formation and reduced respiration efficiency. Conversely, overexpression of OPA1 reduces infarct size after ischemic injury, increases mitochondrial fusion, cristae formation, respiration efficiency, supercomplex formation and ATP production (FIG. 7A). On a molecular level, OPA1 protein can exist in a long form or can be proteolytically processed to a short form by two peptidases: YME1L and OMA1. The specific functions of long and short OPA1 are still under investigation; however, increased abundance of long OPA1 can increase mitochondrial fusion and protect from cardiac dysfunction. Despite this post-transcriptional mode of regulation, there is currently no known epigenetic mechanism which regulates OPA1 expression. Interestingly, mice which overexpress OPA1 have a strikingly similar phenotype to the SMYD1 transgenic mice. Indeed, overexpression of SMYD1 resulted in larger mitochondria with more dense and narrower cristae, comparable to mice that overexpress OPA1. Therefore, levels of OPA1 were evaluated in the transgenic animals by western blotting. The results show increased long OPA1 in mitochondria from TG mice (FIG. 7B-D) and, conversely, a decrease in both long and short OPA1 forms in Smyd1-KO mice (FIG. 7B-E). In addition, OPA1 expression in H9c2 cardiomyoblasts subjected to knockdown or overexpression of SMYD1 were examined and a loss of Smyd1 that lead to down-regulation of OPA1 was observed, whereas SMYD1 overexpression increases OPA1 (FIG. 7F-I). Further, to investigate the mechanism by which SMYD1 controls OPA1 expression, the Opal loci was examined using ChIP-qPCR in cultured H9c2 cardiomyoblasts (FIG. 7J-N). Significant enrichment of SMYD1 binding at the Opal promoter was observed (FIG. 7K), however it did not correlate with enrichment of histone H3K4 trimethylation across the same region (FIG. 7L), indicating SMYD1 regulates expression of this gene through a methyltransferase-independent mode, as has been seen with other methyltransferases (FIG. 17). To affirm that SMYD1 regulates mitochondrial respiration through transcriptional control of OPA1, mitochondrial respiratory capacity was analyzed and the Cell Mito Stress Test was conducted in H9c2 cardiomyoblasts. Decreased mitochondrial respiration in Opal-KD cells, as reflected in lower basal, spare and maximal respiration and ATP production was observed (FIG. 7O, P, FIG. 18). As expected, overexpression of SMYD1 led to increased respiration capacity, but most importantly, overexpression of SMYD1 in the absence of Opal did not rescue basal, maximal and spare respiration or ATP production (FIG. 7O. P). Overall, this data shows that SMYD1 regulates the expression of OPA1 in the heart.

### D. Discussion

The data disclosed herein shows 1) a demonstration of SMYD1's ability to positively regulate cardiac energetics in an animal model and protect from ischemic injury and 2) the identification of OPA1 as a novel downstream target of this myocyte-specific methyltransferase. To delineate how SMYD1 regulates energy efficiency and metabolism in the cardiomyocyte, a mouse model capable of inducible cardiomyocyte-specific SMYD1 overexpression was generated. When subjected to ischemic injury these transgenic mice display reduced infarct size and cardiomyocyte death concomitant with enhanced mitochondrial respiratory efficiency. In addition, the molecular analysis revealed that the cardiac tissue in these animals is protected from ischemic injury through SMYD1's synergistic regulation of two key mitochondrial pathways. First, SMYD1 maintains metabolic homeostasis by preserving basal expression of PGC-1α and its downstream targets including electron transport chain subunits. Second, SMYD1 regulates expression of OPA1, a key regulator of cristae morphology and ETC supercomplex formation by which it enhances mitochondrial respiration and ATP production.

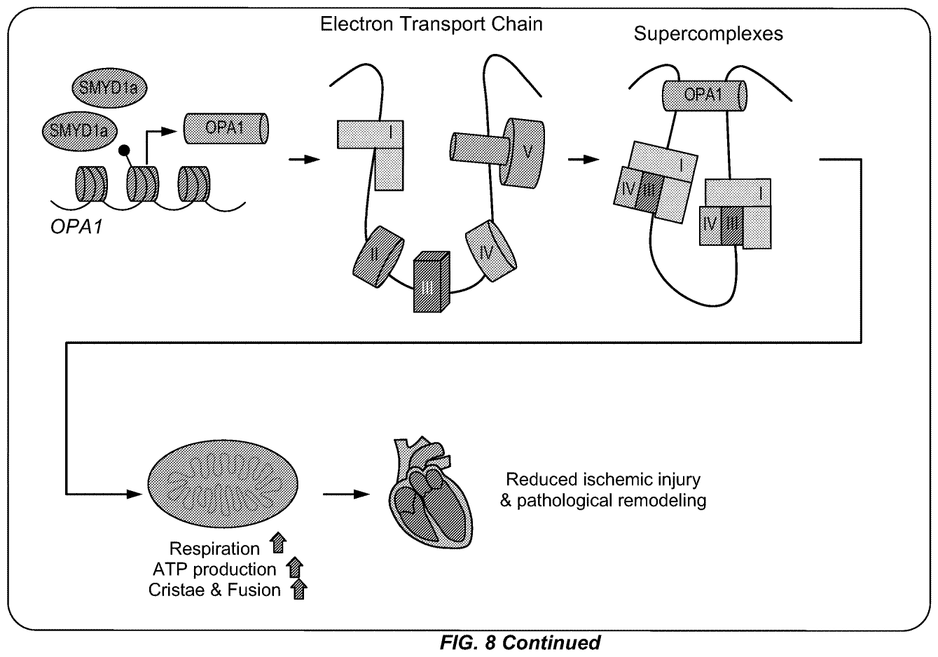

In the heart, it has been established that PGC-1α regulates cardiac energetics and that its expression is decreased in the mouse heart after pressure overload-induced hypertrophy and in isolated cardiomyocytes treated with hypertrophic agonists. This decrease in PGC-1α expression also corresponds to a decrease in PGC-1α target genes. In the failing human heart, PGC-1α expression has been shown to vary with most reports detecting a decrease and others showing no change. This variability is likely due to the time points examined, therapeutic interventions administered and the presence of other comorbidities (including diabetes and obesity), not present in animal models. In addition, the expression of PGC-1α has also been shown to be critical for maintaining metabolic homeostasis where significant changes in expression (either increasing or decreasing PGC-1α expression) have been detrimental to cardiac function in mouse models. Therefore, maintaining basal levels of PGC-1α may be a key mechanism to maintain myocyte respiration and ATP production. While several transcription factors have been identified which can influence PGC-1α expression, the epigenetic mechanisms, which underlie transcriptional regulation of PGC-1α, are only beginning to be identified. Specifically, SMYD1 was identified as the only known epigenetic regulator of PGC-1α expression in the cardiomyocyte. In the transgenic model presented here, it was shown that SMYD1 can regulate PGC-1α expression during ischemic injury. It's interesting to note that increased SMYD1 does not increase PGC-1α expression beyond basal levels, which were believed to be due to precisely controlled regulation (FIG. 8) and reflects the homeostatic balance between positive regulators of expression (like histone methyltransferases) and negative regulators (like histone demethylases). Indeed, previous reports have shown that increase or decrease in PGC-1α in the heart leads to metabolic imbalances and cardiac dysfunction, highlighting the importance of maintaining normal levels. However, while the data provide key molecular insights into SMYD1's regulation of PGC-1α and confirm that maintaining PGC-1α expression is necessary for metabolic homeostasis, it alone does not account for the increase in respiration capacity in the SMYD1 transgenic mice at physiological conditions.

Further evaluation of the TG mice demonstrated that the increased oxygen consumption is due to increased supercomplex formation of ETC complexes. These changes are influenced by increased mitochondrial cristae density, which is driven by OPA1, a master regulator of mitochondrial fusion and cristae morphology. In addition, reciprocal regulation of OPA1 expression is seen in both the SMYD1 transgenic mice (where OPA1 is increased) and the Smyd1 knockout mice (where OPA1 is decreased). Moreover, the phenotype in the SMYD1 gain-of-function mice is strikingly similar to the phenotype of transgenic mice which overexpress OPA1 where mild overexpression of OPA1 (1.6-fold increase) leads to increased cristae density, narrowing cristae junctions, increased supercomplex formation, enhanced respiration and protection from ischemic injury. Overall, the results show OPA1 as a downstream target of SMYD1 and reveal a novel epigenetic pathway by which cardiomyocytes regulate cristae structure to dynamically adapt to energy demands. The data clearly indicates that SMYD1 directly regulates OPA1 expression and demonstrates direct recruitment of SMYD1 to the Opal promoter region. However, this binding does not correlate with enrichment of the histone H3K4me3 at the same site but may imply regulation by a different mechanism (FIG. 17). The canonical model regarding transcriptional regulation by methyltransferases indicates that each methyltransferase modifies a single, nonrandom, and specific amino acid residue on histones and influences transcription in only one direction: by either activating or repressing gene expression (FIG. 17A). However, the most recent review of the literature indicates that the same methyltransferase can also modify different histone residue(s) to both activate or repress transcription (FIG. 17B). In addition, some methyltransferases can also regulate gene expression through methylation of non-histone proteins, including transcription factors, chromatin remodeling proteins or other components of transcriptional machinery that regulate chromatin structure and its accessibility (FIG. 17C). Finally, some methyltransferases have been shown to regulate transcription in a methyltransferase-independent mechanisms where they interact with (and do not methylate) other components of a chromatin binding complex (FIG. 17D). Therefore, the transcriptional regulation of OPA1 by SMYD1 could occur through either, 1) a methyltransferase-independent mechanism (which has been observed in other methyltransferases) or 2) by SMYD1 methylating a lysine residue other than K4 on histone H3 in the Opal promoter, although to date no other lysine residues on histones have been shown to be methylated by SMYD1.

In conclusion, the data presented here provide a mechanistic basis for understanding how SMYD1's synergistic regulation of PGC-1α and OPA1 maintains expression of electron transport chain subunits and remodels cristae structure, respectively, to enhance respiration and protect the heart from ischemic injury (FIG. 8). Ultimately, SMYD1 is highlighted as an epigenetic regulator of mitochondrial respiration and provide broad implications for understanding how the cardiomyocyte upregulates energy efficiency to dynamically adapt to energy demands of the cell.

Those skilled in the art will recognize, or be able to ascertain using no more than routine experimentation, many equivalents to the specific aspects of the method and compositions described herein. Such equivalents are intended to be encompassed by the following claims.

We claim:

1. A method of reducing infarct size in a subject comprising increasing SMYD1 in the subject.

2. A method of reducing myocyte death in a subject comprising increasing SMYD1 in the subject.

3. The method of any one of claims 1-2, wherein increasing SMYD1 comprises increasing Smyd1 expression.

4. The method of any one of claims 1-2, wherein increasing SMYD1 in the subject comprises administering a therapeutically effective amount of SMYD1 to the subject.

5. The method of any one of claims 1-3, wherein increasing SMYD1 in the subject comprises administering a therapeutically effective amount of a vector comprising Smyd1 to the subject.

6. The method of claim 5, wherein the vector is a viral vector.

7. The method of claim 6, wherein the viral vector is an adeno-associated viral vector.

8. The method of any one of claims 6-7, wherein the vector comprises an inducible promoter.

9. The method of any one of claims 1-8, wherein increasing SMYD1 in the subject comprises administering a therapeutically effective amount of a composition that increases smyd1 expression.

10. A method of protecting a heart from ischemic injury comprising increasing SMYD1 in the heart.

11. The method of claim 10 wherein increasing SMYD1 in the heart comprises administering SMYD1 to the heart.

12. The method of any one of claims 10-11, wherein increasing SMYD1 in the heart comprises administering a therapeutically effective amount of SMYD1 to the heart.

13. The method of any one of claims 10-11, wherein administering comprises administering a therapeutically effective amount of a vector comprising smyd1 to the heart.

14. The method of claim 13, wherein the vector is a viral vector.

15. The method of claim 14, wherein the viral vector is an adeno-associated viral vector.

16. The method of any one of claims 14-15, wherein the vector comprises an inducible promoter.

17. The method of any one of claims 10-16, wherein the heart is in a subject.

18. The method of claim 17, wherein administering comprises intravenous administration.

19. The method of any one of claims 10-18, wherein increasing SMYD1 in the subject comprises administering a therapeutically effective amount of a composition that increases smyd1 expression.

20. The method of any one of claims 1-19, wherein there is a >50% reduction in infarct size.

21. The method of any one of the preceding claims, wherein there is decreased myocyte cell death.

22. A method of enhancing mitochondrial respiration in a cell comprising increasing SMYD1 in the cell.

23. The method of claim 22, wherein the SMYD1 increases expression of OPA1 and the OPA1 enhances mitochondrial respiration.

24. The method of claim 22 or 23, wherein enhanced mitochondrial respiration is an increase in mitochondrial cristae formation.

25. The method of any one of claims 22-24, wherein there is a stabilization of respiratory chain supercomplexes within cristae of the mitochondria.

26. The method of any one of claims 22-25, wherein increasing SMYD1 comprises increasing smyd1 expression.

27. The method of any one of claims 22-26, wherein increasing SMYD1 in the cell comprises administering a therapeutically effective amount of SMYD1 to the cell.

28. The method of claim 27, wherein administering comprises contacting the cell with a vector comprising SMYD1.

29. The method of claim 28, wherein the vector is a viral vector.

30. The method of claim 29, wherein the viral vector is an adeno-associated viral vector.

31. The method of any one of claims 22-30, wherein the cell is in a subject.

32. The method of claim 31, wherein administering comprises intravenous administration of a vector comprising SMYD1 to the subject.

33. A method of increasing ATP production in a cell comprising increasing SMYD1 in the cell.

34. The method of claim 33, wherein increasing SMYD1 comprises increasing smyd1 expression.

35. The method of any one of claims 33-34, wherein increasing SMYD1 in the cell comprises administering a therapeutically effective amount of SMYD1 to the cell.

36. The method of claim 35, wherein administering comprises contacting the cell with a vector comprising SMYD1.

37. The method of claim 36, wherein the vector is a viral vector.

38. The method of claim 37, wherein the viral vector is an adeno-associated viral vector.

39. The method of any one of claims 33-38, wherein the cell is in a subject.

40. The method of claim 39, wherein administering comprises intravenous administration of a vector comprising smyd1 to the subject.

41. The method of any one of the preceding claims, wherein increasing SMYD1 in the subject or heart is compared to levels of SMYD in the subject or heart of healthy subjects.

42. A vector comprising a nucleic acid construct, wherein the nucleic acid construct comprises a nucleic acid sequence capable of encoding SMYD1.

43. The vector of claim 42, wherein the vector is a viral vector.

44. The vector of claim 43, wherein the viral vector is an adeno-associated viral (AAV) vector.

45. The vector of any one of claims 42-44, wherein the nucleic acid sequence capable of encoding SMYD1 comprises the sequence
AGTGTTAAATAACTGCCGCGCTGGCCTGACAGTCTCTGAGATGACAATAG
GGAGAATGGAGAACGTGGAGGTCTTCACCGCTGAGGGCAAAGGAAGGGG
TCTGAAGGCCACCAAGGAGTTCTGGGCTGCAGATATCATCTTTGCTGAGC
GGGCTTATTCCGCAGTGGTTTTTGACAGCCTTGTTAATTTTGTGTGCCACA
CCTGCTTCAAGAGGCAGGAGAAGCTCCATCGCTGTGGGCAGTGCAAGTTT
GCCCATTACTGCGACCGCACCTGCCAGAAGGATGCTTGGCTGAACCACAA
GAATGAATGTTCGGCCATCAAGAGATATGGGAAGGTGCCCAATGAGAAC
ATCAGGCTGGCGGCGCGCATCATGTGGCGGGTGGAGAGAGAAGGCACCG
GGCTCACGGAGGGCTGCCTGGTGTCCGTGGACGACTTGCAGAACCACGTG
GAGCACTTTGGGGAGGAGGAGCAGAAGGACCTGCGGGTGGACGTGGACA
CATTCTTGCAGTACTGGCCGCCGCAGAGCCAGCAGTTCAGCATGCAGTAC
ATCTCGCACATCTTCGGAGTGATTAACTGCAACGGTTTTACTCTCAGTGAT
CAGAGAGGCCTGCAGGCCGTGGGCGTAGGCATCTTCCCCAACCTGGGCCT
GGTGAACCATGACTGTTGGCCCAACTGTACTGTCATATTTAACAATGGCA
ATCATGAGGCAGTGAAATCCATGTTTCATACCCAGATGAGAATTGAGCTC
CGGGCCCTAGGCAAGATCTCAGAAGGAGAGGAGCTGACTGTGTCCTATAT
TGACTTCCTCAACGTTAGTGAAGAACGCAAGAGGCAGCTGAAGAAGCAGT
ACTACTTTGACTGCACATGTGAACACTGCCAGAAAAAACTGAAGGATGAC
CTCTTCCTGGGGGTGAAAGACAACCCCAAGCCCTCTCAGGAAGTGGTGAA
GGAGATGATACAATTCTCCAAGGATACATTGGAAAAGATAGACAAGGCTC
GTTCCGAGGGTTTGTATCATGAGGTTGTGAAATTATGCCGGGAGTGCCTG
GAGAAGCAGGAGCCAGTGTTTGCTGACACCAACATCTACATGCTGCGGAT
GCTGAGCATTGTTTCGGAGGTCCTTTCCTACCTCCAGGCCTTTGAGGAGGC
CTCGTTCTATGCCAGGAGGATGGTGGACGGCTATATGAAGCTCTACCACC
CCAACAATGCCCAACTGGGCATGGCCGTGATGCGGGCAGGGCTGACCAAC
TGGCATGCTGGTAACATTGAGGTGGGGCACGGGATGATCTGCAAAGCCTA
TGCCATTCTCCTGGTGACACACGGACCCTCCCACCCCATCACTAAGGACTT
AGAGGCCATGCGGGTGCAGACGGAGATGGAGCTACGCATGTTCCGCCAG
AACGAATTCATGTACTACAAGATGCGCGAGGCTGCCCTGAACAACCAGCC
CATGCAGGTCATGGCCGAGCCCAGCAATGAGCCATCCCCAGCTCTGTTCC
ACAAGAAGCAATGAGGACTGCCCAGTGGAGGAGGGGCGATGTGGCTGGG
GAGCTAGGGAGAGACTCTGGAGGTGGTGGGTCTCTCGGGAGACCCCTAAT
GAGGAAGTTGAGGTAATGCTTAACATTGTTGCTGTGAGAATTTACTGCCCT
ATGTTTCCCAGAGCCATTTTGGCTCAATTCAAGTCTATTCAATTCAAGTTA
ACTCTAGCCCAGCCCAGATCAACTCCTCCTACAAATATTATTGGATGATAG
GCCCTAGAACCCAATAAAGGAGCTCCAAATGTCGTTGGGTGGGGAAGCAA
AATGTAGAGAAACATTTAAAGCACACTGTAATAATAAATGCAATTATAAA
CTATATGGAGGAGGGTGCAGAGGAGGGAATGTGTCTGGTGTGTGATGTGT
GTGTGTGCAGTGGGGGTATCACAGAGAGTATGACATCTGAGTTGAGGGTA
GCAGGTGCCTGGAGTCTCAGGTGGCTGCTCACCCATCTGTGCAGGTGTCTC
TGGGGCTGCTGGTCTCACCTGTGGTCTGCAGTAGACACAATTGGCTGAGC
AGGATATGTGATACTGTGTGGTTGGTGTGGAGTTTTGAAGAAGGGGCTGT
GTTTGGGCCACGTAGGCTCTACTCAGAGACCTGAAACCACTTCAGAATGG
TGCATATGTCGAAAGAGCTGGCTGGGGGCCTTGCCCAAACCAACTGAGGT
CTTAAAGTCCAGGGAAAAAAAGTCTGGGTTCCAACTAGAATTCTAGAAAT
ATTTCTAGAACACACAGAGAGGGAATAAGTCCCTCTATCACCCTTATTAC
CAAGCCTTGTGGTTCCCTGTGATTTTAGATAATGTCTGATATTTTTCTGG
CTATTTGCCTAGTAGGATTTAAAAAATATTTTCAAAGTGAAGCTGAGAGA
GAATCTTGGAAACACACATACCTGTTGATCATGGGCCCTGCAGAATTGGC
CCTTGGGGGCTTTATTTGGTTACATGTGCCTGGGTGGTCTTTACCAGCTT
AGACTCTATCATGGGCCCCCATGAAGCTCCATTCTCAATACTGAATAATT
ATTACTTCCCTTGTTGAGTTTCTTTTTCTGTCATGCCCTGGGGGCTTCTG
CTCTTCTCACCAGAAAGAACATTTGAATCTGGATTCTTGTACACCTGGGT
TAGACCCTGTTCAGAGGTGTGGCCAATTTATCCCGATCTCCTGGAAGGCT
GTTGTGATTTCCATCTAAGAAATGAGGGTCTTGAGAATCAACCAGTCCCA
AGATTAGCCTGTTATCCTGTTATCTACTGAGACCCCAAATTTCTCACCAA
TGTTTTGGGAGATCCTGGAAAAGATCCCTTCAGTTTGGGGTGTCACCAAG
ACTTCTACACAACCCAGGACTACCATTGACCTCAGAGCTGTACCCCACAT
CTTGAAGTAAATTGATCCCACCAGGTCCCACGTTTGTTATCTCTGCCTAA
ATGTTAGCTTCTCCATCCTCACCACATGATGACCTGCTGTGTCCCTCTGA
GCACTACCCAGTGGCTGAAAACTCTGCAAATGGGCCACACTTTTGCAAAA
TACTTGTATCTGACACTTAGGTCTTGTTTGAAGAATTTCCTTTCTGGAAG
GTTTTACAAGAAGACTGATAGTCTTTCAAGCCCCCACATCACAGGCTTAG
GGACGGCACTAACTTTCTCCCAGGGATCTAACTGGCTAGTTCAAATTATC
ACTCTTTTACCTTCATATAAAATGTCTCCCCCAAACCTTTTTCCCTTCTT
TGTCATTGTTATCTGCTAAGCCCCTGGTCATTTCCCCATATTCGTAGTCT
TTTTTTCCATCCTATCTTTCTAATATTTGTTGTCTTTAACAAACTGTGTT
CTGTGTCTGTGCTCCTCCTTCCCTCTCAGACCACTGGAATGCAAGTCCTT
CTTCCCTTTGGAATGTACTCTGGATCCCTTCCCCTGCTTTGACCCCCAGA
CTTTGCTCCATCTATTATTGCTTCTCCATCCTGGATCCTTGACATTTGTC
ACCCCACTGGCCTTCTCAGGTGCAATCAGTAAAAATGCTGAGAACTCTTG
GATCTTAATCTTCATGACTGAGTTTTTTTTAGTTGTATAGTTATCATCTG
CCTTTCTTCACTTTGCATTTCTTCTTGAATCCATTGCAGATTGACTTCCA
CTCCCACTCCTTCACTAAAAGGGCTCTTACCAAGATCAAATCTAATGGGT
ACATTTTAGTTCCTATGTGATTTGGCCTTTCGATGTCAATCATCACTCCC
AGCCATTGATTTTGGTGACCCACTTCCCTGTGATGATCTTCTGATCTAGT
TTCTCAGGTTCCTTCGCTGGTCCTTTTTCTTTCCCTGCCCCTGACATATT
GACATTTCCTGGAGTTGGTTTTGTCCTTGATTCATTCTCATGTCATTCTG
CACACAGTCTCTGCATGAACTCAGGCAGACCCTTCATTTAATGACCACCT
TAGGGCTGATGATTCTCAAATCTGTATTCCCCGATCTTGCATTTGAGCTC
CAGCCCCACTCATCCTCTCGGATGTTCTGCAGGCCCAGCAAACTCATCAT
GTCCAAAGTGAAACTTTTTCTCTTTCCTGTCTCCTCTCCTCTGATCTGT
TCTTTCTTGGAACACCACCCAAGAACGTCACCTCCTCCATCAGATT
GTGAGCTCCTGGAGGGCAGGAGCTGTGTCCTTCTATTCATCTTCCTATCC
CCAGAACCTTGCACAGATCCTGGAATGTGGTAGGTGCTCAGTAAATGTGT
GTTGAATAAATGAATGAATGAATGAACAAATGAATGAATTTGCTTACTTC
AAGGCAAAAGAACCATGAAACTGTATTTTGAGTTTCTATGTTATAGCAGT
CAGCAAATCCTATTAAATACTTTGTGTTTCCAAGCAAA

46. The vector of any one of claims 42-45, wherein the nucleic acid construct comprises the sequence of SEQ ID NO:1.

47. The vector of any one of claims 42-45, wherein the nucleic acid construct comprises a nucleic acid sequence, wherein the nucleic acid sequence comprises an aMHC promoter sequence, a human SMYD1 gene sequence, a FLAG sequence, a WPRE sequence, and a polyA sequence, wherein the nucleic acid sequence is flanked by ITR sequences.

48. The vector of any one of claims 42-45, wherein the nucleic acid construct comprises a nucleic acid sequence, wherein the nucleic acid sequence comprises an αMHC promoter sequence, a human SMYD1 gene sequence, a GFP sequence, a WPRE sequence, and a poly A sequence, wherein the nucleic acid sequence is flanked by ITR sequences.


SEQUENCE LISTING (29 sequences)

SEQ ID NO: 1 - DNA, 18 bp, synthetic construct
  source 1..18: mol_type = other DNA
tgttaccaactgggacga

SEQ ID NO: 2 - DNA, 18 bp, synthetic construct
  source 1..18: mol_type = other DNA
ggggtgttgaaggtctca

SEQ ID NO: 3 - DNA, 23 bp, synthetic construct
  source 1..23: mol_type = other DNA
ctgatggatttcaagaacctgct

SEQ ID NO: 4 - DNA, 20 bp, synthetic construct
  source 1..20: mol_type = other DNA
ctctgggctccaatcctgtc

SEQ ID NO: 5 - DNA, 20 bp, synthetic construct
  source 1..20: mol_type = other DNA
gaacagctgggagaaggggg

SEQ ID NO: 6 - DNA, 22 bp, synthetic construct
  source 1..22: mol_type = other DNA
gcctctgaggctattctattgg

SEQ ID NO: 7 - DNA, 20 bp, synthetic construct
  source 1..20: mol_type = other DNA
gaacagctgggagaaggggg

SEQ ID NO: 8 - DNA, 22 bp, synthetic construct
  source 1..22: mol_type = other DNA
gcctctgaggctattctattgg

SEQ ID NO: 9 - DNA, 22 bp, synthetic construct
  source 1..22: mol_type = other DNA
ccttctaccagctgagtcattt

SEQ ID NO: 10 - DNA, 20 bp, synthetic construct
  source 1..20: mol_type = other DNA
cagatggagcccacgaccca

SEQ ID NO: 11 - DNA, 27 bp, synthetic construct
  source 1..27: mol_type = other DNA
cttcgagctgtacttttgtggacggaa

SEQ ID NO: 12 - DNA, 29 bp, synthetic construct
  source 1..29: mol_type = other DNA
ctctgagcttccttcagtaaactatcaaa

SEQ ID NO: 13 - DNA, 20 bp, synthetic construct
  source 1..20: mol_type = other DNA
tgagatcgccacctacagga

SEQ ID NO: 14 - DNA, 21 bp, synthetic construct
  source 1..21: mol_type = other DNA
tgatcacctgtccatctctgg

SEQ ID NO: 15 - DNA, 20 bp, synthetic construct
  source 1..20: mol_type = other DNA
ggtcagacctgtgtgttccc

SEQ ID NO: 16 - DNA, 20 bp, synthetic construct
  source 1..20: mol_type = other DNA
ggtccatgtaggctacgctg

SEQ ID NO: 17 - DNA, 29 bp, synthetic construct
  source 1..29: mol_type = other DNA
gtgaaatccatgtttcacacgcagatgag

SEQ ID NO: 18 - DNA, 22 bp, synthetic construct
  source 1..22: mol_type = other DNA
caggaagaggtcgtccttcagc

SEQ ID NO: 19 - DNA, 30 bp, synthetic construct
  source 1..30: mol_type = other DNA
ctgcactgtcatattcaacaatggcaagat

SEQ ID NO: 20 - DNA, 22 bp, synthetic construct
  source 1..22: mol_type = other DNA
caggaagaggtcgtccttcagc

SEQ ID NO: 21 - DNA, 20 bp, synthetic construct
  source 1..20: mol_type = other DNA
gcccattactgcgaccgcac

SEQ ID NO: 22 - DNA, 22 bp, synthetic construct
  source 1..22: mol_type = other DNA
ctccccaaagtgctccacgtgg

SEQ ID NO: 23 - DNA, 24 bp, synthetic construct
  source 1..24: mol_type = other DNA
ctcgctccgagggtttgtatcacg

SEQ ID NO: 24 - DNA, 28 bp, synthetic construct
  source 1..28: mol_type = other DNA
cttatcgtcgtcatccttgtaatccagg

SEQ ID NO: 25 - DNA, 20 bp, synthetic construct
  source 1..20: mol_type = other DNA
ggagggccaaacactacaaa

SEQ ID NO: 26 - DNA, 20 bp, synthetic construct
  source 1..20: mol_type = other DNA
tgagggagtacacggggtag

SEQ ID NO: 27 - DNA, 20 bp, synthetic construct
  source 1..20: mol_type = other DNA
tgatgaaagttggcaagctg

SEQ ID NO: 28 - DNA, 19 bp, synthetic construct
  source 1..19: mol_type = other DNA
gtccttctgggggtccttg

SEQ ID NO: 29 - DNA, 20 bp, synthetic construct
  source 1..20: mol_type = other DNA
cgaaccggtaaaccagagca